(19) United States
(12) Patent Application Publication
(10) Pub. No.: US 2003/0087411 A1
(43) Pub. Date: May 8, 2003

(54) Death associated kinase containing ankyr in repeats (DAKAR) and methods of use

(76) Inventors: Timothy A. Bird, Bainbridge Island, WA (US); Pamela M. Holland, Seattle, WA (US); Jacques J. Peschon, Seattle, WA (US); George D. Virca, Bellevue, WA (US)

Correspondence Address: IMMUNEX CORPORATION, LAW DEPARTMENT, 51 UNIVERSITY STREET, SEATTLE, WA, 98101

(21) Appl. No.: 10/164,080

(22) Filed: Jun. 4, 2002

Related U.S. Application Data
(60) Provisional application No. 60/295,959, filed on Jun. 4, 2001.
(60) Provisional application No. 60/334,362, filed on Nov. 29, 2001.

(51) Int. Cl. C12N 9/12; C07H 21/04; C12P 21/02; C12N 5/06

(52) U.S. Cl. 435/194; 435/69.1; 435/320.1; 435/325; 536/23.2

(57) ABSTRACT

This invention relates to DAKAR, a new member of the serine/threonine kinase family, methods of making such polypeptides, and to methods of using them to treat conditions associated with apoptosis and epithelial proliferation and differentiation, as well as methods to identify compounds that alter DAKAR-associated cellular activities.

### PRIORITY OF THE APPLICATION

This application claims the benefit of priority to U.S. Provisional Application No. 60/295,959, filed Jun. 4, 2001, and U.S. Provisional Application No. 60/334,362, filed Nov. 29, 2001.

### BACKGROUND

The present invention relates to nucleic acid and polypeptide sequences for DAKAR (Death Associated Kinase Containing Ankyrin Repeats), a new member of the serine/threonine protein kinase family, as well as agonists and antagonist of DAKAR and methods of use thereof. The intracellular expression of DAKAR is associated with apoptosis and keratinocyte differentiation. Sequences of the present invention may be used to design and create biological moieties that modulate the biological activity of DAKAR and/or DAKAR-related signaling pathways and thereby alleviate symptoms of various diseases and disorders.

DAKAR is a member of the serine/threonine protein kinase family and a mediator of apoptosis and putative modulator of cellular differentiation, proliferation, cell cycle and/or senescence.

Apoptosis is a form of programmed cell death, which is a developmental and homeostatic regulatory mechanism for removing superfluous, infected, transformed or damaged cells by activation of an intrinsic suicide program. Cells undergoing apoptosis usually exhibit a characteristic morphology, including blebbing of the plasma membrane, degradation of chromatin, initially into large fragments of 50-300 kilobases and subsequently into smaller fragments that are monomers and multimers of 200 bases.

In some instances, apoptosis may begin with a ligand/receptor induced signal that activates other proteins, such as kinases, along a signal transduction pathway that ultimately activates a cell death program. One apoptotic pathway is mediated by a cascade of cysteine proteases and caspases, which when activated, induce a series of degradative proteolytic events culminating in cell death. Ligand/receptor pairs that induce apoptosis are, for example, TNF/TNF-RI, TNF/TNF-R2, CD95 ligand/CD95, TRAIL/TRAIL-R1, and TRAIL/TRAIL-R2. In addition, apoptosis may be triggered by hormonal stimuli, such as glucocorticoid hormones for immature thymocytes, as well as withdrawal of certain growth factors. (Watanabe-Fukunaga et al., Nature, 356:314-317, 1992). Furthermore, oncogenes such as myc, re1, and E1A, and tumor suppressors such as p53, and select chemotherapy drugs and some forms of radiation have been shown to have apoptosis-inducing activity.

Members of the TNF superfamily ligands, such as TNF-α, TNF-β, CD30 ligand, CD27 ligand, CD40 ligand, OX-40 ligand, 4-1BB ligand, and Fas/Apo-1/CD95 ligand have been shown to be involved in apoptotic cell death. For example, TNF-α and TNF-β have been reported to induce apoptotic death in susceptible tumor cells (Schmid et al., PNAS, 83:1881 (1986) and Fas/Apo-1/CD95 ligand may be involved in the elimination of activated lymphocytes when their function is no longer needed (Nagata, et al., Science 267:1449-1456, 1995). Members of the TNF superfamily are typically type II transmembrane proteins and characterized by similar, cysteine-rich extra cellular domains and a homologous cytoplasmic sequence referred to as a “death domain.” Death domains typically enable death receptors to engage the cell's apoptotic pathway, but may also mediate functions that are distinct from or even counteract apoptosis. (Ashkenazi, A., et al., Science, 281:13, 1998). CD95 ligand binding to CD95 initiates association of “Fas-associated with death domain” protein (FADD or MORT-1) to the death domain of CD95 cytoplasmic tail via a homotypic death domain-to-death domain interaction. Additionally, CD95 also utilizes FADD to link cytoplasmic receptor sequences to caspase-8. Both FADD and caspase-8 interact through conserved death effector domains (DED) located in the pro-domain of caspase-8 and N-terminal region of FADD. Following ligand-induced oligimerization, the CD95 receptor recruits caspase-8 to the receptor signaling complex through FADD, which leads to the processing and release into the cytosol of caspase-8 and thereby inducing a cascade of caspases and the subsequent proteolytic degradation of the cell.

More recently, studies have shown that ligation of the extracellular domain of the cell surface receptor Fas/APO-1/CD95 elicits a characteristic apoptotic response in susceptible cells and that a serine/threonine kinase termed Receptor Interacting Protein (RIP) associates with the intracellular domain of Fas. RIP contains an N-terminal region with homology to protein kinases and a C-terminal region containing a cytoplasmic motif (death domain) present in the Fas and TNFR1 intracellular domains. Studies have shown that transient overexpression of RIP causes cells to undergo apoptosis. (Stanger, B. Z., et al., Cell, 81:513, 1995). In addition, RIP has been demonstrated to interact with the death receptor tumor necrosis receptor 1 (TNFR1). In vitro, RIP stimulates apoptosis, as well as SAPK/JNK and NF-κB activation. (Kelliher, M. A., et al., Immunity, 8:297, 1998). Furthermore, RICK (RIP-like interacting CLARP kinase) has been identified as a serine/threonine kinase having a C-terminal region containing a caspase-recruitment domain. RICK interacts with CLARP, a caspase-like molecule known to bind to FADD and caspase-8. Expression of RICK promoted activation of caspase-8 and potentiated apoptosis induced by Fas ligand, FADD, CLARP and caspase-8 (Inohara, N., et al., J. Bio. Chem., 273:20, 122296-300, 1998). Other protein kinases have been shown to be involved in apoptosis, such as DAP-Kinase Related Protein (DRP-1), which is a homologue of DAP-kinase. DRP-1 is a calmodulin-dependent kinase and is implicated as a cell death-promoting protein (WO 99/66030).

In addition to playing an integral role in apoptosis, protein kinases are thought to be involved in a number of complex signaling pathways affecting such diverse cellular processes as proliferation, differentiation and tumorigenesis. For example, RIPs have been shown to induce apoptosis and activate NF-κB, which has been implicated in mediating differentiation of stratified epithelium. In normal epidermis, NF-κB proteins were found to exist in the cytoplasm of basal cells and then localize in the nuclei of suprabasal cells, suggesting a role for NF-κB in the switch from proliferation to growth arrest and differentiation. Functional blockade of NF-κB by expressing dominant-negative NF-κB inhibitory proteins in transgenic murine and human epidermis produced hyperplastic epithelium in vivo and overexpresion of active p50 and p65 NF-κB subunits in transgenic epithelium produced hypoplasia and growth inhibition. Therefore, NF-κB undergoes nuclear translocation in a pattern that coincides spatially with the switch from the proliferative basal cell phenotype to the nonproliferative, terminal differentiation of the suprbasal layers, which suggests NF-κB may be necessary for the growth inhibition characteristic of upward migration and differentiation in stratified epithelium.

Compounds of the present invention have been shown to activate NF-κB and given that NF-κB is thought to have pleiotropic effects NF-κB in other tissues, NF-κB could alter epithelial growth via either a direct impact on cell cycle regulators or by a more indirect process. Such indirect effects could include the induction of growth inhibitory cytokines. Therefore agonists and/or antagonists of DAKAR may play a role in epithelial growth, differentiation, proliferation, cell cycle, and senescence, which has implications in a number of epithelial disorders. In addition, apoptosis is thought to play a role in the pathogenesis of a number of physiological and psychiatric disorders, such as stroke, ischemic injury, Alzheimer's disease, Parkinson's disease, Huntington's Chorea, multiple sclerosis, diabetic peripheral neuropathy, Amyotrophic Lateral Sclerosis (ALS), hereditary retinal degenerations, glaucoma, cachexia, and spinal muscular atrophy. Abnormalities in the differentiation process disrupt epithelial homeostasis and are characteristic of cutaneous neoplasms as well as a wide array of inflammatory skin diseases.

The present invention provides DAKAR compositions and methods of use for the diagnosis and treatment of DAKAR-associated disorders, as well as methods of identifying agonists and/or antagonists of DAKAR for the treatment of various diseases and disorders.

### SUMMARY

The present invention provides isolated mammalian Death Associated Kinase containing Ankyrin Repeats (“DAKAR”) polypeptides, as well as isolated polynucleotides encoding said polypeptides. Particular embodiments of the invention are directed to an isolated DAKAR nucleic acid molecule comprising the DNA sequence of SEQ ID NO:1 and an isolated DAKAR nucleic acid molecule encoding the amino acid sequence of SEQ ID NO:2, as well as nucleic acid molecules complementary to these sequences.

Other aspects of the invention relate to isolated nucleic acids encoding polypeptides of the invention, and isolated nucleic acids, preferably having a length of at least 15 nucleotides, that hybridize under conditions of moderate stringency to the nucleic acids encoding polypeptides of the invention. In preferred embodiments of the invention, such nucleic acids encode a polypeptide having DAKAR polypeptide activity, or comprise a nucleotide sequence that shares nucleotide sequence identity with the nucleotide sequences of the nucleic acids of the invention, wherein the percent nucleotide sequence identity is selected from the group consisting of: at least 70%, at least 75%, at least 80%, at least 85%, at least 90%, at least 95%, at least 97.5%, at least 99%, and at least 99.5%.

Murine DAKAR polynucleotide and corresponding polypeptide sequences as well as methods of use have been described in U.S. patent application Ser. No. 09/509,802, filed Aug. 4, 1999 based on International Patent Application No. PCT/US99/17576, U.S. Provisional Patent Application No. 60/119,353, filed Feb. 9, 1999, U.S. Provisional Patent Application No. 60/099,973, filed Sep. 11, 1998 and U.S. Provisional Patent Application No. 60/095,269, filed Aug. 4, 1998. Each of the aforementioned patent applications is incorporated by reference in their entireties.

In general, DAKAR polypeptides, polynucleotides, fragments, variants, muteins, fusion proteins, antagonists, agonists, antibodies, and binding partners etc. of the invention may be used to modulate apoptosis, differentiation, proliferation, cell cycle and/or senescence, immune regulation, cell migration, cell-to-cell interaction, and inflammatory responses in cells by either inhibiting or enhancing those pathways directly and/or indirectly through one or more DAKAR-associated events. In addition, these polypeptides can be used to identify proteins associated with DAKAR kinases.

In other aspects, the invention provides assays utilizing DAKAR polypeptides, polynucleotides, fragments, variants, muteins, fusion proteins, antagonists, agonists, antibodies, and binding partners etc. of the invention to screen for potential agonists and antagonists of DAKAR activity. Further, methods of using these DAKAR polypeptides, polynucleotides, fragments, variants, muteins, fusion proteins, antagonists, agonists, antibodies, and binding partners etc. in the design of inhibitors thereof are also an aspect of the invention.

Further provided by the invention are expression vectors and recombinant host cells comprising at least one nucleic acid of the invention, and preferred recombinant host cells wherein said nucleic acid is integrated into the host cell genome.

Also provided is a process for producing a polypeptide encoded by the nucleic acids of the invention, comprising culturing a recombinant host cell under conditions promoting expression of said polypeptide, wherein the recombinant host cell comprises at least one nucleic acid of the invention. A preferred process provided by the invention further comprises purifying said polypeptide. In another aspect of the invention, the polypeptide produced by said process is provided.

In another aspect, the present invention relates to a homozygous DAKAR deficient mouse strain, as well as any cells, cell lines, tissues and any biological moieties derived therefrom. Disruption of the DAKAR gene prevents the synthesis of functional DAKAR in the cells of the knockouts. The mice are characterized by a specific defect in keratinocyte growth, maturation, migration, differentiation, proliferation and the like that affects all keratinized squamous epithelia and results in perinatal lethality. DAKAR mutants have thickened non-wrinkled skin, poorly defined oral and auricular orifices, and the tail is fused to the skin in the area of the rectum and urethra opening resulting in no visible rectal or urethra orifice (anal and urethra atresia). Additionally, there was a high degree of atresia surrounding the mouth and ears. The limbs appeared rigid and somewhat stunted and the toes appear to be fused. Microscopically, DAKAR^{-/-} mutants consistently have markedly thickened corneal epithelium of the surface epidermis compared to wild-type embryos, as well as the absence of the stratum corneum. The corneal epithelium between the digits is partially fused and there is marked oral, anal, aral and esophageal atresia, as well as atresia in the anterior portion of the stomach.

In one particular embodiment, the introduced gene disruption comprises a deletion in a majority of the DAKAR gene. In an exemplary embodiment, a homologous recombination vector was constructed from a 5.4 Kb HindIII-Xba1 and a 1.3 Kb Nhe1-EcoR1 fragment from genomic clones so as to delete exons 2-7 and part of exon 8 thereby eliminating amino acids 62-492 and replacement of the deleted region with a PGKNeo and TK resistance cassettes as selection markers for neomycin resistance.

The invention additionally provides a method of designing an inhibitor of the polypeptides of the invention, the method comprising the steps of determining the three-dimensional structure of any such polypeptide, analyzing the three-dimensional structure for the likely binding sites of substrates, synthesizing a molecule that incorporates a predicted reactive site, and determining the polypeptide-inhibiting activity of the molecule.

The invention also provides a method for increasing DAKAR-associated apoptosis, differentiation, proliferation, cell cycle and/or senescence, immune regulation, cell migration, cell-to-cell interaction, and inflammatory responses activities, comprising providing at least one compound selected from the group consisting of the polypeptides of the invention and agonists of said polypeptides; with a preferred embodiment of the method further comprising increasing said activities in a patient by administering at least one polypeptide of the invention.

Further provided by the invention is a method for decreasing DAKAR-associated apoptosis, differentiation, proliferation, cell cycle and/or senescence, immune regulation, cell migration, cell-to-cell interaction, and inflammatory responses activity, comprising providing at least one antagonist of the polypeptides, polynucleotides, fragments, variants, muteins, fusion proteins, antagonists, agonists, antibodies, and binding partners etc. of the invention; with a preferred embodiment of the method further comprising decreasing said activities in a patient by administering at least one antagonist of the polypeptides, polynucleotides, fragments, variants, muteins, fusion proteins, antagonists, agonists, antibodies, and binding partners etc. of the invention.

The invention additionally provides a method for treating various epithelial disorders and diseases comprising administering at least one compound selected from the group consisting of the DAKAR polypeptides, polynucleotides, fragments, variants, muteins, fusion proteins, antagonists, agonists, antibodies, and binding partners etc. of the present invention; with a preferred embodiment wherein the epithelial disorder condition pertains to keratinocytes

In another aspect of the present invention a screening method is provided to identify active compounds that modulate DAKAR-associated cell signaling pathways and subsequent downstream events, including cellular pathways involved in apoptosis, differentiation, proliferation, cell cycle and/or senescence. An “active compound” as used herein, is a compound that modulates apoptosis, differentiation, proliferation, cell cycle and/or senescence in cells by either inhibiting or enhancing those pathways directly and/or indirectly through one or more DAKAR-associated events. The method includes contacting putative antagonists and/or agonists of apoptosis, differentiation, proliferation, cell cycle and/or senescence with cells expressing DAKAR, wherein apoptosis, differentiation, proliferation, cell cycle and/or senescence is inhibited or enhanced. The method includes assessing the ability of the active compounds to regulate apoptosis, differentiation, proliferation, cell cycle and/or senescence in cells. Alternatively, a similar method is provided to screen for antagonists and/or agonists of apoptosis, differentiation, proliferation, cell cycle and/or senescence on cells derived from DAKAR deficient mice to assess the efficacy of active compounds on those cellular pathways.

Further aspects of the invention are isolated antibodies that bind to the polypeptides of the invention, preferably monoclonal antibodies, also preferably humanized antibodies or human antibodies, and preferably wherein the antibody inhibits the activity of said polypeptides.

### BRIEF DESCRIPTION OF THE DRAWINGS

FIG. 1 shows the homology of the DAKAR ankyrin repeats to that of the known consensus ankyrin repeat sequence.

FIGS. 2A-C demonstrate that the involvement of DAKAR in apoptotic activity is independent of the kinase domain.

FIG. 3 summarizes the results showing DAKAR potentiated the activity of pro-apoptotic stimuli.

FIG. 4 represents a western blot showing DAKAR is cleaved by Caspase 3 and Caspase 8 in vitro.

### DETAILED DESCRIPTION

Nucleic Acids Encoding DAKAR Polypeptides

A cDNA clone for murine DAKAR is provided in SEQ ID NO:1, which was isolated as described in Example 1. The sequences of amino acids encoded by the open reading frame of SEQ ID NO: 1 is shown in SEQ ID NO:2. Based on homology of known polypeptides, DAKAR polypeptides are members of a family of protein kinases involved in apoptosis. The family of apoptotic proteins are characterized by an N-terminal protein serine-threonine kinase domain and a C-terminal domain which is variable and may include a series of ankyrin repeats, a death domain, or a caspase-recruitment domain. In the case of DAKAR, the C-terminal contains a series of ankyrin repeats, such as those known in other proteins to mediate protein-protein interactions. The kinase domain of DAKAR shares homology with three known proteins (RICK, DAPK-1, and RIP) that have been shown to play a role in apoptosis. DAKAR fusion proteins, truncation mutants and catalytic mutant sequences are described in detail in the Examples section below.

The cDNA sequence of human DAKAR (SEQ ID NO:6; huDAKAR) was identified by PCR from human fetal liver cDNA (Clontech) using primers based on mouse DAKAR sequence. A GenBank database search using huDAKAR cDNA shows that DAKAR maps to human chromosome 21 (Nature, 405:311-319). The precise location of huDAKAR on chromosome 21 is 21q22.3, in clone contig KB657H6. This region of chromosome 21 shows synteny with mouse chromosome 16, where muDAKAR resides.

Human DAKAR nucleic acids are expressed in various tissues, including kidney, liver, lung, placenta, colon, prostate, small intestine, thymus, and tonsils, as measured by RT-PCR assays.

Comparison of the sequences indicates that human and mouse DAKAR are 85% identical at the nucleotide level and 90% identical at the amino acid level.

Database analysis reveals that human DAKAR is made up of 8 exons. The boundaries for the exons are (relative to the polypeptide position of the encoded DAKAR sequence) at amino acid positions 59, 157, 207, 215, 265, 310, and 375 depicted in SEQ ID NO:7, corresponding to a nuceotide sequence spanning a region of roughly 26,000 nucleotides. The first 6 exons encode the catalytic domain, exon 7 spans the unique region, and exon 8 encompasses part of the unique region and all of the ankyrin repeats.

Further analysis reveled naturally occurring variants of DAKAR, including one having a valine for methionine substitution at amino acid 666 of SEQ ID NO:7, a variant comprising amino acids 26 to 784 of SEQ ID NO:7, a variant comprising amino acids 1 to 750 of SEQ ID NO:7, and another variant comprising amino aicds 26 to 750 of SEQ ID NO:7.

Encompassed within the invention are nucleic acids encoding DAKAR polypeptides. These nucleic acids can be identified in several ways, including isolation of genomic or cDNA molecules from a suitable source. Nucleotide sequences corresponding to the amino acid sequences described herein, to be used as probes or primers for the isolation of nucleic acids or as query sequences for database searches, can be obtained by “back-translation” from the amino acid sequences, or by identification of regions of amino acid identity with polypeptides for which the coding DNA sequence has been identified. The well-known polymerase chain reaction (PCR) procedure can be employed to isolate and amplify a DNA sequence encoding a DAKAR polypeptide or a desired combination of DAKAR polypeptide fragments. Oligonucleotides that define the desired termini of the combination of DNA fragments are employed as 5′ and 3′ primers. The oligonucleotides can additionally contain recognition sites for restriction endonucleases, to facilitate insertion of the amplified combination of DNA fragments into an expression vector. PCR techniques are described in Saiki et al., Science 239:487 (1988); Recombinant DNA Methodology, Wu et al., eds., Academic Press, Inc., San Diego (1989), pp. 189-196; and PCR Protocols: A Guide to Methods and Applications, Innis et. al., eds., Academic Press, Inc. (1990).

Nucleic acid molecules of the invention include DNA and RNA in both single-stranded and double-stranded form, as well as the corresponding complementary sequences. DNA includes, for example, cDNA, genomic DNA, chemically synthesized DNA, DNA amplified by PCR, and combinations thereof. The nucleic acid molecules of the invention include full-length genes or cDNA molecules as well as a combination of fragments thereof. The nucleic acids of the invention are preferentially derived from human sources, but the invention includes those derived from non-human species, as well.

An “isolated nucleic acid” is a nucleic acid that has been separated from adjacent genetic sequences present in the genome of the organism from which the nucleic acid was isolated, in the case of nucleic acids isolated from naturally-occurring sources. In the case of nucleic acids synthesized enzymatically from a template or chemically, such as PCR products, cDNA molecules, or oligonucleotides for example, it is understood that the nucleic acids resulting from such processes are isolated nucleic acids. An isolated nucleic acid molecule refers to a nucleic acid molecule in the form of a separate fragment or as a component of a larger nucleic acid construct. In one preferred embodiment, the nucleic acids are substantially free from contaminating endogenous material. The nucleic acid molecule has preferably been derived from DNA or RNA isolated at least once in substantially pure form and in a quantity or concentration enabling identification, manipulation, and recovery of its component nucleotide sequences by standard biochemical methods (such as those outlined in Sambrook et al., Molecular Cloning: A Laboratory Manual, 2nd ed., Cold Spring Harbor Laboratory, Cold Spring Harbor, N.Y. (1989)). Such sequences are preferably provided and/or constructed in the form of an open reading frame uninterrupted by internal non-translated sequences, or introns, that are typically present in eukaryotic genes. Sequences of non-translated DNA can be present 5′ or 3′ from an open reading frame, where the same do not interfere with manipulation or expression of the coding region.

The present invention also includes nucleic acids that hybridize under moderately stringent conditions, and more preferably highly stringent conditions, to nucleic acids encoding DAKAR polypeptides described herein. The basic parameters affecting the choice of hybridization conditions and guidance for devising suitable conditions are set forth by Sambrook, Fritsch, and Maniatis (1989, Molecular Cloning: A Laboratory Manual, Cold Spring Harbor Laboratory Press, Cold Spring Harbor, N.Y., chapters 9 and 11; and Current Protocols in Molecular Biology, 1995, Ausubel et al., eds., John Wiley & Sons, Inc., sections 2.10 and 6.3-6.4), and can be readily determined by those having ordinary skill in the art based on, for example, the length and/or base composition of the DNA. One way of achieving moderately stringent conditions involves the use of a prewashing solution containing 5× SSC, 0.5% SDS, 1.0 mM EDTA (pH 8.0), hybridization buffer of about 50% formamide, 6× SSC, and a hybridization temperature of about 55 degrees C. (or other similar hybridization solutions, such as one containing about 50% formamide, with a hybridization temperature of about 42 degrees C.), and washing conditions of about 60 degrees C., in 0.5× SSC, 0.1% SDS. Generally, highly stringent conditions are defined as hybridization conditions as above, but with washing at approximately 68 degrees C., 0.2× SSC, 0.1% SDS. SSPE (1× SSPE is 0.15M NaCl, 10 mM NaH.sub.2 PO.sub.4, and 1.25 mM EDTA, pH 7.4) can be substituted for SSC (1× SSC is 0.15M NaCl and 15 mM sodium citrate) in the hybridization and wash buffers; washes are performed for 15 minutes after hybridization is complete. It should be understood that the wash temperature and wash salt concentration can be adjusted as necessary to achieve a desired degree of stringency by applying the basic principles that govern hybridization reactions and duplex stability, as known to those skilled in the art and described further below (see, e.g., Sambrook et al., 1989). When hybridizing a nucleic acid to a target nucleic acid of unknown sequence, the hybrid length is assumed to be that of the hybridizing nucleic acid. When nucleic acids of known sequence are hybridized, the hybrid length can be determined by aligning the sequences of the nucleic acids and identifying the region and/or regions of optimal sequence complementarity. The hybridization temperature for hybrids anticipated to be less than 50 base pairs in length should be 5 to 10.degrees C. less than the melting temperature (Tm) of the hybrid, where Tm is determined according to the following equations. For hybrids less than 18 base pairs in length, Tm (degrees C.)=2(# of A+T bases)+4(# of #G+C bases). For hybrids above 18 base pairs in length, Tm (degrees C.)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1× SSC=0. 165M).

Each hybridizing nucleic acid has a length that can be at least 15 nucleotides (or more preferably at least 18 nucleotides, or at least 20 nucleotides, or at least 25 nucleotides, or at least 30 nucleotides, or at least 40 nucleotides, or most preferably at least 50 nucleotides), or at least 25% (more preferably at least 50%, or at least 60%, or at least 70%, and most preferably at least 80%) of the length of the nucleic acid of the present invention to which it hybridizes, and has at least 60% sequence identity (more preferably at least 70%, at least 75%, at least 80%, at least 85%, at least 90%, at least 95%, at least 97.5%, or at least 99%, and most preferably at least 99.5%) with the nucleic acid of the present invention to which it hybridizes, where sequence identity is determined by comparing the sequences of the hybridizing nucleic acids when aligned so as to maximize overlap and identity while minimizing sequence gaps as described in more detail above.

The present invention also provides genes corresponding to the nucleic acid sequences disclosed herein. “Corresponding genes” or “corresponding genomic nucleic acids” are the regions of the genome that are transcribed to produce the mRNAs from which cDNA nucleic acid sequences are derived and can include contiguous regions of the genome necessary for the regulated expression of such genes. Corresponding genes can therefore include but are not limited to coding sequences, 5′ and 3′ untranslated regions, alternatively spliced exons, introns, promoters, enhancers, and silencer or suppressor elements. Corresponding genomic nucleic acids can include 10000 basepairs (more preferably, 5000 basepairs, still more preferably, 2500 basepairs, and most preferably, 1000 basepairs) of genomic nucleic acid sequence upstream of the first nucleotide of the genomic sequence corresponding to the initiation codon of the DAKAR coding sequence, and 10000 basepairs (more preferably, 5000 basepairs, still more preferably, 2500 basepairs, and most preferably, 1000 basepairs) of genomic nucleic acid sequence downstream of the last nucleotide of the genomic sequence corresponding to the termination codon of the DAKAR coding sequence.

The corresponding genes or genomic nucleic acids can be isolated in accordance with known methods using the sequence information disclosed herein. Such methods include the preparation of probes or primers from the disclosed sequence information for identification and/or amplification of genes in appropriate genomic libraries or other sources of genomic materials. An “isolated gene” or “an isolated genomic nucleic acid” is a genomic nucleic acid that has been separated from the adjacent genomic sequences present in the genome of the organism from which the genomic nucleic acid was isolated.

Similarities of DAKAR Structure to Other Serine/Threonine Kinase Family Members

We have identified DAKAR, a new serine/threonine kinase polypeptide having structural features characteristic of this polypeptide family; the amino acid sequence of a murine DAKAR polypeptide is provided in SEQ ID NO:2 and the human DAKAR amino acid sequence provided in SEQ ID NO:7. Cloning and sequencing of DAKAR indicated that its catalytic domain has similarity to a growing family of apoptosis-inducing kinases known as RIPs (receptor interacting protein). At least three members (RIP, RIP2/CARDIAK/RICK, and RIP3) have been identified to date. RIPs are components of signaling complexes that are recruited to the Fas or TNF receptor. DAKAR contains nine ankyrin repeats in its C-terminal regulatory domain in contrast to RIP, RIP2 and RIP3, which contain a C-terminal death domain (DD), a caspase activation and recruitment domain (CARD), or a unique C-terminal domain, respectively. RIP, RIP2 and RIP3 can all potently induce apoptosis and activate NFκB when overexpressed in cells, and in all cases the catalytic domain is not required.

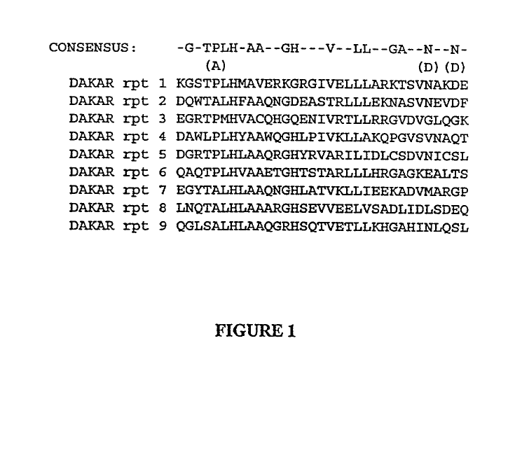

HuDAKAR is made up of 8 exons spanning a region of roughly 26,000 nucleotides. The first 6 exons encode the catalytic domain, exon 7 spans the unique region, and exon 8 encompasses part of the unique region and all of the ankyrin repeats. The ankyrin repeats of DAKAR are aligned sequentially under a consensus ankyrin repeat in FIG. 1.

Human DAKAR also has a number of putative PKC phosphorylation sites and therefore may interact with the catalytic domain of PKC isoforms, suggesting DAKAR may associate with multiple PKCs.

Human DAKAR also has a caspase cleavage site at amino acids 433 to 437 of SEQ ID NO:7.

The typical structural elements common to members of the DAKAR polypeptide family include one or more catalytic or kinase domains (from amino acids 22 to 302 of SEQ ID NO:7), a putative nuclear localization site (at amino acids 469 to 482 of SEQ ID NO:7), ankyrin repeat domains, a number of PKC phosphorylation sites and a unique carboxy terminus. The DAKAR kinase domain is found at the N-terminus of DAKAR family polypeptides, and is followed, in N-to-C order, by an ankyrin repeat domain. The DAKAR polypeptide has a characteristic catalytic loop domain (DLKPAN) within the kinase domain at amino acids 142 to 148 of SEQ ID NO:7.

In addition, there are some key amino acids within the kinase domain, such as the conserved lysine in the catalytic loop of the kinase domain. Also, the tertiary/three-dimensional structure of the ankyrin repeat domains may be implicated in activity of the subcellular localization of DAKAR family members. The skilled artisan will recognize that the boundaries of the regions of DAKAR polypeptides described above are approximate and that the precise boundaries of such domains, as for example the boundaries of the kinase domain or nuclear localization site (which can be predicted by using computer programs available for that purpose), can also differ from member to member within naturally occurring variants of the DAKAR polypeptide family.

To further establish the classification of DAKAR as a member of the serine/threonine kinase polypeptide family, the DAKAR sequence was submitted to GeneFold (Tripos, Inc., St. Louis, Mo.; Berman et al., 2000, Nucleic Acids Res 28:235-242), a protein threading program that overlays a query protein sequence onto structural representatives of the Protein Data Bank (PDB) (Jaroszewski et al., 1998, Prot Sci 7:1431-1440). Serine/threonine kinase family members are characterized by a three-dimensional structure that can be predicted from their primary amino acid sequences by using protein-threading algorithms such as GeneFold. To use GeneFold to classify new members of a protein family, the new protein sequence is entered into the program, then is assigned a probability score that reflects how well it folds onto previously known protein structures (“template” structures) that are present in the GeneFold database. For scoring, GeneFold relies on primary amino acid sequence similarity, burial patterns of residues, local interactions and secondary structure comparisons. The GeneFold program folds (or threads) the amino acid sequence onto all of the template structures in a preexisting database of protein folds, which includes the solved structures for several serine/threonine kinase polypeptides. For each comparison, three different scores are calculated, based on (i) sequence only; (ii) sequence plus local conformation preferences plus burial terms; and (iii) sequence plus local conformation preferences plus burial terms plus secondary structure. In each instance, the program determines the optimal alignment, calculates the probability (P-value) that this degree of alignment occurred by chance, and reports the inverse of the P-value as the score with 999.9 (9.999×10²) being the highest possible score. These scores therefore reflect the degree to which the new protein matches the various reference structures and are useful for assigning a new protein to membership in a known family of proteins.

DAKAR Polypeptides

A human DAKAR polypeptide is a polypeptide that shares a sufficient degree of amino acid identity or similarity to the human DAKAR polypeptide of SEQ ID NO:7 to (A) be identified by those of skill in the art as a polypeptide likely to share particular structural domains and/or (B) have biological activities in common with the DAKAR polypeptidesand/or (C) bind to antibodies that also specifically bind to other DAKAR polypeptides. DAKAR polypeptides can be isolated from naturally occurring sources, or have the same structure as naturally occurring DAKAR polypeptides, or can be produced to have structures that differ from naturally occurring DAKAR polypeptides. Polypeptides derived from any DAKAR polypeptide by any type of alteration (for example, but not limited to, insertions, deletions, or substitutions of amino acids; changes in the state of glycosylation of the polypeptide; refolding or isomerization to change its three-dimensional structure or self-association state; and changes to its association with other polypeptides and/or molecules) are also DAKAR polypeptides. Therefore, the polypeptides provided by the invention include polypeptides characterized by amino acid sequences similar to those of the DAKAR polypeptides described herein, but into which modifications are naturally provided or deliberately engineered.

Human DAKAR polypeptides are expressed in various tissues, including kidney, liver, lung, placenta, colon, prostate, small intestine, thymus, and tonsils.

A polypeptide that shares biological activities in common with DAKAR polypeptides is a polypeptide having DAKAR polypeptide activity and includes agonists and antagonists thereof. Examples of biological activities exhibited by DAKAR polypeptides include, without limitation, kinase activity, apoptosis, cellular differentiation, proliferation, cell cycle and/or senescence, and in particular epithelial and keratinocyte apoptosis, cellular differentiation, proliferation, cell cycle and/or senescence. Other biological activities may include, for example, activation of NFκB; association with the IκB kinase family and associated subunits such as IKKα; IKKβ and IKKγ, and the like; regulation of keratinocyte differentiation genes such as filaggrin, involucrin and various keratin markers such as K5, K6, K14, and the like.

The present invention provides both full-length and mature forms of DAKAR polypeptides. Full-length polypeptides are those having the complete primary amino acid sequence of the polypeptide as initially translated. The amino acid sequences of full-length polypeptides can be obtained, for example, by translation of the complete open reading frame (“ORF”) of a cDNA molecule. Several full-length polypeptides can be encoded by a single genetic locus if multiple mRNA forms are produced from that locus by alternative splicing or by the use of multiple translation initiation sites. The “mature form” of a polypeptide refers to a polypeptide that has undergone post-translational processing steps such as cleavage of the signal sequence or proteolytic cleavage to remove a prodomain. Multiple mature forms of a particular full-length polypeptide may be produced, for example by cleavage of the signal sequence at multiple sites, or by differential regulation of proteases that cleave the polypeptide. The mature form(s) of such polypeptide can be obtained by expression, in a suitable mammalian cell or other host cell, of a nucleic acid molecule that encodes the full-length polypeptide. The sequence of the mature form of the polypeptide may also be determinable from the amino acid sequence of the full-length form, through identification of signal sequences or protease cleavage sites.

The DAKAR polypeptides of the invention also include those that result from post-transcriptional or post-translational processing events such as alternate mRNA processing which can yield a truncated but biologically active polypeptide, for example, a naturally occurring soluble form of the polypeptide. Also encompassed within the invention are variations attributable to proteolysis such as differences in the N- or C-termini upon expression in different types of host cells, due to proteolytic removal of one or more terminal amino acids from the polypeptide (generally from 1-5 terminal amino acids).

The invention further includes DAKAR polypeptides with or without associated native-pattern glycosylation. Polypeptides expressed in yeast or mammalian expression systems (e.g., COS-1 or CHO cells) can be similar to or significantly different from a native polypeptide in molecular weight and glycosylation pattern, depending upon the choice of expression system. Expression of polypeptides of the invention in bacterial expression systems, such as E. coli, provides non-glycosylated molecules. Further, a given preparation can include multiple differentially glycosylated species of the polypeptide. Glycosyl groups can be removed through conventional methods, in particular those utilizing glycopeptidase. In general, glycosylated polypeptides of the invention can be incubated with a molar excess of glycopeptidase (Boehringer Mannheim).

Species homologues of DAKAR polypeptides and of nucleic acids encoding them are also provided by the present invention. As used herein, a “species homologue” is a polypeptide or nucleic acid with a different species of origin from that of a given polypeptide or nucleic acid, but with significant sequence similarity to the given polypeptide or nucleic acid, as determined by those of skill in the art. Species homologues can be isolated and identified by making suitable probes or primers from polynucleotides encoding the amino acid sequences provided herein and screening a suitable nucleic acid source from the desired species. The invention also encompasses allelic variants of DAKAR polypeptides and nucleic acids encoding them; that is, naturally-occurring alternative forms of such polypeptides and nucleic acids in which differences in amino acid or nucleotide sequence are attributable to genetic polymorphism (allelic variation among individuals within a population).

Fragments of the DAKAR polypeptides of the present invention are encompassed by the present invention and can be in linear form or cyclized using known methods, for example, as described in Saragovi et al., Bio/Technology 10:773-778 (1992) and in McDowell et al., J. Amer. Chem. Soc. 114:9245-9253 (1992).

As described in detail below, fragments of the present invention may comprise any contiguous section of SEQ ID NOs:2 or 7. One embodiment of the present invention encompasses DAKAR fragments comprising the kinase domain and fragments thereof. For example, the kinase domain of DAKAR, which may comprise amino acids a to b of SEQ ID NOs:2 or 7, wherein a represents an integer from 1 to 150 and b represents an integer from 151 to 320. Another embodiment of the present invention encompasses DAKAR fragments comprising the ankyrin repeat region and fragments thereof. For example, the ankyrin repeat domain of DAKAR, which may comprise amino acids c to d of SEQ ID NOs:2 or 7, wherein c represents an integer from 400 to 600 and d represents an integer from 601 to 780. Other embodiments may comprise the nuclear localization site of DAKAR, which may comprise amino acids e to f of SEQ ID NOs:2 or 7 wherein e represents an integer from 450 to 480 and d represents an integer from 451 to 500

Polypeptides and polypeptide fragments of the present invention, and nucleic acids encoding them, include polypeptides and nucleic acids with amino acid or nucleotide sequence lengths that are at least 25% (more preferably at least 50%, or at least 60%, or at least 70%, and most preferably at least 80%) of the length of a DAKAR polypeptide and have at least 60% sequence identity (more preferably at least 70%, at least 75%, at least 80%, at least 85%, at least 90%, at least 95%, at least 97.5%, or at least 99%, and most preferably at least 99.5%) with that DAKAR polypeptide or encoding nucleic acid, where sequence identity is determined by comparing the amino acid sequences of the polypeptides when aligned so as to maximize overlap and identity while minimizing sequence gaps. Also included in the present invention are polypeptides and polypeptide fragments, and nucleic acids encoding them, that contain or encode a segment preferably comprising at least 8, or at least 10, or preferably at least 15, or more preferably at least 20, or still more preferably at least 30, or most preferably at least 40 contiguous amino acids. Such polypeptides and polypeptide fragments may also contain a segment that shares at least 70% sequence identity (more preferably at least 70%, at least 75%, at least 80%, at least 85%, at least 90%, at least 95%, at least 97.5%, or at least 99%, and most preferably at least 99.5%) with any such segment of any DAKAR polypeptide, where sequence identity is determined by comparing the amino acid sequences of the polypeptides when aligned so as to maximize overlap and identity while minimizing sequence gaps. The percent identity of two amino acid or two nucleic acid sequences can be determined by visual inspection and mathematical calculation, or more preferably, the comparison is done by comparing sequence information using a computer program. An exemplary, preferred computer program is the Genetics Computer Group (GCG; Madison, Wis.) Wisconsin package version 10.0 program, ‘GAP’ (Devereux et al., 1984, Nucl. Acids Res. 12:387). The preferred default parameters for the ‘GAP’ program includes: (1) The GCG implementation of a unary comparison matrix (containing a value of 1 for identities and 0 for non-identities) for nucleotides, and the weighted amino acid comparison matrix of Gribskov and Burgess, Nucl. Acids Res. 14:6745, 1986, as described by Schwartz and Dayhoff, eds., Atlas of Polypeptide Sequence and Structure, National Biomedical Research Foundation, pp. 353-358, 1979; or other comparable comparison matrices; (2) a penalty of 30 for each gap and an additional penalty of 1 for each symbol in each gap for amino acid sequences, or penalty of 50 for each gap and an additional penalty of 3 for each symbol in each gap for nucleotide sequences; (3) no penalty for end gaps; and (4) no maximum penalty for long gaps. Other programs used by those skilled in the art of sequence comparison can also be used, such as, for example, the BLASTN program version 2.0.9, available for use via the National Library of Medicine website www.ncbi.nlm.nih.gov/gorf/wblast2.cgi, or the UW-BLAST 2.0 algorithm. Standard default parameter settings for UW-BLAST 2.0 are described at the following Internet site: sapiens.wustl.edu/blast/blast/#Features. In addition, the BLAST algorithm uses the BLOSUM62 amino acid scoring matix, and optional parameters that can be used are as follows: (A) inclusion of a filter to mask segments of the query sequence that have low compositional complexity (as determined by the SEG program of Wootton and Federhen (Computers and Chemistry, 1993); also see Wootton and Federhen, 1996, Analysis of compositionally biased regions in sequence databases, Methods Enzymol. 266:554-71) or segments consisting of short-periodicity internal repeats (as determined by the XNU program of Claverie and States (Computers and Chemistry, 1993)), and (B) a statistical significance threshold for reporting matches against database sequences, or E-score (the expected probability of matches being found merely by chance, according to the stochastic model of Karlin and Altschul (1990); if the statistical significance ascribed to a match is greater than this E-score threshold, the match will not be reported.); preferred E-score threshold values are 0.5, or in order of increasing preference, 0.25, 0.1, 0.05, 0.01, 0.001, 0.0001, 1e-5, 1e-10, 1e-15, 1e-20, 1e-25, 1e-30, 1e-40, 1e-50, 1e-75, or 1e-100.

The present invention also provides for soluble forms of DAKAR polypeptides comprising certain fragments or domains of these polypeptides, and particularly those comprising the extracellular domain or one or more fragments of the extracellular domain. Soluble polypeptides are polypeptides that are capable of being secreted from the cells in which they are expressed. In such forms part or all of the intracellular and transmembrane domains of the polypeptide are deleted such that the polypeptide is fully secreted from the cell in which it is expressed. The intracellular and transmembrane domains of polypeptides of the invention can be identified in accordance with known techniques for determination of such domains from sequence information. Soluble DAKAR polypeptides also include those polypeptides which include part of the transmembrane region, provided that the soluble DAKAR polypeptide is capable of being secreted from a cell, and preferably retains DAKAR polypeptide activity. Soluble DAKAR polypeptides further include oligomers or fusion polypeptides comprising the extracellular portion of at least one DAKAR polypeptide, and fragments of any of these polypeptides that have DAKAR polypeptide activity. A secreted soluble polypeptide can be identified (and distinguished from its non-soluble membrane-bound counterparts) by separating intact cells which express the desired polypeptide from the culture medium, e.g., by centrifugation, and assaying the medium (supernatant) for the presence of the desired polypeptide. The presence of the desired polypeptide in the medium indicates that the polypeptide was secreted from the cells and thus is a soluble form of the polypeptide. The use of soluble forms of DAKAR polypeptides is advantageous for many applications. Purification of the polypeptides from recombinant host cells is facilitated, since the soluble polypeptides are secreted from the cells. Moreover, soluble polypeptides are generally more suitable than membrane-bound forms for parenteral administration and for many enzymatic procedures.

In another aspect of the invention, preferred polypeptides comprise one or more of various of DAKAR polypeptide domains, as well as any combinations thereof, such as the kinase domain, nuclear localization domain, phosphorylation sites and/or all or some of the ankyrin repeat domain. Accordingly, polypeptides of the present invention and nucleic acids encoding them include those comprising or encoding two or more copies of one or more domains, such as the one or more copies of the kinase domain, nuclear localization domain, phosphorylation sites and/or the ankyrin repeat domain, as well as any permutations therein. Furthermore, these domains can be presented in any order within such polypeptides.

Further modifications in the peptide or DNA sequences can be made by those skilled in the art using known techniques. Modifications of interest in the polypeptide sequences can include the alteration, substitution, replacement, insertion or deletion of a selected amino acid. For example, a variant of DAKAR is the substitution of a methionine for a valine at amino acid 666. In one embodiment, a catalytically inactive DAKAR mutant is presented which is described in detail in Example 7. A similar catalytically inactive mutant for huDAKAR may be made using similar techniques.

In further examples, one or more of the cysteine residues can be deleted or replaced with another amino acid to alter the conformation of the molecule, an alteration which may involve preventing formation of incorrect intramolecular disulfide bridges upon folding or renaturation. Techniques for such alteration, substitution, replacement, insertion or deletion are well known to those skilled in the art (see, e.g., U.S. Pat. No. 4,518,584). As another example, N-glycosylation sites in the polypeptide extracellular domain can be modified to preclude glycosylation, allowing expression of a reduced carbohydrate analog in mammalian and yeast expression systems. N-glycosylation sites in eukaryotic polypeptides are characterized by an amino acid triplet Asn-X-Y, wherein X is any amino acid except Pro and Y is Ser or Thr. Appropriate substitutions, additions, or deletions to the nucleotide sequence encoding these triplets will result in prevention of attachment of carbohydrate residues at the Asn side chain. Alteration of a single nucleotide, chosen so that Asn is replaced by a different amino acid, for example, is sufficient to inactivate an N-glycosylation site. Alternatively, the Ser or Thr can by replaced with another amino acid, such as Ala. Known procedures for inactivating N-glycosylation sites in polypeptides include those described in U.S. Pat. No. 5,071,972 and EP 276,846. Additional variants within the scope of the invention include polypeptides that can be modified to create derivatives thereof by forming covalent or aggregative conjugates with other chemical moieties, such as glycosyl groups, lipids, phosphate, acetyl groups and the like. Covalent derivatives can be prepared by linking the chemical moieties to functional groups on amino acid side chains or at the N-terminus or C-terminus of a polypeptide. Conjugates comprising diagnostic (detectable) or therapeutic agents attached thereto are contemplated herein. Preferably, such alteration, substitution, replacement, insertion or deletion retains the desired activity of the polypeptide or a substantial equivalent thereof. One example is a variant that binds with essentially the same binding affinity as does the native form. Binding affinity can be measured by conventional procedures, e.g., as described in U.S. Pat. No. 5,512,457 and as set forth herein.

Other derivatives include covalent or aggregative conjugates of the polypeptides with other polypeptides and/or polypeptides, such as by synthesis in recombinant culture as N-terminal or C-terminal fusions. Examples of fusion polypeptides are discussed below in connection with oligomers. Further, fusion polypeptides can comprise peptides added to facilitate purification and identification. Such peptides include, for example, poly-His or the antigenic identification peptides described in U.S. Pat. No. 5,011,912 and in Hopp et al., Bio/Technology 6:1204, 1988. One such peptide is the FLAG® peptide, which is highly antigenic and provides an epitope reversibly bound by a specific monoclonal antibody, enabling rapid assay and facile purification of expressed recombinant polypeptide. A murine hybridoma designated 4E11 produces a monoclonal antibody that binds the FLAG® peptide in the presence of certain divalent metal cations, as described in U.S. Pat. No. 5,011,912. The 4E11 hybridoma cell line has been deposited with the American Type Culture Collection under accession no. HB 9259. Monoclonal antibodies that bind the FLAG® peptide are available from Eastman Kodak Co., Scientific Imaging Systems Division, New Haven, Conn.

Encompassed by the invention are oligomers and/or fusion polypeptides that contain a DAKAR polypeptide, one or more fragments of DAKAR polypeptides, or any of the derivative or variant forms of DAKAR polypeptides as disclosed herein. In particular embodiments, the oligomers comprise soluble DAKAR polypeptides. Oligomers can be in the form of covalently linked or non-covalently-linked multimers, including dimers, trimers, or higher oligomers. In one aspect of the invention, the oligomers maintain the binding ability of the polypeptide components and provide therefor, bivalent, trivalent, etc., binding sites. In an alternative embodiment the invention is directed to oligomers comprising multiple DAKAR polypeptides joined via covalent or non-covalent interactions between peptide moieties fused to the polypeptides, such peptides having the property of promoting oligomerization. Leucine zippers and certain polypeptides derived from antibodies are among the peptides that can promote oligomerization of the polypeptides attached thereto, as described in more detail below.

In embodiments where variants of the DAKAR polypeptides are constructed to include a membrane-spanning domain, they will form a Type I membrane polypeptide. Membrane-spanning DAKAR polypeptides can be fused with extracellular domains of receptor polypeptides for which the ligand is known. Such fusion polypeptides can then be manipulated to control the intracellular signaling pathways triggered by the membrane-spanning DAKAR polypeptide. DAKAR polypeptides that span the cell membrane can also be fused with agonists or antagonists of cell-surface receptors, or cellular adhesion molecules to further modulate DAKAR intracellular effects. In another aspect of the present invention, interleukins can be situated between the preferred DAKAR polypeptide fragment and other fusion polypeptide domains.

Immunoglobulin-Based Oligomers. The polypeptides of the invention or fragments thereof can be fused to molecules such as immunoglobulins for many purposes, including increasing the valence of polypeptide binding sites. For example, fragments of a DAKAR polypeptide can be fused directly or through linker sequences to the Fc portion of an immunoglobulin. For a bivalent form of the polypeptide, such a fusion could be to the Fc portion of an IgG molecule. Other immunoglobulin isotypes can also be used to generate such fusions. For example, a polypeptide-IgM fusion would generate a decavalent form of the polypeptide of the invention. The term “Fc polypeptide” as used herein includes native and mutein forms of polypeptides made up of the Fc region of an antibody comprising any or all of the CH domains of the Fc region. Truncated forms of such polypeptides containing the hinge region that promotes dimerization are also included. Preferred Fc polypeptides comprise an Fc polypeptide derived from a human IgG1 antibody. As one alternative, an oligomer is prepared using polypeptides derived from immunoglobulins. Preparation of fusion polypeptides comprising certain heterologous polypeptides fused to various portions of antibody-derived polypeptides (including the Fc domain) has been described, e.g., by Ashkenazi et al. (PNAS USA 88:10535, 1991); Byrn et al. (Nature 344:677, 1990); and Hollenbaugh and Aruffo (“Construction of Immunoglobulin Fusion Polypeptides”, in Current Protocols in Immunology, Suppl. 4, pages 10.19.1-10.19.11, 1992). Methods for preparation and use of immunoglobulin-based oligomers are well known in the art. One embodiment of the present invention is directed to a dimer comprising two fusion polypeptides created by fusing a polypeptide of the invention to an Fc polypeptide derived from an antibody. A gene fusion encoding the polypeptide/Fc fusion polypeptide is inserted into an appropriate expression vector. Polypeptide/Fc fusion polypeptides are expressed in host cells transformed with the recombinant expression vector, and allowed to assemble much like antibody molecules, whereupon interchain disulfide bonds form between the Fc moieties to yield divalent molecules. One suitable Fc polypeptide, described in PCT application WO 93/10151, is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., (EMBO J. 13:3992-4001, 1994). The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors. The above-described fusion polypeptides comprising Fc moieties (and oligomers formed therefrom) offer the advantage of facile purification by affinity chromatography over Polypeptide A or Polypeptide G columns. In other embodiments, the polypeptides of the invention can be substituted for the variable portion of an antibody heavy or light chain. If fusion polypeptides are made with both heavy and light chains of an antibody, it is possible to form an oligomer with as many as four DAKAR extracellular regions.

Peptide-Linker Based Oligomers. Alternatively, the oligomer is a fusion polypeptide comprising multiple DAKAR polypeptides, with or without peptide linkers (spacer peptides). Among the suitable peptide linkers are those described in U.S. Pat. Nos. 4,751,180 and 4,935,233. A DNA sequence encoding a desired peptide linker can be inserted between, and in the same reading frame as, the DNA sequences of the invention, using any suitable conventional technique. For example, a chemically synthesized oligonucleotide encoding the linker can be ligated between the sequences. In particular embodiments, a fusion polypeptide comprises from two to four soluble DAKAR polypeptides, separated by peptide linkers. Suitable peptide linkers, their combination with other polypeptides, and their use are well known by those skilled in the art.

Leucine-Zippers. Another method for preparing the oligomers of the invention involves use of a leucine zipper. Leucine zipper domains are peptides that promote oligomerization of the polypeptides in which they are found. Leucine zippers were originally identified in several DNA-binding polypeptides (Landschulz et al., Science 240:1759, 1988), and have since been found in a variety of different polypeptides. Among the known leucine zippers are naturally occurring peptides and derivatives thereof that dimerize or trimerize. The zipper domain (also referred to herein as an oligomerizing, or oligomer-forming, domain) comprises a repetitive heptad repeat, often with four or five leucine residues interspersed with other amino acids. Use of leucine zippers and preparation of oligomers using leucine zippers are well known in the art. Specific nonlimiting examples of leucine zipper sequences include the sequence derived from surfactant protein D (SPD; SEQ ID NO:4) and GCN4 (SEQ ID NO:5).

Other fragments and derivatives of the sequences of polypeptides which would be expected to retain polypeptide activity in whole or in part and may thus be useful for screening or other immunological methodologies can also be made by those skilled in the art given the disclosures herein. Such modifications are believed to be encompassed by the present invention.

Biological Activities and Functions of DAKAR Polypeptides

Overexpression of the DAKAR-FLAG® fusion protein and the dominant negative mutant DAKAR-A51 (as described in Example 8) in 293 in EBNA cells activates pro-apoptotic pathways, such as the cleavage of caspase-3, cleavage of the intracellular caspase substrates fodrin and poly(ADP-ribose)polymerase (PARP), as well as other well-characterized apoptotic stimuli, such as TNFα, TRAIL or anti-Fas antibody. Although expressed at lower levels than wild-type DAKAR, mutated DAKAR also induced apoptotic changes similar to those described above, thereby demonstrating that DAKAR does not require a functional kinase domain for inducing apoptosis. The finding that the kinase activity of DAKAR is not required for apoptotic activity is consistent with similar findings reported for RIP, RIP2/RICK and RIP3.

As described in Example 9, analysis of truncated mutants of DAKAR in their capacity to induce apoptosis demonstrated that both the N-terminal kinase and C-terminal ankyrin repeat domains of DAKAR, but not DAKAR kinase activity are required to induce apoptosis and to potentiate TRAIL-induced apoptosis. Truncation mutants of muDAKAR encoding the amino terminus-proximal 1-295 amino acids (the entire kinase domain), or the carboxy terminus-proximal 525 amino acids (the entire ankyrin-repeat domain), beginning at Methionine residue 262 (DAKAR 1-295 and DAKAR 262-786). Therefore, although the kinase functionality is not required per se, other structural elements in both the kinase and non-kinase portions of the molecule may be required for its apoptotic function. Embodiments of the present invention include DAKAR polypeptides having one or more structural elements throughout the molecule, namely in the kinase and non-kinase portions that may be required for inducing apoptosis.

Overexpression of DAKAR has been shown to activate NF-κB. In one embodiment of the present invention, the requirement for an intact catalytic region for NF-κB activation may be required, which is distinct from RIP, RIP2 and RIP3, which do not require kinase activity. In alternative embodiments, version of DAKAR having a mutagenized catalytic region capable of activating NF-κB. In addition, DAKAR has been shown to be cleaved by caspase 3 and caspase 8, as described in Example 13. Therefore, embodiments of the present invention include those having intact caspase cleavage sites. Alternative embodiments comprise DAKAR mutants having one or more caspase sites altered so as to render them inactive.

DAKAR may mediate its cellular effects in cells by interacting with a number of substrates and cellular pathways. These may either be substrates that become phosphorylated, or possibly binding partners for the ankyrin repeats. In one embodiment of the present invention, a possible phosphorylation candidate is IkB and its associated subunits. In one particular embodiment, DAKAR functions to phosphorylate or form a complex with IKKα, and may function in a pathway that signals to NFkB specifically in response to developmental or differentiation cues. In alternative embodiments, this pathway does not involve inflammatory signals. These embodiments would be consistent with the observed phenotypes of IKKα^{-/-} and DAKAR^{-/-} embryos, and the signaling properties reported for IKKα^{-/-} cells. Existence of such a pathway might be analogous to reports for the IKK kinase NIK. Studies from NIK^{-/-} cells indicate that NIK does not participate in IKK activation in response to either TNF or IL-1, but seems to specifically mediate signals in response to lymphotoxin-α (LTα). Moreover, yeast two hybrid and protein interaction studies show that NIK strongly and preferentially interacts with IKKα. In alternative embodiments, IKKα as well as other components of the IKK complex (IKKβ and IKKγ) may be implicated in their ability to associate with or be phosphorylated by DAKAR. Similarly, components downstream in the signaling pathway such as the IkBs may also be implicated in DAKAR associated cellular events. For example, the ankyrin repeats of IkB are stacked helical domains which bind to the Re1 homology region (RHR) of NFkB, thereby masking its NLS. DAKAR may also bind NFkB directly in a similar manner, or perhaps to other ankyrin repeat-containing IkB proteins. DAKAR contains a putative nuclear localization site (NLS) at amino acids 469 to 482 of the polypeptide depicted in SEQ ID NO:7, and therefore is likely to undergo subcellular localization.

Furthermore, IKKα^{-/-} fibroblasts exhibit substantially lower amounts of both basal and induced NFkB DNA-binding activity than do wild-type cells, although IkB degradation and NFkB translocation is unaffected. Therefore, it appears that phosphorylation of NFkB proteins may modulate their transcriptional activity. In one embodiment of the present invention, DAKAR may be translocated to the nucleus via its nuclear localization site and function in the nucleus to directly regulate NFkB.

Other potential clues into the regulation of DAKAR come from its amino acid sequence. As previously mentioned, DAKAR contains a putative NLS, located immediately upstream of the C-terminal ankyrin repeats. Upstream of the NLS lies a consensus caspase 8 cleavage site (LALDS) at amino acids 433 to 437 of the polypeptide depicted in SEQ ID NO:7. The related kinase RIP is also cleaved by caspase 8, which has been shown to be important for TNF-induced apoptosis. DAKAR cleavage is predicted to occur at the aspartic acid residue adjacent to the serine residue (amino acid 437, or in some variants at amino acids 435-439). Other caspase substrates, including IkB, also contain a phosphorylatable serine residue adjacent to the cleavage site. Studies of IkB cleavage in vitro have demonstrated that phosphorylation at this site can influence the ability of IkB to be cleaved by caspase 3. Therefore, in certain embodiments, DAKAR may be cleaved by caspase 8 and that its cleavage may be regulated by phosphorylation. Cleavage at that site would liberate the catalytic domain of DAKAR from the NLS and the ankyrin repeats, possibly preventing nuclear localization of the catalytic domain. To address some of these possibilities, cleavage and phosphorylation site mutants of DAKAR have been generated and tested, as described in Examples 7-11 and 13. Similarly, constructs which mimic the forms of the cleaved products may also be informative with respect to the consequences of DAKAR cleavage. Studies to confirm that DAKAR is a bona fide caspase target are currently underway. As described in the nine month report, a 50 kDa band that is strongly recognized by a DAKAR polyclonal antibody has been observed in staurosporine treated 293 cells, as well as in the melanoma line WM164 and various forms of dendritic cells.

RT-PCR amplification from tissue-specific cDNA libraries was performed to detect DAKAR cDNA sequences. The results of these experiments show that DAKAR transcripts are expressed in a wide variety of human fetal and adult cells, including human brain, kidney, liver, lung, pancreas, placenta, skeletal muscle, colon, PBLs, prostate, small intestine, testis, thymus, tonsil, as well as fetal lung, liver, kidney, heart and thymus. By far the greatest expression level was seen in prostate tissue.

Methods for Making and Purifying DAKAR Polypeptides

Methods for making DAKAR polypeptides are described below. Expression, isolation, and purification of the polypeptides and fragments of the invention can be accomplished by any suitable technique, including but not limited to the following methods.

The isolated nucleic acid of the invention can be operably linked to an expression control sequence such as the pDC409 vector (Giri et al., 1990, EMBO J., 13:2821) or the derivative pDC412 vector (Wiley et al., 1995, Immunity 3:673). The pDC400 series vectors are useful for transient mammalian expression systems, such as CV-1 or 293 cells. Alternatively, the isolated nucleic acid of the invention can be linked to expression vectors such as pDC312, pDC316, or pDC317 vectors. The pDC300 series vectors all contain the SV40 origin of replication, the CMV promoter, the adenovirus tripartite leader, and the SV40 polyA and termination signals, and are useful for stable mammalian expression systems, such as CHO cells or their derivatives. Other expression control sequences and cloning technologies can also be used to produce the polypeptide recombinantly, such as the pMT2 or pED expression vectors (Kaufman et al., 1991, Nucleic Acids Res. 19:4485-4490; and Pouwels et al., 1985, Cloning Vectors: A Laboratory Manual, Elsevier, New York) and the GATEWAY Vectors (www.lifetech.com/Content/Tech-Online/molecular_biology/manuals_pps/11797016.pdf; Life Technologies; Rockville, Md.,). In the GATEWAY system the isolated nucleic acid of the invention, flanked by attB sequences, can be recombined through an integrase reaction with a GATEWAY vector such as pDONR201 containing attP sequences. This provides an entry vector for the GATEWAY system containing the isolated nucleic acid of the invention. This entry vector can be further recombined with other suitably prepared expression control sequences, such as those of the pDC400 and pDC300 series described above. Many suitable expression control sequences are known in the art. General methods of expressing recombinant polypeptides are also described in R. Kaufman, Methods in Enzymology 185:537-566 (1990). As used herein “operably linked” means that the nucleic acid of the invention and an expression control sequence are situated within a construct, vector, or cell in such a way that the polypeptide encoded by the nucleic acid is expressed when appropriate molecules (such as polymerases) are present. As one embodiment of the invention, at least one expression control sequence is operably linked to the nucleic acid of the invention in a recombinant host cell or progeny thereof, the nucleic acid and/or expression control sequence having been introduced into the host cell by transformation or transfection, for example, or by any other suitable method. As another embodiment of the invention, at least one expression control sequence is integrated into the genome of a recombinant host cell such that it is operably linked to a nucleic acid sequence encoding a polypeptide of the invention. In a further embodiment of the invention, at least one expression control sequence is operably linked to a nucleic acid of the invention through the action of a trans-acting factor such as a transcription factor, either in vitro or in a recombinant host cell.

In addition, a sequence encoding an appropriate signal peptide (native or heterologous) can be incorporated into expression vectors. The choice of signal peptide or leader can depend on factors such as the type of host cells in which the recombinant polypeptide is to be produced. To illustrate, examples of heterologous signal peptides that are functional in mammalian host cells include the signal sequence for interleukin-7 (IL-7) described in U.S. Pat. No. 4,965,195; the signal sequence for interleukin-2 receptor described in Cosman et al., Nature 312:768 (1984); the interleukin-4 receptor signal peptide described in EP 367,566; the type I interleukin-I receptor signal peptide described in U.S. Pat. No. 4,968,607; and the type II interleukin-1 receptor signal peptide described in EP 460,846. A DNA sequence for a signal peptide (secretory leader) can be fused in frame to the nucleic acid sequence of the invention so that the DNA is initially transcribed, and the mRNA translated, into a fusion polypeptide comprising the signal peptide. A signal peptide that is functional in the intended host cells is one that promotes insertion of the polypeptide into cell membranes, and most preferably, promotes extracellular secretion of the polypeptide from that host cell. The signal peptide is preferably cleaved from the polypeptide upon membrane insertion or secretion of polypeptide from the cell. The skilled artisan will also recognize that the position(s) at which the signal peptide is cleaved can differ from that predicted by computer program, and can vary according to such factors as the type of host cells employed in expressing a recombinant polypeptide. A polypeptide preparation can include a mixture of polypeptide molecules having different N-terminal amino acids, resulting from cleavage of the signal peptide at more than one site.

Established methods for introducing DNA into mammalian cells have been described (Kaufman, R. J., Large Scale Mammalian Cell Culture, 1990, pp. 15-69). Additional protocols using commercially available reagents, such as Lipofectamine lipid reagent (Gibco/BRL) or Lipofectamine-Plus lipid reagent, can be used to transfect cells (Felgner et al., Proc. Natl. Acad. Sci. USA 84:7413-7417, 1987). In addition, electroporation can be used to transfect mammalian cells using conventional procedures, such as those in Sambrook et al. (Molecular Cloning: A Laboratory Manual, 2 ed. Vol. 1-3, Cold Spring Harbor Laboratory Press, 1989). Selection of stable transformants can be performed using methods known in the art, such as, for example, resistance to cytotoxic drugs. Kaufman et al., Meth. in Enzymology 185:487-511, 1990, describes several selection schemes, such as dihydrofolate reductase (DHFR) resistance. A suitable strain for DHFR selection is CHO strain DX-B11, which is deficient in DHFR (Urlaub and Chasin, Proc. Natl. Acad. Sci. USA 77:4216-4220, 1980). A plasmid expressing the DHFR cDNA can be introduced into strain DX-B11, and only cells that contain the plasmid can grow in the appropriate selective media. Other examples of selectable markers that can be incorporated into an expression vector include cDNAs conferring resistance to antibiotics, such as G418 and hygromycin B. Cells harboring the vector can be selected on the basis of resistance to these compounds.

Alternatively, DAKAR gene products can be obtained via homologous recombination, or “gene targeting,” techniques. Such techniques employ the introduction of exogenous transcription control elements (such as the CMV promoter or the like) in a particular predetermined site on the genome, to induce expression of the endogenous nucleic acid sequence of interest (see, for example, U.S. Pat. No. 5,272,071). The location of integration into a host chromosome or genome can be easily determined by one of skill in the art, given the known location and sequence of the gene. In a preferred embodiment, the present invention also contemplates the introduction of exogenous transcriptional control elements in conjunction with an amplifiable gene, to produce increased amounts of the gene product, again, without the need for isolation of the gene sequence itself from the host cell.

A number of types of cells can act as suitable host cells for expression of the polypeptide. Mammalian host cells include, for example, the COS-7 line of monkey kidney cells (ATCC CRL 1651) (Gluzman et al., Cell 23:175, 1981), L cells, C127 cells, 3T3 cells (ATCC CCL 163), Chinese hamster ovary (CHO) cells or their derivatives such as Veggie CHO and related cell lines which grow in serum-free media (Rasmussen et al., 1998, Cytotechnology 28:31), HeLa cells, BHK (ATCC CRL 10) cell lines, the CV1/EBNA cell line derived from the African green monkey kidney cell line CV1 (ATCC CCL 70) (McMahan et al., 1991, EMBO J 10:2821, 1991), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. Optionally, mammalian cell lines such as HepG2/3B, KB, NIH 3T3 or S49, for example, can be used for expression of the polypeptide when it is desirable to use the polypeptide in various signal transduction or reporter assays. Alternatively, it is possible to produce the polypeptide in lower eukaryotes such as yeast or in prokaryotes such as bacteria. Suitable yeast include Saccharomyces cerevisiae, Schizosaccharomyces pombe, Kluyveromyces strains, Candida, or any yeast strain capable of expressing heterologous polypeptides. Suitable bacterial strains include Escherichia coli, Bacillus subtilis, Salmonella typhimurium, or any bacterial strain capable of expressing heterologous polypeptides. If the polypeptide is made in yeast or bacteria, it may be desirable to modify the polypeptide produced therein, for example by phosphorylation or glycosylation of the appropriate sites, in order to obtain the functional polypeptide. Such covalent attachments can be accomplished using known chemical or enzymatic methods. The polypeptide can also be produced by operably linking the isolated nucleic acid of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac® kit), and such methods are well known in the art, as described in Summers and Smith, Texas Agricultural Experiment Station Bulletin No. 1555 (1987), and Luckow and Summers, Bio/Technology 6:47 (1988). Cell-free translation systems could also be employed to produce polypeptides using RNAs derived from nucleic acid constructs disclosed herein. A host cell that comprises an isolated nucleic acid of the invention, preferably operably linked to at least one expression control sequence, is a “recombinant host cell”.

The polypeptide of the invention can be prepared by culturing transformed host cells under culture conditions suitable to express the recombinant polypeptide. The resulting expressed polypeptide can then be purified from such culture (i.e., from culture medium or cell extracts) using known purification processes, such as selective precipitation with various salts, gel filtration, and ion exchange chromatography. The purification of the polypeptide can also include an affinity column containing agents which will bind to the polypeptide; one or more column steps over such affinity resins as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography using an antibody that specifically binds one or more DAKAR epitopes. Alternatively, the polypeptide of the invention can also be expressed in a form which will facilitate purification. For example, it can be expressed as a fusion polypeptide, that is, it may be fused with maltose binding polypeptide (MBP), glutathione-S-transferase (GST), thioredoxin (TRX), a polyHis peptide, and/or fragments thereof. Kits for expression and purification of such fusion polypeptides are commercially available from New England BioLabs (Beverly, Mass.), Pharmacia (Piscataway, N.J.) and InVitrogen, respectively. The polypeptide can also be tagged with an epitope and subsequently purified by using a specific antibody directed to such epitope. One such epitope (FLAG®) is commercially available from Kodak (New Haven, Conn.). Finally, one or more reverse-phase high performance liquid chromatography (RP-HPLC) steps employing hydrophobic RP-HPLC media, e.g., silica gel having pendant methyl or other aliphatic groups, can be employed to further purify the polypeptide. Some or all of the foregoing purification steps, in various combinations, can also be employed to provide a substantially homogeneous isolated recombinant polypeptide. The polypeptide thus purified is substantially free of other mammalian polypeptides and is defined in accordance with the present invention as an “isolated polypeptide”; such isolated polypeptides of the invention include isolated antibodies that bind to DAKAR polypeptides, fragments, variants, binding partners etc. The polypeptide of the invention can also be expressed as a product of transgenic animals, e.g., as a component of the milk of transgenic cows, goats, pigs, or sheep which are characterized by somatic or germ cells containing a nucleotide sequence encoding the polypeptide.

It is also possible to utilize an affinity column comprising a polypeptide-binding polypeptide of the invention, such as a monoclonal antibody generated against polypeptides of the invention, to affinity-purify expressed polypeptides. These polypeptides can be removed from an affinity column using conventional techniques, e.g., in a high salt elution buffer and then dialyzed into a lower salt buffer for use or by changing pH or other components depending on the affinity matrix utilized, or be competitively removed using the naturally occurring substrate of the affinity moiety, such as a polypeptide derived from the invention. In this aspect of the invention, polypeptide-binding polypeptides, such as the anti-polypeptide antibodies of the invention or other polypeptides that can interact with the polypeptide of the invention, can be bound to a solid phase support such as a column chromatography matrix or a similar substrate suitable for identifying, separating, or purifying cells that express polypeptides of the invention on their surface. Adherence of polypeptide-binding polypeptides of the invention to a solid phase contacting surface can be accomplished by any means, for example, magnetic microspheres can be coated with these polypeptide-binding polypeptides and held in the incubation vessel through a magnetic field. Suspensions of cell mixtures are contacted with the solid phase that has such polypeptide-binding polypeptides thereon. Cells having polypeptides of the invention on their surface bind to the fixed polypeptide-binding polypeptide and unbound cells then are washed away. This affinity-binding method is useful for purifying, screening, or separating such polypeptide-expressing cells from solution. Methods of releasing positively selected cells from the solid phase are known in the art and encompass, for example, the use of enzymes. Such enzymes are preferably non-toxic and non-injurious to the cells and are preferably directed to cleaving the cell-surface binding partner. Alternatively, mixtures of cells suspected of containing polypeptide-expressing cells of the invention first can be incubated with a biotinylated polypeptide-binding polypeptide of the invention. The resulting mixture then is passed through a column packed with avidin-coated beads, whereby the high affinity of biotin for avidin provides the binding of the polypeptide-binding cells to the beads. Use of avidin-coated beads is known in the art. See Berenson, et al. J. Cell. Biochem., 10D:239 (1986). Wash of unbound material and the release of the bound cells is performed using conventional methods.

The polypeptide can also be produced by known conventional chemical synthesis. Methods for constructing the polypeptides of the present invention by synthetic means are known to those skilled in the art. The synthetically-constructed polypeptide sequences, by virtue of sharing primary, secondary or tertiary structural and/or conformational characteristics with DAKAR polypeptides can possess biological properties in common therewith, including DAKAR polypeptide activity. Thus, they can be employed as biologically active or immunological substitutes for natural, purified polypeptides in screening of therapeutic compounds and in immunological processes for the development of antibodies.

The desired degree of purity depends on the intended use of the polypeptide. A relatively high degree of purity is desired when the polypeptide is to be administered in vivo, for example. In such a case, the polypeptides are purified such that no polypeptide bands corresponding to other polypeptides are detectable upon analysis by SDS-polyacrylamide gel electrophoresis (SDS-PAGE). It will be recognized by one skilled in the pertinent field that multiple bands corresponding to the polypeptide can be visualized by SDS-PAGE, due to differential glycosylation, differential post-translational processing, and the like. Most preferably, the polypeptide of the invention is purified to substantial homogeneity, as indicated by a single polypeptide band upon analysis by SDS-PAGE. The polypeptide band can be visualized by silver staining, Coomassie blue staining, or (if the polypeptide is radiolabeled) by autoradiography.

Methods of Use for DAKAR Polynucleotides, Polypeptides, Agonists and Antagonists

Typical biological activities or functions associated with DAKAR polypeptides include, for example, apoptosis, cellular differentiation, proliferation, cell cycle and/or senescence. In particular embodiments, DAKAR may selectively influence epithelial apoptosis, cellular differentiation, proliferation, cell cycle and/or senescence, as well as keratinocyte apoptosis, cellular differentiation, proliferation, cell cycle and/or senescence. DAKAR polypeptides having kinase activity phosphorylate other molecules. The kinase activity is associated with the catalytic or kinase domain of DAKAR polypeptides. Thus, for uses requiring kinase activity, preferred DAKAR polypeptides include those having the kinase domain and exhibiting the capacity to phosphorylate other proteins or in alternative embodiments, to auto-phosphorylate.

Any method which neutralizes DAKAR polypeptides and/or inhibits expression of the DAKAR genes (either transcription or translation) can be used to reduce the biological activities of DAKAR polypeptides. In particular embodiments, antagonists inhibit the binding of at least one DAKAR polypeptide to cells, thereby inhibiting biological activities induced by the binding of those DAKAR polypeptides to the cells. In certain other embodiments of the invention, antagonists can be designed to reduce the level of endogenous DAKAR gene expression, e.g., using well-known antisense or ribozyme approaches to inhibit or prevent translation of DAKAR mRNA transcripts; triple helix approaches to inhibit transcription of DAKAR family genes; or targeted homologous recombination to inactivate or “knock out” the DAKAR genes or their endogenous promoters or enhancer elements. Such antisense, ribozyme, and triple helix antagonists can be designed to reduce or inhibit either unimpaired, or if appropriate, mutant DAKAR gene activity. Techniques for the production and use of such molecules are well known to those of skill in the art.

Antisense RNA and DNA molecules act to directly block the translation of mRNA by hybridizing to targeted mRNA and preventing polypeptide translation. Antisense approaches involve the design of oligonucleotides (either DNA or RNA) that are complementary to a DAKAR mRNA. The antisense oligonucleotides will bind to the complementary target gene mRNA transcripts and prevent translation. Absolute complementarity, although preferred, is not required. A sequence “complementary” to a portion of a nucleic acid, as referred to herein, means a sequence having sufficient complementarity to be able to hybridize with the nucleic acid, forming a stable duplex (or triplex, as appropriate). In the case of double-stranded antisense nucleic acids, a single strand of the duplex DNA can thus be tested, or triplex formation can be assayed. The ability to hybridize will depend on both the degree of complementarity and the length of the antisense nucleic acid. Preferred oligonucleotides are complementary to the 5′ end of the message, e.g., the 5′ untranslated sequence up to and including the AUG initiation codon. However, oligonucleotides complementary to the 5′- or 3′-non-translated, non-coding regions of the DAKAR gene transcript, or to the coding regions, could be used in an antisense approach to inhibit translation of endogenous DAKAR mRNA. Antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides. The oligonucleotides can be DNA or RNA or chimeric mixtures or derivatives or modified versions thereof, single-stranded or double-stranded. Chimeric oligonucleotides, oligonucleosides, or mixed oligonucleotides/oligonucleosides of the invention can be of several different types. These include a first type wherein the “gap” segment of nucleotides is positioned between 5′ and 3′ “wing” segments of linked nucleosides and a second “open end” type wherein the “gap” segment is located at either the 3′ or the 5′ terminus of the oligomeric compound (see, e.g., U.S. Pat. No. 5,985,664). Oligonucleotides of the first type are also known in the art as “gapmers” or gapped oligonucleotides. Oligonucleotides of the second type are also known in the art as “hemimers” or “wingmers”. The oligonucleotide can be modified at the base moiety, sugar moiety, or phosphate backbone, for example, to improve stability of the molecule, hybridization, etc. The oligonucleotide can include other appended groups such as peptides (e.g., for targeting host cell receptors in vivo), or agents facilitating transport across the cell membrane (see, e.g., Letsinger et al., 1989, Proc Natl Acad Sci USA 86:6553-6556; Lemaitre et al., 1987, Proc Natl Acad Sci 84:648-652; PCT Publication No. WO88/09810), or hybridization-triggered cleavage agents or intercalating agents. (See, e.g., Zon, 1988, Pharm. Res. 5:539-549). The antisense molecules should be delivered to cells which express the DAKAR transcript in vivo. A number of methods have been developed for delivering antisense DNA or RNA to cells; e.g., antisense molecules can be injected directly into the tissue or cell derivation site, or modified antisense molecules, designed to target the desired cells (e.g., antisense linked to peptides or antibodies that specifically bind receptors or antigens expressed on the target cell surface) can be administered systemically. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous DAKAR gene transcripts and thereby prevent translation of the DAKAR mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells.

Ribozyme molecules designed to catalytically cleave DAKAR mRNA transcripts can also be used to prevent translation of DAKAR mRNA and expression of DAKAR polypeptides. (See, e.g., PCT International Publication WO90/11364 and U.S. Pat. No. 5,824,519). The ribozymes that can be used in the present invention include hammerhead ribozymes (Haseloff and Gerlach, 1988, Nature, 334:585-591), RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (International Patent Application No. WO 88/04300; Been and Cech, 1986, Cell, 47:207-216). As in the antisense approach, the ribozymes can be composed of modified oligonucleotides (e.g. for improved stability, targeting, etc.) and should be delivered to cells which express the DAKAR polypeptide in vivo. A preferred method of delivery involves using a DNA construct “encoding” the ribozyme under the control of a strong constitutive pol III or pol II promoter, so that transfected cells will produce sufficient quantities of the ribozyme to destroy endogenous DAKAR messages and inhibit translation. Because ribozymes, unlike antisense molecules, are catalytic, a lower intracellular concentration is required for efficiency.

Alternatively, endogenous DAKAR gene expression can be reduced by targeting deoxyribonucleotide sequences complementary to the regulatory region of the target gene (i.e., the target gene promoter and/or enhancers) to form triple helical structures that prevent transcription of the target DAKAR gene. (See generally, Helene, 1991, Anticancer Drug Des., 6(6):569-584; Helene et al., 1992, Ann. N.Y. Acad. Sci., 660:27-36; and Maher, 1992, Bioassays 14(12):807-815).

Anti-sense RNA and DNA, ribozyme, and triple helix molecules of the invention can be prepared by any method known in the art for the synthesis of DNA and RNA molecules. These include techniques for chemically synthesizing oligodeoxyribonucleotides and oligoribonucleotides well known in the art such as for example solid phase phosphoramidite chemical synthesis. Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al., 1988, Nucl. Acids Res. 16:3209. Methylphosphonate oligonucleotides can be prepared by use of controlled pore glass polymer supports (Sarin et al., 1988, Proc. Natl. Acad. Sci. USA 85:7448-7451). Alternatively, RNA molecules can be generated by in vitro and in vivo transcription of DNA sequences encoding the antisense RNA molecule. Such DNA sequences can be incorporated into a wide variety of vectors that incorporate suitable RNA polymerase promoters such as the T7 or SP6 polymerase promoters. Alternatively, antisense cDNA constructs that synthesize either constitutive or inducible antisense RNA, depending on the promoter used, can be introduced stably into cell lines.

Knockout Mice

Endogenous target gene expression can also be reduced by inactivating or “knocking out” the target gene or its promoter using targeted homologous recombination (e.g., see Smithies, et al., 1985, Nature 317:230-234; Thomas and Capecchi, 1987, Cell 51:503-512; Thompson, et al., 1989, Cell 5:313-321). For example, a mutant, non-functional target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target gene in vivo. Insertion of the DNA construct, via targeted homologous recombination, results in inactivation of the target gene. Such approaches are particularly suited in the agricultural field where modifications to ES (embryonic stem) cells can be used to generate animal offspring with an inactive target gene (e.g., see Thomas and Capecchi, 1987 and Thompson, 1989, supra), or in model organisms such as Caenorhabditis elegans where the “RNA interference” (“RNAi”) technique (Grishok, Tabara, and Mello, 2000, Genetic requirements for inheritance of RNAi in C. elegans, Science 287(5462):2494-2497), or the introduction of transgenes (Dernburg et al., 2000, Transgene-mediated cosuppression in the C. elegans germ line, Genes Dev. 14(13):1578-1583) are used to inhibit the expression of specific target genes. However this approach can be adapted for use in humans provided the recombinant DNA constructs are directly administered or targeted to the required site in vivo using appropriate vectors such as viral vectors.

DAKAR deficient mice were generated by gene targeting in embryonic stem cells using standard techniques, such as those described in the MOUSE KIT™ by Lexicon Genetics Inc. (The Woodlands, Tex.). Methods and techniques for the generation of knockout mice are well known in the art (see, for example, Ramirez-Soltis, R., et al., Nature 378:720-724, 1995 and Hogan, B., et al., Manipulating the Mouse Embryo: A Laboratory Manual, Cold Spring Harbor Laboratory, 1994; and in particular, the Lexicon MOUSE KIT™ Instruction Manual). A DAKAR gene targeting construct was prepared using conventional techniques, such as those described in Hasty, P., et al., “Gene targeting vectors for mammalian cells” in Gene Targeting: a Practical Approach, A. L. Joyner, ed. IRL Press: Oxford, pp 1-31, (1993). Specifically, DAKAR^{-/-} mice were prepared as described in Example 12. Mice lacking DAKAR display a specific defect in keratinocyte differentiation, proliferation and/or other related pathway that affects all squamous epithelia and results in perinatal lethality. Genotyping identified a homozygous mutant, suggesting that DAKAR^{-/-} pups were dying just prior to or after birth. Grossly, all e17.5 and e18.5 DAKAR mutants have thickened non-wrinkled skin, poorly defined oral and auricular orifices, and the tail is fused to the skin in the area of the rectum and urethra opening resulting in no visible rectal or urethra orifice (anal and urethra atresia). Atresia is defined as the congenital absence or closure of a normal body orifice or tubular organ. Additionally, there was a high degree of atresia surrounding the mouth and ears of the mutant embryos. Although a ridge line was evident at the site of the mouth, this ridge was impenetrable. In contrast, wild-type embryos at this stage have open mouths with well-developed snouts and whiskers. Moreover, the limbs appeared rigid and somewhat stunted and in some embryos the toes appear to be fused. Finally, there is a linear red friable streak along the dorsal midline of e18.5 and newborn mutants.

Microscopically, e17.5 and e18.5 DAKAR^{-/-} mutants consistently have markedly thickened corneal epithelium of the surface epidermis compared to wild-type embryos. This hyperplasia of the suprabasal layer may explain the tautness and lack of skin folds seen in the embryos. Moreover, the terminally differentiated outermost skin layer, the stratum corneum, which gives the ridged appearance of the skin surface, appears to be absent in the mutant skin. The epidermis at dorsal midline in e18.5 and newborn mutants is thin, with degenerating cells in most layers of the epidermis. In contrast, the epidermis not at midline in the e18.5 and newborn mutants is thickened. The corneal epithelium between the digits of all four feet is partially fused. In addition, the corneal epithelium of the oral cavity and lips (oral atresia), esophagus (esophageal atresia), anterior portion of the stomach (atresia of the anterior stomach) are fused. Dissection of mutant embryos has indicated that all major organs are present, including lungs, liver, heart, kidneys, thymus, spleen, stomach, colon, and testes. Additionally, the urinary bladders are extremely full and distended, which is consistent with the urethra and anal atresia observed as a result of increased proliferation and/or lack of differentiation of cells around these and other orifices.

The phenotype observed for DAKAR^{-/-} embryos shows similarities to that reported for IKKα knockout animals (Takeda, K., et al., Science, 284:313, 1999; Hu, Y., et al., Science, 284:316, 1999; Hu, Y., et al., Nature, 410:710, 2001). IKKα is a component of a multi-protein complex made up of two serine/threonine kinases, IKKα and IKKβ, as well as a regulatory subunit, IKKγ. IKKα and IKKβ can form homo- and hetero-dimers and tightly associate with dimers or trimers of IKKγ and phosphorylate IkB. IkB phosphorylation targets it for degradation, thereby allowing nuclear translocation and activation of NFκB. IKKα^{-/-} mice die perinatally and display highly dysregulated epidermal differentiation. In these mice, the suprabasal skin layer has overproliferated and appears thickened, while the outermost layers, the stratum granulosum and the stratum corneum, are completely absent. The limbs of IKKα^{-/-} mice are severely compacted, more than in DAKAR^{-/-} embryos. IKKα^{-/-} mice do not have a fused mouth, but they do display closure of the esophagus, possibly as a result of increased adhesiveness of mutant epidermis. Surprisingly, activation of NFκB in response to inflammatory stimuli is unperturbed in IKKα^{-/-} cells. This is not apparently not a consequence of functional redundancy between IKKα and IKKβ, because IKKα^{-/-} cells exhibit marked defects in NFkB activation in response to TNF or IL-1. This strongly suggests that the role of IKKα in NFkB signaling is in regulating morphogenetic events, particularly keratinocyte proliferation and differentiation, not in regulating inflammatory responses. These results are consistent with expression of dominant negative NFkB inhibitory proteins in transgenic mice, which also results in epidermal hyperplasia. Several keratin genes have been shown to be regulated by NFkB, including K5, K6 and K14. The expression of keratin K5 and K14 is restricted to the basal layers of healthy epidermis and are suppressed by NFkB. In contrast, K6 is induced by NFkB, and is expressed in response to proliferative and inflammatory stimuli, and marks keratinocyte activation. Thus, in different epidermal layers, NFkB may exert either positive or negative effects on proliferation and differentiation. The related phenotypes between DAKAR^{-/-} and □□□□^{-/-} suggest that these molecules may function together in a pathway leading to NFkB activation in keratinocytes. In one embodiment of the present invention, the consequence of NFkB activation in skin may be associated with inducing and maintaining cells in a terminally differentiated state as they migrate outward and eventually die.

Organisms that have enhanced, reduced, or modified expression of the gene(s) corresponding to the nucleic acid sequences disclosed herein are provided. The desired change in gene expression can be achieved through the use of antisense nucleic acids or ribozymes that bind and/or cleave the mRNA transcribed from the gene (Albert and Morris, 1994, Trends Pharmacol. Sci. 15(7):250-254; Lavarosky et al., 1997, Biochem. Mol. Med. 62(1):11-22; and Hampel, 1998, Prog. Nucleic Acid Res. Mol. Biol. 58:1-39). Transgenic animals that have multiple copies of the gene(s) corresponding to the nucleic acid sequences disclosed herein, preferably produced by transformation of cells with genetic constructs that are stably maintained within the transformed cells and their progeny, are provided. Transgenic animals that have modified genetic control regions that increase or reduce gene expression levels, or that change temporal or spatial patterns of gene expression, are also provided (see European Patent No. 0 649 464 B1). In addition, organisms are provided in which the gene(s) corresponding to the nucleic acid sequences disclosed herein have been partially or completely inactivated, through insertion of extraneous sequences into the corresponding gene(s) or through deletion of all or part of the corresponding gene(s). Partial or complete gene inactivation can be accomplished through insertion, preferably followed by imprecise excision, of transposable elements (Plasterk, 1992, Bioessays 14(9):629-633; Zwaal et al., 1993, Proc. Natl. Acad. Sci. USA 90(16):7431-7435; Clark et al., 1994, Proc. Natl. Acad. Sci. USA 91(2):719-722), or through homologous recombination, preferably detected by positive/negative genetic selection strategies (Mansour et al., 1988, Nature 336:348-352; U.S. Pat. Nos. 5,464,764; 5,487,992; 5,627,059; 5,631,153; 5,614,396; 5,616,491; and 5,679,523). These organisms with altered gene expression are preferably eukaryotes and more preferably are mammals. Such organisms are useful for the development of non-human models for the study of disorders involving the corresponding gene(s), and for the development of assay systems for the identification of molecules that interact with the polypeptide product(s) of the corresponding gene(s).

Also encompassed within the invention are DAKAR polypeptide variants with partner binding sites that have been altered in conformation so that (1) the DAKAR variant will still bind to its partner(s), but a specified small molecule will fit into the altered binding site and block that interaction, or (2) the DAKAR variant will no longer bind to its partner(s) unless a specified small molecule is present (see for example Bishop et al., 2000, Nature 407:395-401). Nucleic acids encoding such altered DAKAR polypeptides can be introduced into organisms according to methods described herein, and can replace the endogenous nucleic acid sequences encoding the corresponding DAKAR polypeptide. Such methods allow for the interaction of a particular DAKAR polypeptide with its binding partners to be regulated by administration of a small molecule compound to an organism, either systemically or in a localized manner.

The DAKAR polypeptides themselves can also be employed in inhibiting a biological activity of DAKAR in in vitro or in vivo procedures. Encompassed within the invention are mutated DAKAR polypeptides, such as those described in the Examples below wherein, for example, the kinase domain has been inactivated, and therefore act as “dominant negative” inhibitors of native DAKAR polypeptide function when expressed as fragments or as components of fusion polypeptides. Alternatively, the present invention may employ DAKAR mutants having mutations to one or more phosphorylation sites, nuclear localization site(s), ankyrin repeats and/or other functional domains of DAKAR. For example, a purified polypeptide domain of the present invention can be used to inhibit binding of DAKAR polypeptides to endogenous binding partners. Such use effectively would block DAKAR polypeptide interactions and inhibit DAKAR polypeptide activities. In still another aspect of the invention, a soluble form of the DAKAR binding partner, which is expressed on //cell types of binding partners// is used to bind to, and competitively inhibit, activation of the endogenous DAKAR polypeptide. Furthermore, antibodies which bind to DAKAR polypeptides often inhibit DAKAR polypeptide activity and act as antagonists. For example, antibodies that specifically recognize one or more epitopes of DAKAR polypeptides, or epitopes of conserved variants of DAKAR polypeptides, or peptide fragments of the DAKAR polypeptide can be used in the invention to inhibit DAKAR polypeptide activity. Such antibodies include but are not limited to polyclonal antibodies, monoclonal antibodies (mAbs), humanized or chimeric antibodies, single chain antibodies, Fab fragments, F(ab′)2 fragments, fragments produced by a Fab expression library, anti-idiotypic (anti-Id) antibodies, and epitope-binding fragments of any of the above. Alternatively, purified and modified DAKAR polypeptides of the present invention can be administered to modulate interactions between DAKAR polypeptides and DAKAR binding partners that are not membrane-bound. Such an approach will allow an alternative method for the modification of DAKAR-influenced bioactivity.

In an alternative aspect, the invention further encompasses the use of agonists of DAKAR polypeptide activity to treat or ameliorate the symptoms of a disease for which increased DAKAR polypeptide activity is beneficial. Such diseases include but are not limited to <disease1>and <disease2>. In a preferred aspect, the invention entails administering compositions comprising an DAKAR nucleic acid or an DAKAR polypeptide to cells in vitro, to cells ex vivo, to cells in vivo, and/or to a multicellular organism such as a vertebrate or mammal. Preferred therapeutic forms of DAKAR are soluble forms, as described above. In still another aspect of the invention, the compositions comprise administering a DAKAR-encoding nucleic acid for expression of a DAKAR polypeptide in a host organism for treatment of disease. Particularly preferred in this regard is expression in a human patient for treatment of a dysfunction associated with aberrant (e.g., decreased) endogenous activity of a DAKAR family polypeptide. Furthermore, the invention encompasses the administration to cells and/or organisms of compounds found to increase the endogenous activity of DAKAR polypeptides. One example of compounds that increase DAKAR polypeptide activity are agonistic antibodies, preferably monoclonal antibodies, that bind to DAKAR polypeptides and/or binding partners, which may increase DAKAR polypeptide activity by causing constitutive intracellular signaling (or “ligand mimicking”), or by preventing the binding of a native inhibitor of DAKAR polypeptide activity.

Antibodies to DAKAR Polypeptides

Antibodies that are immunoreactive with the polypeptides of the invention are provided herein. Such antibodies specifically bind to the polypeptides via the antigen-binding sites of the antibody (as opposed to non-specific binding). In the present invention, specifically binding antibodies are those that will specifically recognize and bind with DAKAR polypeptides, homologues, and variants, but not with other molecules. In one preferred embodiment, the antibodies are specific for the polypeptides of the present invention and do not cross-react with other polypeptides. In this manner, the DAKAR polypeptides, fragments, variants, fusion polypeptides, etc., as set forth above can be employed as “immunogens” in producing antibodies immunoreactive therewith.

More specifically, the polypeptides, fragment, variants, fusion polypeptides, etc. contain antigenic determinants or epitopes that elicit the formation of antibodies. These antigenic determinants or epitopes can be either linear or conformational (discontinuous). Linear epitopes are composed of a single section of amino acids of the polypeptide, while conformational or discontinuous epitopes are composed of amino acids sections from different regions of the polypeptide chain that are brought into close proximity upon polypeptide folding (Janeway and Travers, Immuno Biology 3:9 (Garland Publishing Inc., 2nd ed. 1996)). Because folded polypeptides have complex surfaces, the number of epitopes available is quite numerous; however, due to the conformation of the polypeptide and steric hindrances, the number of antibodies that actually bind to the epitopes is less than the number of available epitopes (Janeway and Travers, Immuno Biology 2:14 (Garland Publishing Inc., 2nd ed. 1996)). Epitopes can be identified by any of the methods known in the art. Thus, one aspect of the present invention relates to the antigenic epitopes of the polypeptides of the invention. Such epitopes are useful for raising antibodies, in particular monoclonal antibodies, as described in more detail below. Additionally, epitopes from the polypeptides of the invention can be used as research reagents, in assays, and to purify specific binding antibodies from substances such as polyclonal sera or supernatants from cultured hybridomas. Such epitopes or variants thereof can be produced using techniques well known in the art such as solid-phase synthesis, chemical or enzymatic cleavage of a polypeptide, or using recombinant DNA technology.

As to the antibodies that can be elicited by the epitopes of the polypeptides of the invention, whether the epitopes have been isolated or remain part of the polypeptides, both polyclonal and monoclonal antibodies can be prepared by conventional techniques. See, for example, Monoclonal Antibodies, Hybridomas: A New Dimension in Biological Analyses, Kennet et al. (eds.), Plenum Press, New York (1980); and Antibodies: A Laboratory Manual, Harlow and Land (eds.), Cold Spring Harbor Laboratory Press, Cold Spring Harbor, N.Y., (1988); Kohler and Milstein, (U.S. Pat. No. 4,376,110); the human B-cell hybridoma technique (Kozbor et al., 1984, J. Immunol. 133:3001-3005; Cole et al., 1983, Proc. Natl. Acad. Sci. USA 80:2026-2030); and the EBV-hybridoma technique (Cole et al., 1985, Monoclonal Antibodies And Cancer Therapy, Alan R. Liss, Inc., pp. 77-96). Hybridoma cell lines that produce monoclonal antibodies specific for the polypeptides of the invention are also contemplated herein. Such hybridomas can be produced and identified by conventional techniques. The hybridoma producing the mAb of this invention can be cultivated in vitro or in vivo. Production of high titers of mAbs in vivo makes this the presently preferred method of production. One method for producing such a hybridoma cell line comprises immunizing an animal with a polypeptide; harvesting spleen cells from the immunized animal; fusing said spleen cells to a myeloma cell line, thereby generating hybridoma cells; and identifying a hybridoma cell line that produces a monoclonal antibody that binds the polypeptide. For the production of antibodies, various host animals can be immunized by injection with one or more of the following: a DAKAR polypeptide, a fragment of a DAKAR polypeptide, a functional equivalent of a DAKAR polypeptide, or a mutant form of a DAKAR polypeptide. Such host animals can include but are not limited to rabbits, guinea pigs, mice, and rats. Various adjuvants can be used to increase the immunologic response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjutants such as BCG (bacille Calmette-Guerin) and Corynebacterium parvum. The monoclonal antibodies can be recovered by conventional techniques. Such monoclonal antibodies can be of any immunoglobulin class including IgG, IgM, IgE, IgA, IgD and any subclass thereof.

In addition, techniques developed for the production of “chimeric antibodies” (Takeda et al., 1985, Nature, 314:452-454; Morrison et al., 1984, Proc Natl Acad Sci USA 81:6851-6855; Boulianne et al., 1984, Nature 312:643-646; Neuberger et al., 1985, Nature 314:268-270) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a porcine mAb and a human immunoglobulin constant region. The monoclonal antibodies of the present invention also include humanized versions of murine monoclonal antibodies. Such humanized antibodies can be prepared by known techniques and offer the advantage of reduced immunogenicity when the antibodies are administered to humans. In one embodiment, a humanized monoclonal antibody comprises the variable region of a murine antibody (or just the antigen binding site thereof) and a constant region derived from a human antibody. Alternatively, a humanized antibody fragment can comprise the antigen binding site of a murine monoclonal antibody and a variable region fragment (lacking the antigen-binding site) derived from a human antibody. Procedures for the production of chimeric and further engineered monoclonal antibodies include those described in Riechmann et al. (Nature 332:323, 1988), Liu et al. (PNAS 84:3439, 1987), Larrick et al. (Bio/Technology 7:934, 1989), and Winter and Harris (TIPS 14:139, Can, 1993). Useful techniques for humanizing antibodies are also discussed in U.S. Pat. No. 6,054,297. Procedures to generate antibodies transgenically can be found in GB 2,272,440, U.S. Pat. Nos. 5,569,825 and 5,545,806, and related patents. Preferably, for use in humans, the antibodies are human or humanized; techniques for creating such human or humanized antibodies are also well known and are commercially available from, for example, Medarex Inc. (Princeton, N.J.) and Abgenix Inc. (Fremont, Calif.). In another preferred embodiment, fully human antibodies for use in humans are produced by screening a phage display library of human antibody variable domains (Vaughan et al., 1998, Nat Biotechnol. 16(6):535-539; and U.S. Pat. No. 5,969,108).

Antigen-binding antibody fragments that recognize specific epitopes can be generated by known techniques. For example, such fragments include but are not limited to: the F(ab′)2 fragments which can be produced by pepsin digestion of the antibody molecule and the Fab fragments which can be generated by reducing the disulfide bridges of the (ab′)2 fragments. Alternatively, Fab expression libraries can be constructed (Huse et al., 1989, Science, 246:1275-1281) to allow rapid and easy identification of monoclonal Fab fragments with the desired specificity. Techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can also be adapted to produce single chain antibodies against DAKAR gene products. Single chain antibodies are formed by linking the heavy and light chain fragments of the Fv region via an amino acid bridge, resulting in a single chain polypeptide. Such single chain antibodies can also be useful intracellularly (i.e., as ‘intrabodies), for example as described by Marasco et al. (J Immunol. Methods 231:223-238, 1999) for genetic therapy in HIV infection. In addition, antibodies to the DAKAR polypeptide can, in turn, be utilized to generate anti-idiotype antibodies that “mimic” the DAKAR polypeptide and that may bind to the DAKAR polypeptide's binding partners using techniques well known to those skilled in the art. (See, e.g., Greenspan & Bona, 1993, FASEB J7(5):437-444; and Nissinoff, 1991, J. Immunol. 147(8):2429-2438).

Antibodies that are immunoreactive with the polypeptides of the invention include bispecific antibodies (i.e., antibodies that are immunoreactive with the polypeptides of the invention via a first antigen binding domain, and also immunoreactive with a different polypeptide via a second antigen binding domain). A variety of bispecific antibodies have been prepared, and found useful both in vitro and in vivo (see, for example, U.S. Pat. No. 5,807,706; and Cao and Suresh, 1998, Bioconjugate Chem 9:635-644). Numerous methods of preparing bispecific antibodies are known in the art, including the use of hybrid-hybridomas such as quadromas, which are formed by fusing two differed hybridomas, and triomas, which are formed by fusing a hybridoma with a lymphocyte (Milstein and Cuello, 1983, Nature 305:537-540; U.S. Pat. No. 4,474,893; and U.S. Pat. No. 6,106,833). U.S. Pat. No. 6,060,285 discloses a process for the production of bispecific antibodies in which at least the genes for the light chain and the variable portion of the heavy chain of an antibody having a first specificity are transfected into a hybridoma cell secreting an antibody having a second specificity. Chemical coupling of antibody fragments has also been used to prepare antigen-binding molecules having specificity for two different antigens (Brennan et al., 1985, Science 229:81-83; Glennie et al., J Immunol., 1987, 139:2367-2375; and U.S. Pat. No. 6,010,902). Bispecific antibodies can also be produced via recombinant means, for example, by using. the leucine zipper moieties from the Fos and Jun proteins (which preferentially form heterodimers) as described by Kostelny et al. (J. Immunol. 148:1547-4553; 1992). U.S. Pat. No. 5,582,996 discloses the use of complementary interactive domains (such as leucine zipper moieties or other lock and key interactive domain structures) to facilitate heterodimer formation in the production of bispecific antibodies. Tetravalent, bispecific molecules can be prepared by fusion of DNA encoding the heavy chain of an F(ab′)2 fragment of an antibody with either DNA encoding the heavy chain of a second F(ab′)2 molecule (in which the CH1 domain is replaced by a CH3 domain), or with DNA encoding a single chain FV fragment of an antibody, as described in U.S. Pat. No. 5,959,083. Expression of the resultant fusion genes in mammalian cells, together with the genes for the corresponding light chains, yields tetravalent bispecific molecules having specificity for selected antigens. Bispecific antibodies can also be produced as described in U.S. Pat. No. 5,807,706. Generally, the method involves introducing a protuberance (constructed by replacing small amino acid side chains with larger side chains) at the interface of a first polypeptide and a corresponding cavity (prepared by replacing large amino acid side chains with smaller ones) in the interface of a second polypeptide. Moreover, single-chain variable fragments (sFvs) have been prepared by covalently joining two variable domains; the resulting antibody fragments can form dimers or trimers, depending on the length of a flexible linker between the two variable domains (Kortt et al., 1997, Protein Engineering 10:423-433).

Screening procedures by which such antibodies can be identified are well known, and can involve immunoaffinity chromatography, for example. Antibodies can be screened for agonistic (i.e., ligand-mimicking) properties. Such antibodies, upon binding to cell surface DAKAR, induce biological effects (e.g., transduction of biological signals) similar to the biological effects induced when the DAKAR binding partner binds to cell surface DAKAR. Agonistic antibodies can be used to induce DAKAR-mediated cell stimulatory pathways or intercellular communication. Bispecific antibodies can be identified by screening with two separate assays, or with an assay wherein the bispecific antibody serves as a bridge between the first antigen and the second antigen (the latter is coupled to a detectable moiety). Bispecific antibodies that bind DAKAR polypeptides of the invention via a first antigen binding domain will be useful in diagnostic applications and in treating disorders and diseases of the epithelium and related conditions.

Those antibodies that can block binding of the DAKAR polypeptides of the invention to binding partners for DAKAR can be used to inhibit DAKAR-mediated intercellular communication or cell stimulation that results from such binding. Such blocking antibodies can be identified using any suitable assay procedure, such as by testing antibodies for the ability to inhibit binding of DAKAR to certain cells expressing an DAKAR binding partner. Alternatively, blocking antibodies can be identified in assays for the ability to inhibit a biological effect that results from binding of soluble DAKAR to target cells. Antibodies can be assayed for the ability to inhibit DAKAR binding partner-mediated cell stimulatory pathways, for example. Such an antibody can be employed in an in vitro procedure, or administered in vivo to inhibit a biological activity mediated by the entity that generated the antibody. Disorders caused or exacerbated (directly or indirectly) by the interaction of DAKAR with cell surface binding partner receptor thus can be treated. A therapeutic method involves in vivo administration of a blocking antibody to a mammal in an amount effective in inhibiting DAKAR binding partner-mediated biological activity. Monoclonal antibodies are generally preferred for use in such therapeutic methods. In one embodiment, an antigen-binding antibody fragment is employed. Compositions comprising an antibody that is directed against DAKAR, and a physiologically acceptable diluent, excipient, or carrier, are provided herein. Suitable components of such compositions are as described below for compositions containing DAKAR polypeptides.

Also provided herein are conjugates comprising a detectable (e.g., diagnostic) or therapeutic agent, attached to the antibody. Examples of such agents are presented above. The conjugates find use in in vitro or in vivo procedures. The antibodies of the invention can also be used in assays to detect the presence of the polypeptides and/or fragments of the invention, either in vitro or in vivo. The antibodies also can be employed in purifying polypeptides and/or fragments of the invention by immunoaffinity chromatography.

Screening Assays for and Rational Design of Active Compounds that Interact with DAKAR and/or Modulate DAKAR-Associated Events

In one aspect of the present invention a screening method is provided to identify active compounds that modulate DAKAR-associated cell signaling pathways and subsequent downstream events, including cellular events such as apoptosis, differentiation, proliferation, cell cycle and/or senescence. An “active compound” as used herein, comprises all DAKAR polypeptides, polynucleotides, fragments, variants, muteins, fusion proteins, antagonists, agonists, antibodies, and binding partners etc. of the invention that modulate apoptosis, differentiation, proliferation, cell cycle and/or senescence in cells by either inhibiting or enhancing those pathways directly or indirectly through one or more DAKAR-associated events. The method includes contacting putative antagonists and/or agonists of apoptosis, differentiation, proliferation, cell cycle and/or senescence with cells expressing DAKAR, wherein apoptosis, differentiation, proliferation, cell cycle and/or senescence is inhibited or enhanced. The method includes assessing the ability of the active compounds to regulate apoptosis, differentiation, proliferation, cell cycle and/or senescence in cells. Alternatively, a similar method is provided to screen for antagonists and/or agonists of apoptosis, differentiation, proliferation, cell cycle and/or senescence on cells derived from DAKAR deficient mice to assess the efficacy of active compounds on those cellular pathways.

In one embodiment, an assay to screen for agonists and antagonists of DAKAR-induced apoptosis may comprise for example (a) culturing wild-type and DAKAR^{-/-} cells in the presence or absence of an active compound that may enhance or inhibit apoptosis; (b) exposing the wild-type and DAKAR^{-/-} cultures to one or more apoptotic stimuli, such as, but not limited to UV irradiation, ATP and protein synthesis inhibitors and TNF family ligands (Fas, TRAIL, TNF-α, TNF-β, CD30 ligand, CD27 ligand, CD40 ligand, OX-40 ligand, 4-1BB ligand, and the like); (c) assessing the relative difference in measurable apoptosis-associated parameters between the wild-type and DAKAR^{-/-} cultures, such as determining the progressive contraction of cell volume with the preservation of cytoplasmic organelle integrity, condensation of chromatin, the extent of DNA cleavage; and membrane permeability.

In a further embodiment, DAKAR polypeptides, fragments, variants, muteins, fusion proteins, and the like containing the protein kinase domain of DAKAR may be used in an assay of protein kinase activity. Typically this would be carried out by combining DAKAR with radiolabeled ATP (γ³²P-ATP) and a magnesium salt in buffer solution containing a peptide or protein substrate. The peptide substrates are generally from 8-30 amino acids in length and may terminate at the N- or C-terminus with three or more lysine or arginine residues to facilitate binding of the peptide to phosphocellulose paper. The substrate may also be a protein known to be phosphorylated readily by DAKAR. Many such general kinase substrates are known, such as, α or β casein, histone H1, myelin basic protein, etc. After incubation of this reaction mixture at 20-37° C. for a suitable time, the transfer of radioactive phosphate from ATP to the substrate protein or substrate peptide may be monitored, by spotting of the reaction mixture onto phosphocellulose paper, and subsequent washing of the paper with a dilute solution of phosphoric acid, in the case of a peptide substrate, or by application of the reaction products to a gel electrophoresis system followed by autoradiographic detection in the case of proteins. Other methods are available to conveniently measure the DAKAR-mediated transfer of phosphate to substrate proteins, such as the scintillation proximity assay, these methods are well known to those practiced in the art.

In another embodiment, a screening assay comprises (a) culturing wild-type and/or DAKAR^{-/-} cells, cell lysates or subcellular fractions in the presence or absence of an active compound that may enhance or inhibit DAKAR-associated NFκB activation and/or nuclear localization of NFκB, as well as NFκB subunits, such as the p65 subunit; (b) assessing the relative difference in measurable NFκB activation and/or nuclear localization of NFκB parameters between the wild-type and DAKAR^{-/-} cultures. Activation of NFκB may be determined by various techniques well known in the art, such as, but not limited to, gel shift analysis, immunofluorescent staining of the p65 subunit, and the like.

In another embodiment, a screening assay comprises (a) culturing wild-type and/or DAKAR^{-/-} cells, cell lysates or subcellular fractions in the presence or absence of an active compound that may enhance or inhibit directly or indirectly DAKAR-mediated phosphorylation of one or more substrates, as well as association with binding domains such as catalytic domains of other proteins, which may include for example any of the calcium-dependent protein kinases, such as any of the protein kinase C (PKC) isoforms (PKCδ and/or PKCβ, and the like); and (b) assessing the relative difference in measurable DAKAR-mediated association and/or phosphorylation with one or more substrates by conventional techniques, such as substrate-specific probes.

In another embodiment, a screening assay may comprise (a) culturing wild-type and/or DAKAR^{-/-} cells, cell lysates or subcellular fractions in the presence or absence of an active compound that may enhance or inhibit directly or indirectly DAKAR-mediated association with, phosphorylation of and/or degradation of IkB, as well as subunits of the IKK complex including IKKα, IKKβ and IKKγ, and (b) assessing the relative difference in measurable DAKAR-mediated association, phosphorylation and/or degradation of IkB by conventional techniques, such as IkB and IKKα, IKKβ and IKKγ-specific probes.

In yet another embodiment, a screening assay may comprise (a) culturing wild-type and/or DAKAR^{-/-} cells, cell lysates or subcellular fractions in the presence or absence of an active compound that may enhance or inhibit directly or indirectly DAKAR-mediated modulation of transcription and/or translation of differentiation markers, such as, but not limited to filaggrin, profilaggrin, involucrin, keratin markers (such as K1, K2, K2e, K2p, K4, K5, K6, K8, K9, K, K13, K14, K16, K17, K18, K19, and the like); and (b) assessing the relative difference in measurable transcription and/or translation of differentiation markers by conventional techniques, such as differentiation marker-specific probes.

In other aspects of the invention, a screening assay may comprise (a) culturing wild-type and/or DAKAR^{-/-} cells, cell lysates or subcellular fractions in the presence or absence of an active compound that may enhance or inhibit directly or indirectly DAKAR-mediated cellular proliferation; (b) assessing the relative difference in cellular proliferation by (c) measuring the proliferation indices by conventional techniques, such as, but not limited to BrdU and Ki67, as well as other proliferation markers known in the art.

In yet other aspects of the invention, a screening assay may comprise (a) culturing wild-type and/or DAKAR^{-/-} cells, cell lysates or subcellular fractions in the presence or absence of an active compound that may enhance or inhibit directly or indirectly DAKAR-associated caspase activity, such as for example caspase 3 and caspase 8; (b) assessing the relative difference in caspase activity by (c) measuring the enzymatic activity of various DAKAR-associated caspases by conventional techniques, such as, but not limited to measuring DAKAR cleavage products caspase-specific probes.

In various alternative embodiments, screening assays, such as those described above, may be modified to employ a variety of one or more biological and/or cellular “readouts” to determine the influence of DAKAR agonists and/or antagonists on apoptosis, cellular differentiation, proliferation, cell cycle and/or senescence. The following examples comprise cellular readouts, which may be incorporated into any suitable screening assay known in the art, such as cell-based and subcellular-based assays. Further examples may include, assessing the capacity of DAKAR active compounds to modulate regulators of apoptosis and epidermal differentiation, such as members of the Bc1-2 family, including, but not limited to Bc1-2, Bc1-x, bax and bak; mediate intracellular signals in response to Lymphtoxin-α; modulate activity of c-Myc; and to modulate cell cycle regulators, such as cyclin-dependent kinase inhibitors, including for example p16, p21 and p27, as well as positive regulators such as cyclin A, cdk2 and and/or cdc2.

In another embodiment of the present invention, a method for identifying an active compound for the treatment of an individual diagnosed with a DAKAR-associated disorder or a loss of expression of wild-type DAKAR in the individual is provided. One embodiment comprises (a) providing a transgenic knockout mouse whose genome comprises a homozygous disruption in its endogenous DAKAR gene, wherein said homozyogous disruption prevents the expression of a functional DAKAR protein in the cells of the mouse, wherein said homozygous disruption results in said transgenic knockout mouse exhibiting symptoms of keratinocyte aberrations selected from the group consisting of aberrant differentiation, proliferation, cell cycle, senescence, discordant differentiation markers, hypertrophy of the suprabasal layer, diminished or elimination of the stratus corneum, thickened non-wrinkled skin, poorly defined oral and auricular orifices, tail fused to the skin in the area of the rectum and urethra opening, anal, urethra, oral and aral atresia, rigid and stunted limbs, fused digits and a linear red friable streak along the dorsal midline; (b) administering a candidate therapeutic agent, either an agonist or antagonists of DAKAR function, to the transgenic mouse of step (a); and (c) assaying or assessing the therapeutic effects of the candidate therapeutic agent by comparing the symptoms of the keratinocyte disorder in the transgenic mouse which received the candidate agent as in step (b) with the symptoms of the keratinocyte disorder of a transgenic knockout mouse of step (a) which has not received the candidate therapeutic agent, wherein amelioration of one or more of the symptoms of the keratinocyte disorder in the transgenic knockout mouse of step (b) is an indication of the therapeutic effect of the candidate therapeutic agent.

In another aspect, the present invention provides a method of detecting the ability of an active compound to affect the intercellular communication or cell stimulatory activity of a cell. In this aspect, the method comprises: (1) contacting a first group of target cells with a test compound including an DAKAR receptor polypeptide or fragment thereof under conditions appropriate to the particular assay being used; (2) measuring the net rate of intracellular and/or intercellular communication or cell stimulation among the target cells; and (3) observing the net rate of intracellular and/or intercellular communication or cell stimulation among control cells containing the DAKAR receptor polypeptides and/or fragments thereof, in the absence of a test compound, under otherwise identical conditions as the first group of cells. In this embodiment, the net rate of intracellular and/or intercellular communication or cell stimulation in the control cells is compared to that of the cells treated with both the DAKAR molecule as well as a test compound. The comparison will provide a difference in the net rate of intracellular and/or intercellular communication or cell stimulation such that an effector of intracellular and/or intercellular communication or cell stimulation can be identified. The test compound can function as an effector by either activating or up-regulating, or by inhibiting or down-regulating intercellular communication or cell stimulation, and can be detected through this method.

The goal of rational drug design is to produce structural analogs of biologically active polypeptides of interest or of small molecules with which they interact, e.g., inhibitors, agonists, antagonists, etc. Any of these examples can be used to fashion drugs which are more active or stable forms of the polypeptide or which enhance or interfere with the function of a polypeptide in vivo (Hodgson J (1991) Biotechnology 9:19-21). In one approach, the three-dimensional structure of a polypeptide of interest, or of a polypeptide-inhibitor complex, is determined by x-ray crystallography, by nuclear magnetic resonance, or by computer homology modeling or, most typically, by a combination of these approaches. Both the shape and charges of the polypeptide must be ascertained to elucidate the structure and to determine active site(s) of the molecule. Less often, useful information regarding the structure of a polypeptide may be gained by modeling based on the structure of homologous polypeptides. In both cases, relevant structural information is used to design analogous DAKAR-like molecules, to identify efficient inhibitors, or to identify small molecules that bind DAKAR polypeptides. Useful examples of rational drug design include molecules which have improved activity or stability as shown by Braxton S and Wells J A (1992 Biochemistry 31:7796-7801) or which act as inhibitors, agonists, or antagonists of native peptides as shown by Athauda S B et al (1993 J Biochem 113:742-746). The use of DAKAR polypeptide structural information in molecular modeling software systems to assist in inhibitor design and in studying inhibitor-DAKAR polypeptide interaction is also encompassed by the invention. A particular method of the invention comprises analyzing the three dimensional structure of DAKAR polypeptides for likely binding sites of substrates, synthesizing a new molecule that incorporates a predictive reactive site, and assaying the new molecule as described further herein.

It is also possible to isolate a target-specific antibody, selected by functional assay, as described further herein, and then to solve its crystal structure. This approach, in principle, yields a pharmacore upon which subsequent drug design can be based. It is possible to bypass polypeptide crystallography altogether by generating anti-idiotypic antibodies (anti-ids) to a functional, pharmacologically active antibody. As a mirror image of a mirror image, the binding site of the anti-ids would be expected to be an analog of the original antigen. The anti-id could then be used to identify and isolate peptides from banks of chemically or biologically produced peptides. The isolated peptides would then act as the pharmacore.

The purified DAKAR polypeptides of the invention (including polypeptides, polypeptides, fragments, variants, oligomers, and other forms) are useful in a variety of assays. For example, the DAKAR molecules of the present invention can be used to identify binding partners of DAKAR polypeptides, which can also be used to modulate intercellular communication, cell stimulation, or immune cell activity. Alternatively, they can be used to identify non-binding-partner molecules or substances that modulate intercellular communication, cell stimulatory pathways, or immune cell activity.

Assays to Identify Binding Partners. Polypeptides of the DAKAR family and fragments thereof can be used to identify binding partners. For example, they can be tested for the ability to bind a candidate binding partner in any suitable assay, such as a conventional binding assay, as well as a yeast two hybrid system. To illustrate, the DAKAR polypeptide can be labeled with a detectable reagent (e.g., a radionuclide, chromophore, enzyme that catalyzes a calorimetric or fluorometric reaction, and the like). The labeled polypeptide is contacted with cells expressing the candidate binding partner. The cells then are washed to remove unbound labeled polypeptide, and the presence of cell-bound label is determined by a suitable technique, chosen according to the nature of the label.

One example of a binding assay procedure is as follows. A recombinant expression vector containing the candidate binding partner cDNA is constructed. CV1-EBNA-1 cells in 10 cm² dishes are transfected with this recombinant expression vector. CV-1/EBNA-1 cells (ATCC CRL 10478) constitutively express EBV nuclear antigen-1 driven from the CMV Immediate-early enhancer/promoter. CV1-EBNA-1 was derived from the African Green Monkey kidney cell line CV-1 (ATCC CCL 70), as described by McMahan et al. (EMBO J 10:2821, 1991). The transfected cells are cultured for 24 hours, and the cells in each dish then are split into a 24-well plate. After culturing an additional 48 hours, the transfected cells (about 4×10⁴ cells/well) are washed with BM-NFDM, which is binding medium (RPMI 1640 containing 25 mg/ml bovine serum albumin, 2 mg/ml sodium azide, 20 mM Hepes pH 7.2) to which 50 mg/ml nonfat dry milk has been added. The cells then are incubated for 1 hour at 37° C. with various concentrations of, for example, a soluble polypeptide/Fc fusion polypeptide made as set forth above. Cells then are washed and incubated with a constant saturating concentration of a ¹²⁵I-mouse anti-human IgG in binding medium, with gentle agitation for 1 hour at 37° C. After extensive washing, cells are released via trypsinization. The mouse anti-human IgG employed above is directed against the Fc region of human IgG and can be obtained from Jackson Immunoresearch Laboratories, Inc., West Grove, Pa. The antibody is radioiodinated using the standard chloramine-T method. The antibody will bind to the Fc portion of any polypeptide/Fc polypeptide that has bound to the cells. In all assays, non-specific binding of ¹²⁵I-antibody is assayed in the absence of the Fc fusion polypeptide/Fc, as well as in the presence of the Fc fusion polypeptide and a 200-fold molar excess of unlabeled mouse anti-human IgG antibody. Cell-bound ¹²⁵I-antibody is quantified on a Packard Autogamma counter. Affinity calculations (Scatchard, Ann. N.Y. Acad. Sci. 51:660, 1949) are generated on RS/1 (BBN Software, Boston, Mass.) run on a Microvax computer. Binding can also be detected using methods that are well suited for high-throughput screening procedures, such as scintillation proximity assays (Udenfriend et al., 1985, Proc Natl Acad Sci USA 82:8672-8676), homogeneous time-resolved fluorescence methods (Park et al., 1999, Anal Biochem 269:94-104), fluorescence resonance energy transfer (FRET) methods (Clegg R M, 1995, Curr Opin Biotechnol 6:103-110), or methods that measure any changes in surface plasmon resonance when a bound polypeptide is exposed to a potential binding partner, using for example a biosensor such as that supplied by Biacore AB (Uppsala, Sweden). Compounds that can be assayed for binding to DAKAR polypeptides include but are not limited to small organic molecules, such as those that are commercially available—often as part of large combinatorial chemistry compound ‘libraries’—from companies such as Sigma-Aldrich (St. Louis, Mo.), Arqule (Woburn, Mass.), Enzymed (Iowa City, Iowa), Maybridge Chemical Co.(Trevillett, Cornwall, UK), MDS Panlabs (Bothell, Wash.), Pharmacopeia (Princeton, N.J.), and Trega (San Diego, Calif.). Preferred small organic molecules for screening using these assays are usually less than 10K molecular weight and can possess a number of physicochemical and pharmacological properties which enhance cell penetration, resist degradation, and/or prolong their physiological half-lives (Gibbs, J., 1994, Pharmaceutical Research in Molecular Oncology, Cell 79(2):193-198). Compounds including natural products, inorganic chemicals, and biologically active materials such as proteins and toxins can also be assayed using these methods for the ability to bind to DAKAR polypeptides.

Yeast Two-Hybrid or “Interaction Trap” Assays. Where the DAKAR polypeptide binds or potentially binds to another polypeptide (such as, for example, in a kinase/substrate interaction), the nucleic acid encoding the DAKAR polypeptide can also be used in interaction trap assays (such as, for example, that described in Gyuris et al., Cell 75:791-803 (1993)) to identify nucleic acids encoding the other polypeptide with which binding occurs or to identify inhibitors of the binding interaction. Polypeptides involved in these binding interactions can also be used to screen for peptide or small molecule inhibitors or agonists of the binding interaction.

Competitive Binding Assays. Another type of suitable binding assay is a competitive binding assay. To illustrate, biological activity of a variant can be determined by assaying for the variant's ability to compete with the native polypeptide for binding to the candidate binding partner. Competitive binding assays can be performed by conventional methodology. Reagents that can be employed in competitive binding assays include radiolabeled DAKAR and intact cells expressing DAKAR (endogenous or recombinant) on the cell surface. For example, a radiolabeled soluble DAKAR fragment can be used to compete with a soluble DAKAR variant for binding to cell surface receptors. Instead of intact cells, one could substitute a soluble binding partner/Fc fusion polypeptide bound to a solid phase through the interaction of Polypeptide A or Polypeptide G (on the solid phase) with the Fc moiety. Chromatography columns that contain Polypeptide A and Polypeptide G include those available from Pharmacia Biotech, Inc., Piscataway, N.J.

Assays to Identify Modulators of Intercellular Communication, Cell Stimulation, or Immune Cell Activity. The influence of DAKAR on intercellular communication, cell stimulation, or immune cell activity can be manipulated to control these activities in target cells. For example, the disclosed DAKAR polypeptides, nucleic acids encoding the disclosed DAKAR polypeptides, or agonists or antagonists of such polypeptides can be administered to a cell or group of cells to induce, enhance, suppress, or arrest cellular communication, cell stimulation, or activity in the target cells. Identification of DAKAR polypeptides, agonists or antagonists that can be used in this manner can be carried out via a variety of assays known to those skilled in the art. Included in such assays are those that evaluate the ability of an DAKAR polypeptide to influence intercellular communication, cell stimulation or activity. Such an assay would involve, for example, the analysis of immune cell interaction in the presence of an DAKAR polypeptide. In such an assay, one would determine a rate of communication or cell stimulation in the presence of the DAKAR polypeptide and then determine if such communication or cell stimulation is altered in the presence of a candidate agonist or antagonist or another DAKAR polypeptide. Exemplary assays for this aspect of the invention include cytokine secretion assays, T-cell co-stimulation assays, and mixed lymphocyte reactions involving antigen presenting cells and T cells. These assays are well known to those skilled in the art.

Cell Proliferation, Cell Death, Cell Differentiation, and Cell Adhesion Assays. A polypeptide of the present invention may exhibit cytokine, cell proliferation (either inducing or inhibiting), or cell differentiation (either inducing or inhibiting) activity, or may induce production of other cytokines in certain cell populations. Many polypeptide factors discovered to date have exhibited such activity in one or more factor-dependent cell proliferation assays, and hence the assays serve as a convenient confirmation of cell stimulatory activity. The activity of agonists and/or antagonists of DAKAR of the present invention is evidenced by any one of a number of routine factor-dependent cell proliferation assays for cell lines including, without limitation, 32D, DA2, DA1G, T10, B9, B9/11, BaF3, MC9/G, M+(preB M+), 2E8, RB5, DA1, 123, T1165, HT2, CTLL2, TF-1, Mo7e and CMK. The activity of a DAKAR polypeptide of the invention may, among other means, be measured by the following methods:

Assays for T-cell or thymocyte proliferation include without limitation those described in: Current Protocols in Immunology, Coligan et al. eds, Greene Publishing Associates and Wiley-Interscience (pp. 3.1-3.19: In vitro assays for mouse lymphocyte function; Chapter 7: Immunologic studies in humans); Takai et al., J. Immunol. 137:3494-3500, 1986; Bertagnolli et al., J. Immunol. 145:1706-1712, 1990; Bertagnolli et al., Cellular Immunology 133:327-341, 1991; Bertagnolli et al., J. Immunol. 149:3778-3783, 1992; Bowman et al., J. Immunol. 152:1756-1761, 1994.

Assays for cytokine production and/or proliferation of spleen cells, lymph node cells or thymocytes include, without limitation, those described in: Kruisbeek and Shevach, 1994, Polyclonal T cell stimulation, in Current Protocols in Immunology, Coligan et al. eds. Vol 1 pp. 3.12.1-3.12.14, John Wiley and Sons, Toronto; and Schreiber, 1994. Measurement of mouse and human interferon gamma in Current Protocols in Immunology, Coligan et al. eds. Vol 1 pp. 6.8.1-6.8.8, John Wiley and Sons, Toronto.

Assays for proliferation and differentiation of hematopoietic and lymphopoietic cells include, without limitation, those described in: Bottomly et al., 1991, Measurement of human and murine interleukin 2 and interleukin 4, in Current Protocols in Immunology, Coligan et al. eds. Vol 1 pp. 6.3.1-6.3.12, John Wiley and Sons, Toronto; deVries et al., J Exp Med 173:1205-1211, 1991; Moreau et al., Nature 336:690-692, 1988; Greenberger et al., Proc Natl Acad Sci. USA 80:2931-2938, 1983; Nordan, 1991, Measurement of mouse and human interleukin 6, in Current Protocols in Immunology Coligan et al. eds. Vol 1 pp. 6.6.1-6.6.5, John Wiley and Sons, Toronto; Smith et al., Proc Natl Acad Sci USA 83:1857-1861, 1986; Bennett et al., 1991, Measurement of human interleukin 11, in Current Protocols in Immunology Coligan et al. eds. Vol 1 pp. 6.15.1 John Wiley and Sons, Toronto; Ciarletta et al., 1991, Measurement of mouse and human Interleukin 9, in Current Protocols in Immunology Coligan et al. eds. Vol 1 pp. 6.13.1, John Wiley and Sons, Toronto.

Assays for T-cell clone responses to antigens (which will identify, among others, polypeptides that affect APC-T cell interactions as well as direct T-cell effects by measuring proliferation and cytokine production) include, without limitation, those described in: Current Protocols in Immunology, Coligan et al. eds, Greene Publishing Associates and Wiley-Interscience (Chapter 3: In vitro assays for mouse lymphocyte function; Chapter 6: Cytokines and their cellular receptors; Chapter 7: Immunologic studies in humans); Weinberger et al., Proc Natl Acad Sci USA 77:6091-6095, 1980; Weinberger et al., Eur. J. Immun. 11:405-411, 1981; Takai et al., J Immunol. 137:3494-3500, 1986; Takai et al., J. Immunol. 140:508-512, 1988

Assays for thymocyte or splenocyte cytotoxicity include, without limitation, those described in: Current Protocols in Immunology, Coligan et al. eds, Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Bowman et al., J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, polypeptides that modulate T-cell dependent antibody responses and that affect Th1/Th2 profiles) include, without limitation, those described in: Maliszewski, J Immunol 144:3028-3033, 1990; and Mond and Brunswick, 1994, Assays for B cell function: in vitro antibody production, in Current Protocols in Immunology Coligan et al. eds. Vol 1 pp. 3.8.1-3.8.16, John Wiley and Sons, Toronto.

Mixed lymnphocyte reaction (MLR) assays (which will identify, among others, polypeptides that generate predominantly Th1 and CTL responses) include, without limitation, those described in: Current Protocols in Immunology, Coligan et al. eds, Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992.

Dendritic cell-dependent assays (which will identify, among others, polypeptides expressed by dendritic cells that activate naive T-cells) include, without limitation, those described in: Guery et al., J. Immunol 134:536-544, 1995; Inaba et al., J Exp Med 173:549-559, 1991; Macatonia et al., J Immunol 154:5071-5079, 1995; Porgador et al., J Exp Med 182:255-260, 1995; Nair et al., J. Virology 67:4062-4069, 1993; Huang et al., Science 264:961-965, 1994; Macatonia et al., J Exp Med 169:1255-1264, 1989; Bhardwaj et al., J Clin Invest 94:797-807, 1994; and Inaba et al., J Exp Med 172:631-640,1990.

Assays for lymphocyte survival/apoptosis (which will identify, among others, polypeptides that prevent apoptosis after superantigen induction and polypeptides that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, J Immunol 145:4037-4045, 1990; Zamai et al., Cytometry 14:891-897, 1993; Gorczyca et al., International Journal of Oncology 1:639-648, 1992.

Assays for polypeptides that influence early steps of T-cell commitment and development include, without limitation, those described in: Antica et al., Blood 84:111-117, 1994; Fine et al., Cell Immunol 155:111-122, 1994; Galy et al., Blood 85:2770-2778, 1995; Toki et al., Proc Natl Acad Sci. USA 88:7548-7551, 1991

Assays for embryonic stem cell differentiation (which will identify, among others, polypeptides that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for stem cell survival and differentiation (which will identify, among others, polypeptides that regulate lympho-hematopoiesis) include, without limitation, those described in: Methylcellulose colony forming assays, Freshney, 1994, In Culture of Hematopoietic Cells, Freshney et al. eds. pp. 265-268, Wiley-Liss, Inc., New York, N.Y.; Hirayama et al., Proc. Natl. Acad. Sci. USA 89:5907-5911, 1992; Primitive hematopoietic colony forming cells with high proliferative potential, McNiece and Briddell, 1994, In Culture of Hematopoietic Cells, Freshney et al. eds. pp. 23-39, Wiley-Liss, Inc., New York, N.Y.; Neben et al., Experimental Hematology 22:353-359, 1994; Ploemacher, 1994, Cobblestone area forming cell assay, In Culture of Hematopoietic Cells, Freshney et al. eds. pp. 1-21, Wiley-Liss, Inc., New York, N.Y.; Spooncer et al., 1994, Long term bone marrow cultures in the presence of stromal cells, In Culture of Hematopoietic Cells, Freshney et al. eds. pp. 163-179, Wiley-Liss, Inc., New York, N.Y.; Sutherland, 1994, Long term culture initiating cell assay, In Culture of Hematopoietic Cells, Freshney et al. eds. Vol pp. 139-162, Wiley-Liss, Inc., New York, N.Y.

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium). Assays for wound healing activity include, without limitation, those described in: Winter, Epidermal Wound Healing, pps. 71-112 (Maibach and Rovee, eds.), Year Book Medical Publishers, Inc., Chicago, as modified by Eaglestein and Mertz, J. Invest. Dermatol 71:382-84 (1978).

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc. Natl. Acad. Sci. USA 83:3091-3095, 1986.

Assays for cell movement and adhesion include, without limitation, those described in: Current Protocols in Immunology Coligan et al. eds, Greene Publishing Associates and Wiley-Interscience (Chapter 6.12, Measurement of alpha and beta chemokines 6.12.1-6.12.28); Taub et al., J. Clin. Invest. 95:1370-1376, 1995; Lind et al. APMIS 103:140-146, 1995; Muller et al., Eur. J Immunol. 25:1744-1748; Gruber et al., J Immunol. 152:5860-5867, 1994; Johnston et al., J Immunol. 153:1762-1768, 1994

Assay for hemostatic and thrombolytic activity include, without limitation, those described in: Linet et al., J. Clin. Pharmacol. 26:131-140, 1986; Burdick et al., Thrombosis Res. 45:413-419, 1987; Humphrey et al., Fibrinolysis 5:71-79 (1991); Schaub, Prostaglandins 35:467-474, 1988.

Assays for receptor-ligand activity include without limitation those described in: Current Protocols in Immunology Coligan et al. eds, Greene Publishing Associates and Wiley-Interscience (Chapter 7.28, Measurement of cellular adhesion under static conditions 7.28.1-7.28.22), Takai et al., Proc. Natl. Acad. Sci. USA 84:6864-6868, 1987; Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stitt et al., Cell 80:661-670, 1995.

Assays for cadherin adhesive and invasive suppressor activity include, without limitation, those described in: Hortsch et al., J Biol Chem 270(32):18809-18817, 1995; Miyaki et al., Oncogene 11:2547-2552, 1995; Ozawa et al., Cell 63:1033-1038, 1990.

Diagnostic and Other Uses of DAKAR Polypeptides and Nucleic Acids

The nucleic acids encoding the DAKAR polypeptides provided by the present invention can be used for numerous diagnostic or other useful purposes. The nucleic acids of the invention can be used to express recombinant polypeptide for analysis, characterization or therapeutic use; as markers for tissues in which the corresponding polypeptide is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel nucleic acids; for selecting and making oligomers for attachment to a “gene chip” or other support, including for examination of expression patterns; to raise anti-polypeptide antibodies using DNA immunization techniques; as an antigen to raise anti-DNA antibodies or elicit another immune response, and. for gene therapy. Uses of DAKAR polypeptides and fragmented polypeptides include, but are not limited to, the following: purifying polypeptides and measuring the activity thereof; delivery agents; therapeutic and research reagents; molecular weight and isoelectric focusing markers; controls for peptide fragmentation; identification of unknown polypeptides; and preparation of antibodies. Any or all nucleic acids suitable for these uses are capable of being developed into reagent grade or kit format for commercialization as products. Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

Probes and Primers. Among the uses of the disclosed DAKAR nucleic acids, and combinations of fragments thereof, is the use of fragments as probes or primers. Such fragments generally comprise at least about 17 contiguous nucleotides of a DNA sequence. In other embodiments, a DNA fragment comprises at least 30, or at least 60, contiguous nucleotides of a DNA sequence. The basic parameters affecting the choice of hybridization conditions and guidance for devising suitable conditions are set forth by Sambrook et al., 1989 and are described in detail above. Using knowledge of the genetic code in combination with the amino acid sequences set forth above, sets of degenerate oligonucleotides can be prepared. Such oligonucleotides are useful as primers, e.g., in polymerase chain reactions (PCR), whereby DNA fragments are isolated and amplified. In certain embodiments, degenerate primers can be used as probes for non-human genetic libraries. Such libraries would include but are not limited to cDNA libraries, genomic libraries, and even electronic EST (express sequence tag) or DNA libraries. Homologous sequences identified by this method would then be used as probes to identify non-human DAKAR homologues.

Chromosome Mapping. The nucleic acids encoding DAKAR polypeptides, and the disclosed fragments and combinations of these nucleic acids, can be used by those skilled in the art using well-known techniques to identify the human chromosome to which these nucleic acids map. Useful techniques include, but are not limited to, using the sequence or portions, including oligonucleotides, as a probe in various well-known techniques such as radiation hybrid mapping (high resolution), in situ hybridization to chromosome spreads (moderate resolution), and Southern blot hybridization to hybrid cell lines containing individual human chromosomes (low resolution). For example, chromosomes can be mapped by radiation hybridization. PCR is performed using the Whitehead Institute/MIT Center for Genome Research Genebridge4 panel of 93 radiation hybrids, using primers that lie within a putative exon of the gene of interest and which amplify a product from human genomic DNA, but do not amplify hamster genomic DNA. The PCR results are converted into a data vector that is submitted to the Whitehead/MIT Radiation Mapping site (www-seq.wi.mit.edu). The data is scored and the chromosomal assignment and placement relative to known Sequence Tag Site (STS) markers on the radiation hybrid map is provided. Alternatively, the genomic sequences corresponding to nucleic acids encoding a DAKAR polypeptide are mapped by comparison to sequences in public and proprietary databases, such as the GenBank non-redundant database (www.ncbi.nlm.nih.gov/BLAST), Locuslink (www.ncbi.nlm.nih.gov:80/LocusLink/), Unigene (www.ncbi.nlm.nih.gov/cgi-bin/UniGene), AceView (www.ncbi.nlm.nih.gov/AceView), Online Mendelian Inheritance in Man (OMIM) (www.ncbi.nlm.nih.gov/Omim), Gene Map Viewer (www.ncbi.nlm.nih.gov/genemap), and proprietary databases such as the Celera Discovery System (www.celera.com). These computer analyses of available genomic sequence information can provide the identification of the specific chromosomal location of human genomic sequences corresponding to sequences encoding human DAKAR polypeptides, and the unique genetic mapping relationships between the DAKAR genomic sequences and the genetic map locations of known human genetic disorders.

Diagnostics and Gene Therapy. The nucleic acids encoding DAKAR polypeptides, and the disclosed fragments and combinations of these nucleic acids can be used by one skilled in the art using well-known techniques to analyze abnormalities associated with the genes corresponding to these polypeptides. This enables one to distinguish conditions in which this marker is rearranged or deleted. In addition, nucleic acids of the invention or a fragment thereof can be used as a positional marker to map other genes of unknown location. The DNA can be used in developing treatments for any disorder mediated (directly or indirectly) by defective, or insufficient amounts of, the genes corresponding to the nucleic acids of the invention. Disclosure herein of native nucleotide sequences permits the detection of defective genes, and the replacement thereof with normal genes. Defective genes can be detected in in vitro diagnostic assays, and by comparison of a native nucleotide sequence disclosed herein with that of a gene derived from a person suspected of harboring a defect in this gene.

Methods of Screening for Binding Partners. The DAKAR polypeptides of the invention each can be used as reagents in methods to screen for or identify binding partners. For example, the DAKAR polypeptides can be attached to a solid support material and may bind to their binding partners in a manner similar to affinity chromatography. In particular embodiments, a polypeptide is attached to a solid support by conventional procedures. As one example, chromatography columns containing functional groups that will react with functional groups on amino acid side chains of polypeptides are available (Pharmacia Biotech, Inc., Piscataway, N.J.). In an alternative, a polypeptide/Fc polypeptide (as discussed above) is attached to protein A- or protein G-containing chromatography columns through interaction with the Fc moiety. The DAKAR polypeptides also find use in identifying cells that express a DAKAR binding partner. Purified DAKAR polypeptides are bound to a solid phase such as a column chromatography matrix or a similar suitable substrate. For example, magnetic microspheres can be coated with the polypeptides and held in an incubation vessel through a magnetic field. Suspensions of cell mixtures or cell lysates of isolated cells containing potential binding-partner-expressing cells are contacted with the solid phase having the polypeptides thereon. Cells expressing the binding partner on the cell surface bind to the fixed polypeptides, and unbound cells are washed away. In an alternative format, intracellular binding partners or substrates DAKAR from cell lysates bind to DAKAR polypeptides and unbound proteins are removed. Alternatively, DAKAR polypeptides can be conjugated to a detectable moiety, then incubated with cells to be tested for binding partner expression. After incubation, unbound labeled matter is removed and the presence or absence of the detectable moiety on the cells is determined. In a further alternative, mixtures of cells or cell lysates suspected of expressing the binding partner are incubated with biotinylated polypeptides. Incubation periods are typically at least one hour in duration to ensure sufficient binding. The resulting mixture then is passed through a column packed with avidin-coated beads, whereby the high affinity of biotin for avidin provides binding of the desired cells or binding partners to the beads. Procedures for using avidin-coated beads are known (see Berenson, et al. J. Cell. Biochem., 10D:239, 1986). Washing to remove unbound material, and the release of the bound cells or binding partners, are performed using conventional methods. In some instances, the above methods for screening for or identifying binding partners may also be used or modified to isolate or purify such binding partner molecules or cells expressing them.

Measuring Biological Activity. Polypeptides also find use in measuring the biological activity of DAKAR-binding polypeptides in terms of their binding affinity. The polypeptides thus can be employed by those conducting “quality assurance” studies, e.g., to monitor shelf life and stability of polypeptide under different conditions. For example, the polypeptides can be employed in a binding affinity study to measure the biological activity of a binding partner polypeptide that has been stored at different temperatures, or produced in different cell types. The polypeptides also can be used to determine whether biological activity is retained after modification of a binding partner polypeptide (e.g., chemical modification, truncation, mutation, etc.). The binding affinity of the modified polypeptide is compared to that of an unmodified binding polypeptide to detect any adverse impact of the modifications on biological activity of the binding polypeptide. The biological activity of a binding polypeptide thus can be ascertained before it is used in a research study, for example.

Carriers and Delivery Agents. The polypeptides also find use as carriers for delivering agents attached thereto to cells bearing identified binding partners. The polypeptides thus can be used to deliver diagnostic or therapeutic agents to such cells (or to other cell types found to express binding partners on the cell surface) in in vitro or in vivo procedures. Detectable (diagnostic) and therapeutic agents that can be attached to a polypeptide include, but are not limited to, toxins, other cytotoxic agents, drugs, radionuclides, chromophores, enzymes that catalyze a colorimetric or fluorometric reaction, and the like, with the particular agent being chosen according to the intended application. Among the toxins are ricin, abrin, diphtheria toxin, Pseudomonas aeruginosa exotoxin A, ribosomal inactivating polypeptides, mycotoxins such as trichothecenes, and derivatives and fragments (e.g., single chains) thereof. Radionuclides suitable for diagnostic use include, but are not limited to, ¹²³I, ¹³¹I, ^{99m}Tc, ¹¹¹In, and ⁷⁶Br. Examples of radionuclides suitable for therapeutic use are 131I, ²¹¹At, ⁷⁷Br, ¹⁸⁶Re, ¹⁸⁸Re, ²¹²Pb, ²¹²Bi, ¹⁰⁹Pd, ⁶⁴Cu, and ⁶⁷Cu. Such agents can be attached to the polypeptide by any suitable conventional procedure. The polypeptide comprises functional groups on amino acid side chains that can be reacted with functional groups on a desired agent to form covalent bonds, for example. Alternatively, the polypeptide or agent can be derivatized to generate or attach a desired reactive functional group. The derivatization can involve attachment of one of the bifunctional coupling reagents available for attaching various molecules to polypeptides (Pierce Chemical Company, Rockford, Ill.). A number of techniques for radiolabeling polypeptides are known. Radionuclide metals can be attached to polypeptides by using a suitable bifunctional chelating agent, for example. Conjugates comprising polypeptides and a suitable diagnostic or therapeutic agent (preferably covalently linked) are thus prepared. The conjugates are administered or otherwise employed in an amount appropriate for the particular application.

Therapeutic Applications

DAKAR polypeptides, fragments, variants, antagonists, agonists, antibodies, and binding partners of the invention, as well as polynucleotides encoding the aforementioned, are likely to be useful for treating a number of medical conditions and diseases as described further herein. The therapeutic molecule or molecules to be used will depend on the etiology of the condition to be treated and the biological pathways involved. Agonists, antagonists, variants, muteins, fragments, and binding partners of DAKAR polypeptides may have effects similar to or different from DAKAR polypeptides. Therefore, in the following paragraphs “DAKAR polypeptides, agonists and/or antagonists” refers to all DAKAR polypeptides, polynucleotides, fragments, variants, muteins, fusion proteins, antagonists, agonists, antibodies, and binding partners etc. of the invention, and it is understood that a specific molecule or molecules can be selected from those provided as embodiments of the invention by individuals of skill in the art, according to the biological and therapeutic considerations described herein.

The disclosed DAKAR polypeptides, agonists and/or antagonists, compositions and combination therapies described herein are useful in medicines and methods of treatment involving disorders of the epithelium. Such disorders include differentiative and proliferative disorders of the epithelium; hyperplastic growth of epithelium; acantholytic diseases, including Darier's disease, keratosis follicularis and pemphigus vulgaris; paraneoplastic pemphigus; aphthous stomatitis; bullous pemphigoid; epidermolysis bullosa, including bullous congenital icthyosiform erythroderma and Dowling-Meara type; pachyonychia congenita; hyperkeratosis, including epidermolytic hyperkeratosis; icthyosis, including icthyosis bullosa of Siemens and icthyosis vulgaris; palmoplantar keratoderma, including epidermolytic and non-epidermolytic palmoplantar keratoderma; pachyonychia congenita, including Jadassohn-Lewandowsky type; white sponge nevus; tricho-dento-osseous syndrome; tooth agenesis; autosomal dominant craniosyntosis, including Boston type; Papillon-Lefevre syndrome; Haim-Munk syndrome; prebubertal periodontis; bums; eczema; erythema, including erythema multiforme and erythema multiforme bullosum (Stevens-Johnson syndrome); inflammatory skin disease, including psoriasis, leukocutoclastic vasculitis, allergic contact dermatitis, pemphigus vulgaris, erythema multifome; lupus erythematosus; lichen planus; linear IgA bullous disease (chronic bullous dermatosis of childhood); loss of skin elasticity; fragility of the epidermis; ulcerations, including chronic ulcerations, diabetes-associated ulcerations and mucosal surface ulcers; neutrophilic dermatitis (Sweet's syndrome); pityriasis rubra pilaris; psoriasis; pyoderma gangrenosum; acne; acne rosacea; alopecia areata; and toxic epidermal necrolysis.

In other aspects of the present invention, DAKAR polypeptides, polynucleotides, fragments, variants, muteins, fusion proteins, antagonists, agonists, antibodies, and binding partners etc. may be used to enhance or inhibit apoptosis; DAKAR-associated NFκB activation and/or nuclear localization of NFκB, as well as NFκB subunits, such as the p65 subunit; DAKAR-mediated phosphorylation of one or more substrates, as well as association with binding domains such as catalytic domains of other proteins, which may include for example any of the calcium-dependent protein kinases, such as any of the protein kinase C (PKC) isoforms (PKCδ and/or PKCβ, and the like); DAKAR-mediated association with, phosphorylation of and/or degradation of IkB, as well as subunits of the IKK complex including IKKα, IKKβ and IKKγ, DAKAR-mediated modulation of transcription and/or translation of differentiation markers, such as, but not limited to filaggrin, profilaggrin, involucrin, keratin markers (such as K1, K2, K2e, K2p, K4, K5, K6, K8, K9, K10, K13, K14, K16, K17, K18, K19, and the like); DAKAR-mediated cellular proliferation; DAKAR-associated caspase activity, modulate regulators of apoptosis and epidermal differentiation, such as members of the Bc1-2 family, including, but not limited to Bc1-2, Bc1-x, bax and bak; mediate intracellular signals in response to Lymphtoxin-α; modulate activity of c-Myc; and to modulate cell cycle regulators, such as cyclin-dependent kinase inhibitors, including for example p16, p21 and p27, as well as positive regulators such as cyclin A, cdk2 and/or cdc2; and thereby alleviate symptoms of diseases and disorders related thereto.

The disclosed DAKAR polypeptides agonists and/or antagonists, compositions and combination therapies described herein are useful in medicines for treating bacterial, viral or protozoal infections, and complications resulting therefrom. One such disease is Mycoplasma pneumonia. In addition, provided herein is the use of DAKAR polypeptides and/or antagonists to treat AIDS and related conditions, such as AIDS dementia complex, AIDS associated wasting, lipidistrophy due to antiretroviral therapy; and Kaposi's sarcoma. Provided herein is the use of DAKAR polypeptides agonists and/or antagonists for treating protozoal diseases, including malaria and schistosomiasis. Additionally provided is the use of DAKAR polypeptides agonists and/or antagonists to treat erythema nodosum leprosum; bacterial or viral meningitis; tuberculosis, including pulmonary tuberculosis; and pneumonitis secondary to a bacterial or viral infection. Provided also herein is the use of DAKAR polypeptides and/or antagonists to prepare medicaments for treating louse-borne relapsing fevers, such as that caused by Borrelia recurrentis. The DAKAR polypeptides, polynucleotides, agonists and/or antagonists, compositions and combination therapies of the invention can also be used to prepare a medicament for treating conditions caused by Herpes viruses, such as herpetic stromal keratitis, corneal lesions, and virus-induced corneal disorders. In addition, DAKAR polypeptides, polynucleotides, agonists and/or antagonists, compositions and combination therapies can be used in treating human papillomavirus infections.

Cardiovascular disorders are treatable with the disclosed DAKAR polypeptides, polynucleotides, agonists and/or antagonists, pharmaceutical compositions or combination therapies, including aortic aneurisms; arteritis; vascular occlusion, including cerebral artery occlusion; complications of coronary by-pass surgery; ischemia/reperfusion injury; heart disease, including atherosclerotic heart disease, myocarditis, including chronic autoimmune myocarditis and viral myocarditis; heart failure, including chronic heart failure (CHF), cachexia of heart failure; myocardial infarction; restenosis after heart surgery; silent myocardial ischemia; post-implantation complications of left ventricular assist devices; Raynaud's phenomena; thrombophlebitis; vasculitis, including Kawasaki's vasculitis; giant cell arteritis, Wegener's granulomatosis; and Schoenlein-Henoch purpura.

A combination of at least one DAKAR polypeptides, polynucleotides, agonists and/or antagonist and one or more other anti-angiogenesis factors may be used to treat solid tumors, thereby reducing the vascularization that nourishes the tumor tissue. Suitable anti-angiogenic factors for such combination therapies include IL-8 inhibitors, angiostatin, endostatin, kringle 5, inhibitors of vascular endothelial growth factor (such as antibodies against vascular endothelial growth factor), angiopoietin-2 or other antagonists of angiopoietin-1, antagonists of platelet-activating factor and antagonists of basic fibroblast growth factor

In addition, the subject DAKAR polypeptides, polynucleotides, agonists and/or antagonist, compositions and combination therapies are used to treat chronic pain conditions, such as chronic pelvic pain, including chronic prostatitis/pelvic pain syndrome. As a further example, DAKAR polypeptides, polynucleotides, agonists and/or antagonists, compositions and combination therapies of the invention are used to treat post-herpetic pain.

Provided also are methods for using DAKAR polypeptides, polynucleotides, agonists and/or antagonists, compositions and combination therapies to treat various disorders of the endocrine system. For example, the DAKAR polypeptides, polynucleotides, agonists and/or antagonists, compositions and combination therapies are used to treat juvenile onset diabetes (includes autoimmune and insulin-dependent types of diabetes) and also to treat maturity onset diabetes (includes non-insulin dependent and obesity-mediated diabetes). In addition, the subject compounds, compositions and combination therapies are used to treat secondary conditions associated with diabetes, such as diabetic retinopathy, kidney transplant rejection in diabetic patients, obesity-mediated insulin resistance, and renal failure, which itself may be associated with proteinurea and hypertension. Other endocrine disorders also are treatable with these compounds, compositions or combination therapies, including polycystic ovarian disease, X-linked adrenoleukodystrophy, hypothyroidism and thyroiditis, including Hashimoto's thyroiditis (i.e., autoimmune thyroiditis).

Conditions of the gastrointestinal system also are treatable with DAKAR polypeptides, polynucleotides, agonists and/or antagonists, compositions and combination therapies, including coeliac disease; Crohn's disease; ulcerative colitis; idiopathic gastroparesis; pancreatitis, including chronic pancreatitis and lung injury associated with acute pancreatitis; and ulcers, including gastric and duodenal ulcers.

Included also are methods for using the subject DAKAR polypeptides, polynucleotides, agonists and/or antagonists, compositions and combination therapies for treating disorders of the genitourinary system, such as glomerulonephritis, including autoimmune glomerulonephritis, glomerulonephritis due to exposure to toxins or glomerulonephritis secondary to infections with haemolytic streptococci or other infectious agents. Also treatable with the compounds, compositions and combination therapies of the invention are uremic syndrome and its clinical complications (for example, renal failure, anemia, and hypertrophic cardiomyopathy), including uremic syndrome associated with exposure to environmental toxins, drugs or other causes. Further conditions treatable with the compounds, compositions and combination therapies of the invention are complications of hemodialysis; prostate conditions, including benign prostatic hypertrophy, nonbacterial prostatitis and chronic prostatitis; and complications of hemodialysis.

Also provided herein are methods for using DAKAR polypeptides, polynucleotides, agonists and/or antagonists, compositions and combination therapies to treat various hematologic and oncologic disorders. For example, DAKAR polypeptides and/or antagonists are used to treat various forms of cancer, including acute myelogenous leukemia, Epstein-Barr virus-positive nasopharyngeal carcinoma, glioma, colon, stomach, prostate, renal cell, cervical and ovarian cancers, lung cancer (SCLC and NSCLC), including cancer-associated cachexia, fatigue, asthenia, paraneoplastic syndrome of cachexia and hypercalcemia.

Additional diseases treatable with the subject DAKAR polypeptides and/or antagonists, compositions and/or combination therapies are solid tumors, including sarcoma, osteosarcoma, and carcinoma, such as adenocarcinoma (for example, breast cancer); melanotic neoplasia, including melanocytic nevus, radial and vertical growth phase melanoma; squamous cell neoplasia, including seborrheic keratosis, actinic keratosis, basal cell carcinomas and squamous cell carcinoma. In addition, the subject compounds, compositions and/or combination therapies are useful for treating leukemia, including acute myelogenous leukemia, chronic or acute lymphoblastic leukemia and hairy cell leukemia. Other malignancies with invasive metastatic potential can be treated with the subject compounds, compositions and combination therapies, including multiple myeloma. In addition, the disclosed DAKAR polypeptides and/or antagonists, compositions and combination therapies can be used to treat anemias and hematologic disorders, including anemia of chronic disease, aplastic anemia, including Fanconi's aplastic anemia; idiopathic thrombocytopenic purpura (ITP); myelodysplastic syndromes (including refractory anemia, refractory anemia with ringed sideroblasts, refractory anemia with excess blasts, refractory anemia with excess blasts in transformation); myelofibrosis/myeloid metaplasia; and sickle cell vasocclusive crisis.

Various lymphoproliferative disorders also are treatable with the disclosed DAKAR polypeptides and/or antagonists, compositions or combination therapies. These include, but are not limited to autoimmune lymphoproliferative syndrome (ALPS), chronic lymphoblastic leukemia, hairy cell leukemia, chronic lymphatic leukemia, peripheral T-cell lymphoma, small lymphocytic lymphoma, mantle cell lymphoma, follicular lymphoma, Burkitt's lymphoma, Epstein-Barr virus-positive T cell lymphoma, histiocytic lymphoma, Hodgkin's disease, diffuse aggressive lymphoma, acute lymphatic leukemias, T gamma lymphoproliferative disease, cutaneous B cell lymphoma, cutaneous T cell lymphoma (i.e., mycosis fungoides) and Sézary syndrome.

In addition, the subject DAKAR polypeptides and/or antagonists, compositions and combination therapies are used to treat hereditary conditions such as Gaucher's disease, Huntington's disease, linear IgA disease, and muscular dystrophy.

Other conditions treatable by the disclosed DAKAR polypeptides and/or antagonists, compositions and combination therapies include those resulting from injuries to the head or spinal cord, and including subdural hematoma due to trauma to the head.

The disclosed DAKAR polypeptides and/or antagonists, compositions and combination therapies are further used to treat conditions of the liver such as hepatitis, including acute alcoholic hepatitis, acute drug-induced or viral hepatitis, hepatitis A, B and C, sclerosing cholangitis and inflammation of the liver due to unknown causes.

In addition, the disclosed DAKAR polypeptides and/or antagonists, compositions and combination therapies are used to treat various disorders that involve hearing loss and that are associated with abnormal DAKAR expression. One of these is inner ear or cochlear nerve-associated hearing loss that is thought to result from an autoimmune process, i.e., autoimmune hearing loss. This condition currently is treated with steroids, methotrexate and/or cyclophosphamide, which may be administered concurrently with the DAKAR polypeptides and/or antagonists. Also treatable with the disclosed DAKAR polypeptides and/or antagonists, compositions, and combination therapies is cholesteatoma, a middle ear disorder often associated with hearing loss.

In addition, the subject invention provides DAKAR polypeptides agonists and/or antagonists, compositions and combination therapies for the treatment of non-arthritic medical conditions of the bones and joints. This encompasses osteoclast disorders that lead to bone loss, such as but not limited to osteoporosis, including post-menopausal osteoporosis, periodontitis resulting in tooth loosening or loss, and prosthesis loosening after joint replacement (generally associated with an inflammatory response to wear debris). This latter condition also is called “orthopedic implant osteolysis.” Another condition treatable by administering DAKAR polypeptides and/or antagonists, is temporal mandibular joint dysfunction (TMJ).

A number of pulmonary disorders also can be treated with the disclosed DAKAR polypeptides and/or antagonists, compositions and combination therapies. The disclosed compounds, compositions and combination therapies of the invention also are useful for treating broncho-pulmonary dysplasia (BPD); lymphangioleiomyomatosis; and chronic fibrotic lung disease of pre-term infants. In addition, the compounds, compositions and combination therapies of the invention are used to treat occupational lung diseases, including asbestosis, coal worker's pneumoconiosis, silicosis or similar conditions associated with long-term exposure to fine particles. In other aspects of the invention, the disclosed compounds, compositions and combination therapies are used to treat pulmonary disorders, including chronic obstructive pulmonary disease (COPD) associated with chronic bronchitis and/or emphysema; fibrotic lung diseases, such as cystic fibrosis, idiopathic pulmonary fibrosis and radiation-induced pulmonary fibrosis; pulmonary sarcoidosis; and allergies, including allergic rhinitis, contact dermatitis, atopic dermatitis and asthma.

Cystic fibrosis is an inherited condition characterized primarily by the accumulation of thick mucus, predisposing the patient to chronic lung infections and obstruction of the pancreas, which results in malabsorption of nutrients and malnutrition. DAKAR polypeptides and/or antagonists may be administered to treat cystic fibrosis. If desired, treatment with DAKAR polypeptides and/or antagonists may be administered concurrently with corticosteroids, mucus-thinning agents such as inhaled recombinant deoxyribonuclease I (such as PULMOZYME®; Genentech, Inc.) and/or inhaled tobramycin (TOBI®; Pathogenesis, Inc.). The DAKAR polypeptides and/or antagonists of the invention also may be administered concurrently with corrective gene therapy, drugs that stimulate cystic fibrosis cells to secrete chloride and/or other yet-to-be-discovered treatments. Sufficiency of treatment may be assessed, for example, by observing a decrease in the number of pathogenic organisms in sputum and/or lung lavage (such as Haemophilus influenzae, Stapholococcus aureus, and Pseudomonas aeruginosa), by monitoring the patient for weight gain, by detecting an increase in lung capacity or by any other convenient means.

The DAKAR polypeptides agonists and/or antagonists of the invention may be used for treating cystic fibrosis or fibrotic lung diseases, such as idiopathic pulmonary fibrosis, radiation-induced pulmonary fibrosis and bleomycin-induced pulmonary fibrosis. In addition, this combination is useful for treating other diseases characterized by organ fibrosis, including systemic sclerosis (also called “scleroderma”), which often involves fibrosis of the liver.

The DAKAR polypeptides and/or antagonists of the invention alone or in combination with other active compounds, such as IFNγ-1b, may be administered together with other treatments presently used for treating fibrotic lung disease. Such additional treatments include glucocorticoids, azathioprine, cyclophosphamide, penicillamine, colchisicine, supplemental oxygen and so forth. Patients with fibrotic lung disease, such as IPF, often present with nonproductive cough, progressive dyspnea, and show a restrictive ventilator pattern in pulmonary function tests. Chest radiographs reveal fibrotic accumulations in the patient's lungs. When treating fibrotic lung disease in accord with the disclosed methods, sufficiency of treatment can be detected by observing a decrease in the patient's coughing (when cough is present), or by using standard lung function tests to detect improvements in total lung capacity, vital capacity, residual lung volume or by administering a arterial blood gas determination measuring desaturation under exercising conditions, and showing that the patient's lung function has improved according to one or more of these measures. In addition, patient improvement can be determined through chest radiography results showing that the progression of fibrosis in the patient's lungs has become arrested or reduced.

In addition, DAKAR polypeptides and/or antagonists (including soluble DAKAR polypeptides and/or antibodies against DAKAR polypeptides, as well as small molecule mimetics) are useful for treating organ fibrosis when administered in combination with relaxin, a hormone that down-regulates collagen production thus inhibiting fibrosis, or when given in combination with agents that block the fibrogenic activity of TGF-β. Combination therapies using DAKAR polypeptides and/or antagonists and recombinant human relaxin are useful, for example, for treating systemic sclerosis and/or fibrotic lung diseases, including cystic fibrosis, idiopathic pulmonary fibrosis, radiation-induced pulmonary fibrosis and bleomycin-induced pulmonary fibrosis.

Other embodiments provide methods for using the disclosed DAKAR polypeptides and/or antagonists, compositions or combination therapies to treat a variety of rheumatic disorders. These include: adult and juvenile rheumatoid arthritis; systemic lupus erythematosus; gout; osteoarthritis; polymyalgia rheumatica; seronegative spondylarthropathies, including ankylosing spondylitis; and Reiter's disease. The subject DAKAR polypeptides and/or antagonists, compositions and combination therapies are used also to treat psoriatic arthritis and chronic Lyme arthritis. Also treatable with these compounds, compositions and combination therapies are Still's disease and uveitis associated with rheumatoid arthritis. In addition, the compounds, compositions and combination therapies of the invention are used in treating disorders resulting in inflammation of the voluntary muscle, including dermatomyositis and polymyositis. Moreover, the compounds, compositions and combinations disclosed herein are useful for treating sporadic inclusion body myositis, as DAKAR may play a significant role in the progression of this muscle disease. In addition, the compounds, compositions and combinations disclosed herein are used to treat multicentric reticulohistiocytosis, a disease in which joint destruction and papular nodules of the face and hands are associated with excess production of proinflammatory cytokines by multinucleated giant cells.

The DAKAR polypeptides and/or antagonists, compositions and combination therapies of the invention may be used to inhibit hypertrophic scarring, a phenomenon believed to result in part from excessive TNFα secretion. The DAKAR polypeptides and/or antagonists of the invention may be administered alone or concurrently with other agents that inhibit hypertrophic scarring.

Cervicogenic headache is a common form of headache arising from dysfunction in the neck area, and which is associated with elevated levels of TNFα, which are believed to mediate an inflammatory condition that contributes to the patient's discomfort (Martelletti, Clin Exp Rheumatol 18(2 Suppl 19):S33-8 (March-April, 2000)). Cervicogenic headache may be treated by administering DAKAR polypeptides and/or antagonists as disclosed herein, thereby reducing the inflammatory response and associated headache pain.

The DAKAR polypeptides and/or antagonists, compositions and combination therapies of the invention are useful for treating primary amyloidosis. In addition, the secondary amyloidosis that is characteristic of various conditions also are treatable with DAKAR polypeptides and/or antagonists such as DAKAR polypeptides and/or antagonists, and the compositions and combination therapies described herein. Such conditions include: Alzheimer's disease, secondary reactive amyloidosis; Down's syndrome; and dialysis-associated amyloidosis. Also treatable with the compounds, compositions and combination therapies of the invention are inherited periodic fever syndromes, including familial Mediterranean fever, hyperimmunoglobulin D and periodic fever syndrome and TNF-receptor associated periodic syndromes (TRAPS).

Disorders associated with transplantation also are treatable with the disclosed DAKAR polypeptides and/or antagonists, compositions or combination therapies, such as graft-versus-host disease, and complications resulting from solid organ transplantation, including transplantion of heart, liver, lung, skin, kidney and/or other organs. DAKAR polypeptides and/or antagonists may be administered, for example, to facilitate skin grafts and/or suppress differentiation of artificial skin grafts, as well as prevent or inhibit the development of bronchiolitis obliterans after lung transplantation.

Ocular disorders also are treatable with the disclosed DAKAR polypeptides agonists and/or antagonists, compositions or combination therapies, including rhegmatogenous retinal detachment, and inflammatory eye disease, and inflammatory eye disease associated with smoking and macular degeneration.

The DAKAR polypeptides and/or antagonists of the invention and the disclosed compositions and combination therapies also are useful for treating disorders that affect the female reproductive system. Examples include, but are not limited to, multiple implant failure/infertility; fetal loss syndrome and/or IV embryo loss (spontaneous abortion); preeclamptic pregnancies and/or eclampsia; and endometriosis.

In addition, the disclosed DAKAR polypeptides and/or antagonists, compositions and combination therapies are useful for treating obesity, including treatment to bring about a decrease in leptin formation. Also, the compounds, compositions and combination therapies of the invention are used to treat sciatica, symptoms of aging, severe drug reactions (for example, I1-2 toxicity and/or bleomycin-induced pneumopathy and fibrosis), and/or to suppress the inflammatory response prior, during and/or after the transfusion of allogeneic red blood cells in cardiac and/or other surgery, and/or in treating a traumatic injury to a limb and/or joint, such as traumatic knee injury. Various other medical disorders treatable with the disclosed DAKAR polypeptides and/or antagonists, compositions and combination therapies include: multiple sclerosis; Behcet's syndrome; Sjogren's syndrome; autoimmune hemolytic anemia; beta thalassemia; amyotrophic lateral sclerosis (Lou Gehrig's Disease); Parkinson's disease; and tenosynovitis of unknown cause, as well as various autoimmune disorders and/or diseases associated with hereditary deficiencies.

The disclosed DAKAR polypeptides and/or antagonists, compositions and combination therapies furthermore are useful for treating acute polyneuropathy; anorexia nervosa; Bell's palsy; chronic fatigue syndrome; transmissible dementia, including Creutzfeld-Jacob disease; demyelinating neuropathy; Guillain-Barre syndrome; vertebral disc disease; Gulf war syndrome; myasthenia gravis; silent cerebral ischemia; sleep disorders, including narcolepsy and sleep apnea; chronic neuronal degeneration; and stroke, including cerebral ischemic diseases.

The DAKAR polypeptides and/or antagonists of the invention may also exhibit one and/or more of the following additional activities and/or effects: inhibiting the growth, infection and/or function of, and/or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or circadian cycles or rhythms; effecting the fertility of male or female subjects; effecting the metabolism, catabolism, anabolism, processing, utilization, storage and/or elimination of dietary fat, lipid, polypeptide, carbohydrate, vitamins, minerals, cofactors and/or other nutritional factors and/or component(s); effecting behavioral characteristics, including, without limitation, appetite, libido, stress, cognition (including cognitive disorders), depression (including depressive disorders) and violent behaviors; providing analgesic effects and/or other pain reducing effects; promoting differentiation and growth of embryonic stem cells in lineages other than hematopoietic lineages; hormonal and/or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens and/or complement); and the ability to act as an antigen in a vaccine composition to raise an immune response against such polypeptide and/or another material and/or entity which is cross-reactive with such polypeptide.

Cardiovascular disorders are treatable with the disclosed DAKAR polypeptides agonists and/or antagonists, compositions or combination therapies, pharmaceutical compositions or combination therapies. Examples of cardiovascular disorders treatable with DAKAR, include: aortic aneurisms; arteritis; vascular occlusion, including cerebral artery occlusion; complications of coronary by-pass surgery; ischemia/reperfusion injury; heart disease, including atherosclerotic heart disease, myocarditis, including chronic autoimmune myocarditis and viral myocarditis; heart failure, including chronic heart failure (CHF), cachexia of heart failure; myocardial infarction; restenosis after heart surgery; silent myocardial ischemia; post-implantation complications of left ventricular assist devices; Raynaud's phenomena; thrombophlebitis; vasculitis, including Kawasaki's vasculitis; giant cell arteritis, Wegener's granulomatosis; and Schoenlein-Henoch purpura.

A combination of one or more DAKAR polypeptides agonists and/or antagonists, compositions or combination therapies and one or more other anti-angiogenesis factors may be used to treat solid tumors, thereby reducing the vascularization that nourishes the tumor tissue. Suitable anti-angiogenic factors for such combination therapies include IL-8 inhibitors, angiostatin, endostatin, kringle 5, inhibitors of vascular endothelial growth factor (VEGF), angiopoietin-2 or other antagonists of angiopoietin-1, antagonists of platelet-activating factor and antagonists of basic fibroblast growth factor. Antibodies against vascular endothelial growth factor, such as the recombinant humanized anti-VEGF produced by Genentech, Inc., are useful for combination treatments with TNFα inhibitors such as TNFR:Fc.

Administration of DAKAR Polypeptides, Agonists and Antagonists Thereof

This invention provides compounds, compositions, and methods for treating a patient, preferably a mammalian patient, and most preferably a human patient, who is suffering from a medical disorder, and in particular a DAKAR-mediated disorder. Such DAKAR-mediated disorders include conditions caused (directly or indirectly) or exacerbated by binding between DAKAR and a binding partner. For purposes of this disclosure, the terms “illness,” “disease,” “medical condition,” “abnormal condition” and the like are used interchangeably with the term “medical disorder.” The terms “treat”, “treating”, and “treatment” used herein includes curative, preventative (e.g., prophylactic) and palliative or ameliorative treatment. For such therapeutic uses, DAKAR polypeptides and fragments, DAKAR nucleic acids encoding the DAKAR family polypeptides, and/or agonists or antagonists of the DAKAR polypeptide such as antibodies can be administered to the patient in need through well-known means. Compositions of the present invention can contain a polypeptide in any form described herein, such as native polypeptides, variants, derivatives, oligomers, and biologically active fragments. In particular embodiments, the composition comprises a soluble polypeptide or an oligomer comprising soluble DAKAR polypeptides.

Therapeutically Effective Amount. In practicing the method of treatment or use of the present invention, a therapeutically effective amount of a therapeutic agent of the present invention is administered to a patient having a condition to be treated, preferably to treat or ameliorate diseases associated with the activity of a DAKAR family polypeptide. “Therapeutic agent” includes without limitation any of the DAKAR polypeptides, fragments, and variants; nucleic acids encoding the DAKAR family polypeptides, fragments, and variants; agonists or antagonists of the DAKAR polypeptides such as antibodies; DAKAR polypeptide binding partners; complexes formed from the DAKAR family polypeptides, fragments, variants, and binding partners, etc. As used herein, the term “therapeutically effective amount” means the total amount of each therapeutic agent or other active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual therapeutic agent or active ingredient, administered alone, the term refers to that ingredient alone. When applied to a combination, the term refers to combined amounts of the ingredients that result in the therapeutic effect, whether administered in combination, serially or simultaneously. As used herein, the phrase “administering a therapeutically effective amount” of a therapeutic agent means that the patient is treated with said therapeutic agent in an amount and for a time sufficient to induce an improvement, and preferably a sustained improvement, in at least one indicator that reflects the severity of the disorder. An improvement is considered “sustained” if the patient exhibits the improvement on at least two occasions separated by one or more days, or more preferably, by one or more weeks. The degree of improvement is determined based on signs or symptoms, and determinations can also employ questionnaires that are administered to the patient, such as quality-of-life questionnaires. Various indicators that reflect the extent of the patient's illness can be assessed for determining whether the amount and time of the treatment is sufficient. The baseline value for the chosen indicator or indicators is established by examination of the patient prior to administration of the first dose of the therapeutic agent. Preferably, the baseline examination is done within about 60 days of administering the first dose. If the therapeutic agent is being administered to treat acute symptoms, the first dose is administered as soon as practically possible after the injury has occurred. Improvement is induced by administering therapeutic agents such as DAKAR polypeptides and/or antagonists until the patient manifests an improvement over baseline for the chosen indicator or indicators. In treating chronic conditions, this degree of improvement is obtained by repeatedly administering this medicament over a period of at least a month or more, e.g., for one, two, or three months or longer, or indefinitely. A period of one to six weeks, or even a single dose, often is sufficient for treating injuries or other acute conditions. Although the extent of the patient's illness after treatment may appear improved according to one or more indicators, treatment may be continued indefinitely at the same level or at a reduced dose or frequency. Once treatment has been reduced or discontinued, it later may be resumed at the original level if symptoms should reappear.

Dosing. One skilled in the pertinent art will recognize that suitable dosages will vary, depending upon such factors as the nature and severity of the disorder to be treated, the patient's body weight, age, general condition, and prior illnesses and/or treatments, and the route of administration. Preliminary doses can be determined according to animal tests, and the scaling of dosages for human administration is performed according to art-accepted practices such as standard dosing trials. For example, the therapeutically effective dose can be estimated initially from cell culture assays. The dosage will depend on the specific activity of the compound and can be readily determined by routine experimentation. A dose can be formulated in animal models to achieve a circulating plasma concentration range that includes the IC50 (i.e., the concentration of the test compound which achieves a half-maximal inhibition of symptoms) as determined in cell culture, while minimizing toxicities. Such information can be used to more accurately determine useful doses in humans. Ultimately, the attending physician will decide the amount of polypeptide of the present invention with which to treat each individual patient. Initially, the attending physician will administer low doses of polypeptide of the present invention and observe the patient's response. Larger doses of polypeptide of the present invention can be administered until the optimal therapeutic effect is obtained for the patient, and at that point the dosage is not increased further. It is contemplated that the various pharmaceutical compositions used to practice the method of the present invention should contain about 0.01 ng to about 100 mg (preferably about 0.1 ng to about 10 mg, more preferably about 0.1 microgram to about 1 mg) of polypeptide of the present invention per kg body weight. In one embodiment of the invention, DAKAR polypeptides and/or antagonists are administered one time per week to treat the various medical disorders disclosed herein, in another embodiment is administered at least two times per week, and in another embodiment is administered at least three times per week. If injected, the effective amount of DAKAR polypeptides and/or antagonists per adult dose ranges from 1-20 mg/m², and preferably is about 5-12 mg/m². Alternatively, a flat dose can be administered, whose amount may range from 5-100 mg/dose. Exemplary dose ranges for a flat dose to be administered by subcutaneous injection are 5-25 mg/dose, 25-50 mg/dose and 50-100 mg/dose. In one embodiment of the invention, the various indications described below are treated by administering a preparation acceptable for injection containing DAKAR polypeptides and/or antagonists at 25 mg/dose, or alternatively, containing 50 mg per dose. The 25 mg or 50 mg dose can be administered repeatedly, particularly for chronic conditions. If a route of administration other than injection is used, the dose is appropriately adjusted in accord with standard medical practices. In many instances, an improvement in a patient's condition will be obtained by injecting a dose of about 25 mg of DAKAR polypeptides and/or antagonists one to three times per week over a period of at least three weeks, or a dose of 50 mg of DAKAR polypeptides and/or antagonists one or two times per week for at least three weeks, though treatment for longer periods may be necessary to induce the desired degree of improvement. For incurable chronic conditions, the regimen can be continued indefinitely, with adjustments being made to dose and frequency if such are deemed necessary by the patient's physician. The foregoing doses are examples for an adult patient who is a person who is 18 years of age or older. For pediatric patients (age 4-17), a suitable regimen involves the subcutaneous injection of 0.4 mg/kg, up to a maximum dose of 25 mg of DAKAR polypeptides and/or antagonists, administered by subcutaneous injection one or more times per week. If an antibody against a DAKAR polypeptide is used as the DAKAR polypeptide antagonist, a preferred dose range is 0.1 to 20 mg/kg, and more preferably is 1-10 mg/kg. Another preferred dose range for an anti-DAKAR polypeptide antibody is 0.75 to 7.5 mg/kg of body weight. Humanized antibodies are preferred, that is, antibodies in which only the antigen-binding portion of the antibody molecule is derived from a non-human source. Such antibodies can be injected or administered intravenously.

Formulations. Compositions comprising an effective amount of a DAKAR polypeptide of the present invention (from whatever source derived, including without limitation from recombinant and non-recombinant sources), in combination with other components such as a physiologically acceptable diluent, carrier, or excipient, are provided herein. The term “pharmaceutically acceptable” means a non-toxic material that does not interfere with the effectiveness of the biological activity of the active ingredient(s). Formulations suitable for administration include aqueous and non-aqueous sterile injection solutions which can contain anti-oxidants, buffers, bacteriostats and solutes which render the formulation isotonic with the blood of the recipient; and aqueous and non-aqueous sterile suspensions which can include suspending agents or thickening agents. The polypeptides can be formulated according to known methods used to prepare pharmaceutically useful compositions. They can be combined in admixture, either as the sole active material or with other known active materials suitable for a given indication, with pharmaceutically acceptable diluents (e.g., saline, Tris-HCl, acetate, and phosphate buffered solutions), preservatives (e.g., thimerosal, benzyl alcohol, parabens), emulsifiers, solubilizers, adjuvants and/or carriers. Suitable formulations for pharmaceutical compositions include those described in Remington's Pharmaceutical Sciences, 16th ed. 1980, Mack Publishing Company, Easton, Pa. In addition, such compositions can be complexed with polyethylene glycol (PEG), metal ions, or incorporated into polymeric compounds such as polyacetic acid, polyglycolic acid, hydrogels, dextran, etc., or incorporated into liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323. Such compositions will influence the physical state, solubility, stability, rate of in vivo release, and rate of in vivo clearance, and are thus chosen according to the intended application, so that the characteristics of the carrier will depend on the selected route of administration. In one preferred embodiment of the invention, sustained-release forms of DAKAR polypeptides are used. Sustained-release forms suitable for use in the disclosed methods include, but are not limited to, DAKAR polypeptides that are encapsulated in a slowly-dissolving biocompatible polymer (such as the alginate microparticles described in U.S. No. 6,036,978), admixed with such a polymer (including topically applied hydrogels), and or encased in a biocompatible semi-permeable implant.

Combinations of Therapeutic Compounds. A DAKAR polypeptide of the present invention may be active in multimers (e.g., heterodimers or homodimers) or complexes with itself or other polypeptides. As a result, pharmaceutical compositions of the invention may comprise a polypeptide of the invention in such multimeric or complexed form. The pharmaceutical composition of the invention may be in the form of a complex of the polypeptide(s) of present invention along with polypeptide or peptide antigens. The invention further includes the administration of DAKAR polypeptides and/or antagonists concurrently with one or more other drugs that are administered to the same patient in combination with the DAKAR polypeptides and/or antagonists, each drug being administered according to a regimen suitable for that medicament. “Concurrent administration” encompasses simultaneous and/or sequential treatment with the components of the combination, as well as regimens in which the drugs are alternated, or wherein one component is administered long-term and the other(s) are administered intermittently. Components can be administered in the same or in separate compositions, and by the same or different routes of administration. Examples of components that can be administered concurrently with the pharmaceutical compositions of the invention are: cytokines, lymphokines, or other hematopoietic factors such as M-CSF, GM-CSF, TNF, IL-1, IL-2, IL-3, IL4, IL-5, IL-6, IL-7, IL-8, IL-9, IL-10, IL-1, IL-12, IL-13, IL-14, IL-15, IL-17, IL-18, IFN, TNF0, TNF1, TNF2, G-CSF, Meg-CSF, thrombopoietin, stem cell factor, and erythropoietin, or inhibitors or antagonists of any of these factors. The pharmaceutical composition can further contain other agents which either enhance the activity of the polypeptide or compliment its activity or use in treatment. Such additional factors and/or agents may be included in the pharmaceutical composition to produce a synergistic effect with polypeptide of the invention, or to minimize side effects. Conversely, a DAKAR polypeptide or antagonist of the present invention may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent. Additional examples of drugs to be administered concurrently include but are not limited to antivirals, antibiotics, analgesics, corticosteroids, antagonists of inflammatory cytokines, non-steroidal anti-inflammatories, pentoxifylline, thalidomide, and disease-modifying antirheumatic drugs (DMARDs) such as azathioprine, cyclophosphamide, cyclosporine, hydroxychloroquine sulfate, methotrexate, leflunomide, minocycline, penicillamine, sulfasalazine and gold compounds such as oral gold, gold sodium thiomalate, and aurothioglucose. Additionally, DAKAR polypeptides and/or antagonists can be combined with a second DAKAR polypeptide/antagonist, including an antibody against a DAKAR polypeptide, or a DAKAR polypeptide-derived peptide that acts as a competitive inhibitor of a native DAKAR polypeptide.

Routes of Administration. Any efficacious route of administration can be used to therapeutically administer DAKAR polypeptides and/or antagonists thereof, including those compositions comprising nucleic acids. Parenteral administration includes injection, for example, via intra-articular, intravenous, intramuscular, intralesional, intraperitoneal or subcutaneous routes by bolus injection or by continuous infusion., and also includes localized administration, e.g., at a site of disease or injury. Other suitable means of administration include sustained release from implants; aerosol inhalation and/or insufflation.; eyedrops; vaginal or rectal suppositories; buccal preparations; oral preparations, including pills, syrups, lozenges, ice creams, or chewing gum; and topical preparations such as lotions, gels, sprays, ointments or other suitable techniques. Alternatively, polypeptideaceous DAKAR polypeptides and/or antagonists may be administered by implanting cultured cells that express the polypeptide, for example, by implanting cells that express DAKAR polypeptides and/or antagonists. Cells may also be cultured ex vivo in the presence of polypeptides of the present invention in order to modulate cell proliferation or to produce a desired effect on or activity in such cells. Treated cells can then be introduced in vivo for therapeutic purposes. The polypeptide of the instant invention may also be administered by the method of protein transduction. In this method, the DAKAR polypeptide is covalently linked to a protein-transduction domain (PTD) such as, but not limited to, TAT, Antp, or VP22 (Schwarze et al., 2000, Cell Biology 10:290-295). The PTD-linked peptides can then be transduced into cells by adding the peptides to tissue-culture media containing the cells (Schwarze et al., 1999, Science 285:1569; Lindgren et al., 2000, TiPS 21:99; Derossi et al., 1998, Cell Biology 8:84; WO 00/34308; WO 99/29721; and WO 99/10376). In another embodiment, the patient's own cells are induced to produce DAKAR polypeptides and/or antagonists by transfection in vivo or ex vivo with a DNA that encodes DAKAR polypeptides and/or antagonists. This DNA can be introduced into the patient's cells, for example, by injecting naked DNA or liposome-encapsulated DNA that encodes DAKAR polypeptides, and/or antagonists, or by other means of transfection. Nucleic acids of the invention can also be administered to patients by other known methods for introduction of nucleic acid into a cell or organism (including, without limitation, in the form of viral vectors or naked DNA). When DAKAR polypeptides and/or antagonists are administered in combination with one or more other biologically active compounds, these can be administered by the same or by different routes, and can be administered simultaneously, separately or sequentially.

Oral Administration. When a therapeutically effective amount of polypeptide of the present invention is administered orally, polypeptide of the present invention will be in the form of a tablet, capsule, powder, solution or elixir. When administered in tablet form, the pharmaceutical composition of the invention can additionally contain a solid carrier such as a gelatin or an adjuvant. The tablet, capsule, and powder contain from about 5 to 95% polypeptide of the present invention, and preferably from about 25 to 90% polypeptide of the present invention. When administered in liquid form, a liquid carrier such as water, petroleum, oils of animal or plant origin such as peanut oil, mineral oil, soybean oil, or sesame oil, or synthetic oils can be added. The liquid form of the pharmaceutical composition can further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of polypeptide of the present invention, and preferably from about 1 to 50% polypeptide of the present invention.

Intravenous Administration. When a therapeutically effective amount of polypeptide of the present invention is administered by intravenous, cutaneous or subcutaneous injection, polypeptide of the present invention will be in the form of a pyrogen-free, parenterally acceptable aqueous solution. The preparation of such parenterally acceptable polypeptide solutions, having due regard to pH, isotonicity, stability, and the like, is within the skill in the art. A preferred pharmaceutical composition for intravenous, cutaneous, or subcutaneous injection should contain, in addition to polypeptide of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention can also contain stabilizers, preservatives, buffers, antioxidants, or other additives known to those of skill in the art. The duration of intravenous therapy using the pharmaceutical composition of the present invention will vary, depending on the severity of the disease being treated and the condition and potential idiosyncratic response of each individual patient. It is contemplated that the duration of each application of the polypeptide of the present invention will be in the range of 12 to 24 hours of continuous intravenous administration. Ultimately the attending physician will decide on the appropriate duration of intravenous therapy using the pharmaceutical composition of the present invention.

Bone and Tissue Administration. For compositions of the present invention which are useful for bone, cartilage, tendon or ligament disorders, the therapeutic method includes administering the composition topically, systematically, or locally as an implant or device. When administered, the therapeutic composition for use in this invention is, of course, in a pyrogen-free, physiologically acceptable form. Further, the composition can desirably be encapsulated or injected in a viscous form for delivery to the site of bone, cartilage or tissue damage. Topical administration may be suitable for wound healing and tissue repair. Therapeutically useful agents other than a polypeptide of the invention which may also optionally be included in the composition as described above, can alternatively or additionally, be administered simultaneously or sequentially with the composition in the methods of the invention. Preferably for bone and/or cartilage formation, the composition would include a matrix capable of delivering the polypeptide-containing composition to the site of bone and/or cartilage damage, providing a structure for the developing bone and cartilage and optimally capable of being resorbed into the body. Such matrices can be formed of materials presently in use for other implanted medical applications. The choice of matrix material is based on biocompatibility, biodegradability, mechanical properties, cosmetic appearance and interface properties. The particular application of the compositions will define the appropriate formulation. Potential matrices for the compositions can be biodegradable and chemically defined calcium sulfate, tricalciumphosphate, hydroxyapatite, polylactic acid, polyglycolic acid and polyanhydrides. Other potential materials are biodegradable and biologically well-defined, such as bone or dermal collagen. Further matrices are comprised of pure polypeptides and/or extracellular matrix components. Other potential matrices are nonbiodegradable and chemically defined, such as sintered hydroxapatite, bioglass, aluminates, or other ceramics. Matrices can be comprised of combinations of any of the above mentioned types of material, such as polylactic acid and hydroxyapatite or collagen and tricalciumphosphate. The bioceramics can be altered in composition, such as in calcium-aluminate-phosphate and processing to alter pore size, particle size, particle shape, and biodegradability. Presently preferred is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the polypeptide compositions from disassociating from the matrix. A preferred family of sequestering agents is cellulosic materials such as alkylcelluloses (including hydroxyalkylcelluloses), including methylcellulose, ethylcellulose, hydroxyethylcellulose, hydroxypropylcellulose, hydroxypropyl-methylcellulose, and carboxymethyl-cellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 0.5-20 wt %, preferably 1-10 wt % based on total formulation weight, which represents the amount necessary to prevent desorbtion of the polypeptide from the polymer matrix and to provide appropriate handling of the composition, yet not so much that the progenitor cells are prevented from infiltrating the matrix, thereby providing the polypeptide the opportunity to assist the osteogenic activity of the progenitor cells. In further compositions, polypeptides of the invention may be combined with other agents beneficial to the treatment of the bone and/or cartilage defect, wound, or tissue in question. These agents include various growth factors such as epidermal growth factor (EGF), platelet derived growth factor (PDGF), transforming growth factors (TGF-alpha and TGF-beta), and insulin-like growth factor (IGF). The therapeutic compositions are also presently valuable for veterinary applications. Particularly domestic animals and thoroughbred horses, in addition to humans, are desired patients for such treatment with polypeptides of the present invention. The dosage regimen of a polypeptide-containing pharmaceutical composition to be used in tissue regeneration will be determined by the attending physician considering various factors which modify the action of the polypeptides, e.g., amount of tissue weight desired to be formed, the site of damage, the condition of the damaged tissue, the size of a wound, type of damaged tissue (e.g., bone), the patient's age, sex, and diet, the severity of any infection, time of administration and other clinical factors. The dosage can vary with the type of matrix used in the reconstitution and with inclusion of other polypeptides in the pharmaceutical composition. For example, the addition of other known growth factors, such as IGF I (insulin like growth factor I), to the final composition, may also effect the dosage. Progress can be monitored by periodic assessment of tissue/bone growth and/or repair, for example, X-rays, histomorphometric determinations and tetracycline labeling.

Veterinary Uses. In addition to human patients, DAKAR polypeptides and antagonists are useful in the treatment of disease conditions in non-human animals, such as pets (dogs, cats, birds, primates, etc.), domestic farm animals (horses cattle, sheep, pigs, birds, etc.), or any animal that suffers from a DAKAR-mediated condition. In such instances, an appropriate dose can be determined according to the animal's body weight. For example, a dose of 0.2-1 mg/kg may be used. Alternatively, the dose is determined according to the animal's surface area, an exemplary dose ranging from 0. 1-20 mg/m², or more preferably, from 5-12 mg/m². For small animals, such as dogs or cats, a suitable dose is 0.4 mg/kg. In a preferred embodiment, DAKAR polypeptides and/or antagonists (preferably constructed from genes derived from the same species as the patient), is administered by injection or other suitable route one or more times per week until the animal's condition is improved, or it can be administered indefinitely.

Manufacture of Medicaments. The present invention also relates to the use of DAKAR polypeptides, fragments, and variants; nucleic acids encoding the DAKAR family polypeptides, fragments, and variants; agonists or antagonists of the DAKAR polypeptides such as antibodies; DAKAR polypeptide binding partners; complexes formed from the DAKAR family polypeptides, fragments, variants, and binding partners, etc, in the manufacture of a medicament for the prevention or therapeutic treatment of each medical disorder disclosed herein.

Use of DAKAR Polypeptides and Antagonists Thereof as Adjuvants

An effective vaccine must induce an appropriate immune response to the correct antigen or antigens. The immune system uses many mechanisms for attacking pathogens, but not all of these are activated after immunization. Protective immunity induced by vaccination is dependent on the capacity of the vaccine to elicit the appropriate immune response to resist, control, or eliminate the pathogen. Depending on the pathogen, this may require a humoral immune response, which involves antibodies and other factors such as complement, and/or a cell-mediated immune response, which is mediated by cells such as cytotoxic T cells. The type of immune response that is produced is determined by the nature of the T cells that develop after immunization. For example, many bacterial, protozoal, and intracellular parasitic and viral infections appear to require a strong cell-mediated immune response for protection, while other pathogens such as helminths primarily respond to a humoral response. The current paradigm of the role of T cells in the particular immune response is that CD4⁺T cells can be separated into subsets on the basis of the repertoire of cytokines produced and that the distinct cytokine profile observed in these cells determines their function. This T cell model includes two major subsets: Th1 cells that produce IL-2 and interferon gamma (IFN-gamma) and mediate cellular immune responses, and Th2 cells that produce IL-4, IL-5, and IL-10 and augment humoral immune responses (Mosmann et al., 1986, J Immunol 126:2348).

Many vaccine compositions employ adjuvants, that is, substances which enhance the immune response when administered together with an immunogen or antigen. Adjuvants are thought to function in one or more of several possible ways, including increasing the surface area of antigen; prolonging the retention of the antigen in the body thus allowing time for the lymphoid system to have access to the antigen; slowing the release of antigen; targeting antigen to macrophages; increasing antigen uptake; up-regulating antigen processing; stimulating cytokine release; stimulating B cell switching and maturation and/or eliminating immuno-suppressor cells; activating macrophages, dendritic cells, B cells and T cells; or otherwise eliciting non-specific activation of the cells of the immune system (see, for example, Warren et al., 1986, Annu Rev Immunol 4:369). Many of the most effective adjuvants include bacteria or their products, e.g., microorganisms such as the attenuated strain of Mycobacterium bovis, bacillus Calmette-Guerin (BCG); microorganism components, e.g., alum-precipitated diphtheria toxoid, bacterial lipopolysaccharide and endotoxins. Despite their immunostimulating properties, many bacterial adjuvants have toxic or other negative effects, particularly in humans. For example, such a large population has been exposed to some of the bacterial adjuvants, like BCG, that there is a danger of eliciting a secondary response with future use as a vaccine adjuvant. Heat-killed bacteria, being non-native to mammalian hosts, also risk causing toxic effects in the host. Alternative adjuvants that stimulate or enhance the host's immune responses without inducing a toxic effect, and which are suitable for use in pharmaceutical compositions, such as vaccines, are particularly useful. Also, an essential role of adjuvants in vaccines is to modulate CD4⁺ T cell subset differentiation. The ability of an adjuvant to induce and increase a specific type of effector T cell (Th1 or Th2) and thus a specific type of immune response (cell-mediated or humoral) is a key factor in the selection of particular adjuvants for vaccine use against a particular pathogen. The present invention provides the use of DAKAR polypeptides and agonists thereof as adjuvants in vaccines, in order to promote the production of Th1 and/or Th2 cells by the vaccine, and/or to increase or modify the immunogenicity or tolerance-inducing activity of the vaccine, which is useful for example when the vaccine is meant to increase tolerance toward an allergenic antigen. Also provided by the present invention is the use of antagonists of DAKAR polypeptide activity as adjuvants in vaccines, in order to promote the production of Th1 and/or Th2 cells by the vaccine, and/or to increase or modify the immunogenicity or tolerance-inducing activity of the vaccine, which is useful for example when the vaccine is meant to increase tolerance toward an allergenic antigen.

Antigens are substances which are capable, under appropriate conditions, of inducing a specific immune response and of reacting with the products of that response, such as specific antibodies or T cells, or both. A vaccine is a composition comprising antigenic moieties, usually consisting of inactivated infectious agents or of allergens, or some part of an infectious agent or allergen, that is injected into the body to produce active immunity, or in the case of allergens, to induce tolerance. Antigens that can be used in the present invention are compounds which, when introduced into a mammal, preferably a human, will result in the formation of antibodies and/or cell-mediated immunity. Representative of the antigens that can be used according to the present invention include, but are not limited to live or killed viruses and other microorganisms; natural, recombinant or synthetic products derived from viruses, bacteria, fungi, parasites and other infectious agents; antigens promoting autoimmune diseases, hormones, or tumor antigens which might be used in prophylactic or therapeutic vaccines; and allergens (see Table 1 below). The viral or microorganism products can be components which are produced by enzymatic cleavage or can be components of the organism (proteins, polypeptides, polysaccharides, nucleic acids, lipids, etc.) that were produced by recombinant DNA techniques that are well-known to those of ordinary skill in the art. The antigen component of the vaccine may also comprise one or several antigenic molecules such as haptens, which are small antigenic determinants capable of eliciting an immune response only when coupled to a carrier.

TABLE 1

| Antigen | |
|---|---|
| Category | Some Specific Examples of Representative Antigens |
| Viruses | Rotavirus; foot and mouth disease; influenza, including influenza |
| | A and B; parainfluenza; Herpes species (Herpes simplex, |
| | Epstein-Barr virus, chicken pox, pseudorabies, cytomegalovirus); |
| | rabies; polio; hepatitis A; hepatitis B; hepatitis C; hepatitis E; |
| | measles; distemper; Venezuelan equine encephalomyelitis; feline |
| | leukemia virus; reovirus; respiratory syncytial virus; bovine |
| | respiratory syncytial virus; Lassa fever virus; polyoma tumor |
| | virus; parvovirus; canine parvovirus; papilloma virus; tick-borne |
| | encephalitis; rinderpest; human rhinovirus species; enterovirus |
| | species; Mengo virus; paramyxovirus; avian infectious bronchitis |
| | virus; HTLV 1; HIV-1; HIV-2; LCMV (lymphocytic |
| | choriomeningitis virus); adenovirus; togavirus (rubella, yellow |
| | fever, dengue fever); corona virus |
| Bacteria | Bordetella pertussis; Brucella abortis; Escherichia coli; |
| | Salmonella species including Salmonella typhi; streptococci; |
| | Vibrio species (V. cholera, V. parahaemolyticus); Shigella |
| | species; Pseudomonas species; Brucella species; Mycobacteria |
| | species (tuberculosis, avium, BCG, leprosy); pneumococci; |
| | staphlylococci; Enterobacter species; Rochalimaia henselae; |
| | Pasterurella species (P. haemolytica, P. multocida); Chlamydia |
| | species (C. trachomatis, C. psittac; Lymphogranuloma |
| | venereum); Syphilis (Treponema pallidum); Haemophilus |
| | species; Mycoplasma species; Lyme disease (Borrelia |
| | burgdorferi); Legionnaires' disease; Botulism (Colstridium |
| | botulinum); Corynebacterium diphtheriae; Yersinia entercolitica |
| Ricketsial | Rocky mountain spotted fever; thyphus; Ehrlichia species |
| Infections | |
| Parasites and | Malaria (Plasmodium falciparum, P. vivax, P. malariae); |
| Protozoa | schistosomes; trypanosomes; Leishmania species; filarial |
| | nematodes; trichomoniasis; sarcosporidiasis; Taenia species (T. |
| | saginata, T. solium); Toxoplasma gondii; trichinelosis |
| | (Trichinella spiralis); coccidiosis (Eimeria species) |
| Fungi | Cryptococcus neoformans; Candida albicans; Apergillus |
| | fumigatus; coccidioidomycosis |
| Recombinant | Herpes simplex; Epstein-Barr virus; hepatitis B; pseudorabies; |
| Proteins | flavivirus (dengue, yellow fever); Neisseria gonorrhoeae; |
| | malaria: circumsporozoite protein, merozoite protein; |
| | trypanosome surface antigen protein; pertussis; alphaviruses; |
| | adenovirus |
| Proteins | Diphtheria toxoid; tetanus toxoid; meningococcal outer |
| | membrane protein (OMP); streptococcal M protein; hepatitis B; |
| | influenza hemagglutinin; cancer antigen; tumor antigens; toxins; |
| | exotoxins; neurotoxins; cytokines and cytokine receptors; |
| | monokines and monokine receptors |
| Synthetic | Malaria; influenza; foot and mouth disease virus; hepatitis B; |
| Peptides | hepatitis C |
| Poly-saccharides | Pneumococcal polysaccharide; Haemophilis influenza |
| | polyribosyl-ribitolphosphate (PRP); Neisseria meningitides; |
| | Pseudomonas aeruginosa; Klebsiella pneumoniae |
| Oligo-saccharide | Pneumococcal |
| Allergens | Plant pollens; animal dander; dust mites, Blatella species |
| | antigens (Bla g 1, 2, or 5), Periplaneta species antigens (Per a 1) |

Adjuvants are compounds that, when used in combination with specific vaccine antigens, augment or otherwise alter or modify the resultant immune responses. Modification of the immune response means augmenting, intensifying, or broadening the specificity of either or both antibody and cellular immune responses. Modification of the immune response can also mean decreasing or suppressing certain antigen-specific immune responses, for example, in the induction of tolerance toward an allergen. Modification of the immune response by the adjuvant may increase the overall titer of antibodies specific for the vaccine antigen and/or induce cellular immune responses specific for the vaccine antigen, so that effective vaccination can be made using lower amounts of antigen. Methods for detecting modification of the immune response by the adjuvant include several well-known assays such as ELISA (enzyme-linked immunosorbent assay), which measures the titer of antigen-specific antibodies, and the ELISPOT (enzyme-linked immunospot) assay, which allows ex vivo quantification of antigen-reactive T cells and of cells producing antigen-specific antibodies (see, for example, Zigterman et al., 1988, J Immunol Methods 106:101-107; U.S. Pat. No. 6,149,922; and U.S. Pat. No. 6,153,182). Variations of ELISA in which biotin/avidin interactions are used to create antibody-antigen-antibody ‘bridges’ or ‘sandwiches’ are also well known in the art (see, for example, U.S. Pat. No. 6,149,922). In order to measure the effect of an adjuvant preparation on the production of functional, neutralizing antibodies, influenza virus hemagglutinin (HA) can be used as an antigen, animals are immunized with HA with differing amounts of adjuvant, and the ability of the resulting serum antibodies to inhibit the hemagglutinin-dependent agglutination of red blood cells can be determined using a hemagglutination inhibition (HAI) assay, essentially as described by the CDC Manual (U.S. Department of Health and Human Services/Public Health Service/Centers for Disease Control, 1982, Concepts and Procedures for Laboratory Based Influenza Surveillance) and U.S. Pat. No. 6,149,922. These assays allow the effects of supplementing a vaccine with DAKAR polypeptides and/or antagonists to be investigated by determining antibody titers and the kinetics of antibody responses. For example, dose-titration studies of a vaccine can be done to identify doses that induce measurable antibody responses after a single immunization. Antibody responses are followed for 30, 60, or 90 or more days and dose levels that are optimally and suboptimally immunogenic can be identified. Also, vaccine formulations containing these dose levels and supplemented with increasing amounts of adjuvant (DAKAR polypeptide or antagonist) can be evaluated and active doses of adjuvant identified. The kinetics and duration of antibody responses can evaluated by extension of the observation and antibody testing period to 6 months or more (see, for example, U.S. Pat. No. 6,149,922). Modulation of the immune response by adjuvant can also be assessed by measuring the antigen-dependent proliferation of T cells from immunized mice in a ³H-thymidine uptake assay (see, for example, U.S. Pat. No. 6,051,227 and U.S. Pat. No. 6,153,182). Other T cell responses to immunization with varying amounts of adjuvant can be measured by determining the profile of cytokines secreted by T cells isolated from immunized animals, which may indicate whether Th1 or Th2 effector T cells are preferentially produced, or by assaying for functional cytotoxic T cells (see, for example, U.S. Pat. No. 6,149,922).

When used as an adjuvant in a vaccine composition, DAKAR polypeptides and/or antagonists are desirably admixed as part of the vaccine composition itself. One of skill in the art of vaccine composition can readily determine suitable amounts of DAKAR polypeptides and/or antagonists to adjuvant particular vaccines. Such amounts will depend upon the purpose for which the vaccine is designed, the nature of the antigen, and the dosage amounts of the antigen, as well as the species and physical and medical conditions of the vaccinate. As one example, an effective adjuvant amount of a DAKAR polypeptide or antagonist is desirably between about 0.01 micrograms to about 10 mg (preferably about 0.1 microgram to about 1 mg, and more preferably about 1 microgram to about 0.1 mg) of DAKAR polypeptide or antagonist per about 25 micrograms of antigen. When administered as part of a vaccine composition, DAKAR polypeptides and/or antagonists are administered by the same route as the vaccine antigen. Any route of administration can be employed for the administration of this vaccine, e.g., subcutaneous, intraperitoneal, oral, intramuscular, intranasal and the like. The adjuvants may be given orally in alkaline solutions in vaccines appropriate for raising mucosal antibodies against antigens which give rise to intestinal diseases, as alkaline solutions such as those containing bicarbonates protect antigens and adjuvants from destruction in the upper GI tract. Alternatively, the adjuvant effect of DAKAR polypeptides and/or antagonists can be employed by administering DAKAR polypeptides and/or antagonists separately from the vaccine composition, and preferably in the presence of a suitable carrier, such as saline and optionally conventional pharmaceutical agents enabling gradual release of the DAKAR polypeptide or antagonist. The amount of the DAKAR polypeptides and/or antagonists used in this mode of vaccination is similar to the ranges identified above when DAKAR polypeptides and/or antagonists are part of the vaccine composition. The DAKAR polypeptides and/or antagonists can be administered contemporaneously with the vaccine composition, either simultaneously therewith, or before the vaccine antigen administration. If the DAKAR polypeptide or antagonist is administered before the vaccine composition, it is desirable to administer it about one or more days before the vaccine. When DAKAR polypeptides and/or antagonists are administered as a separate component from the vaccine, they are desirably administered by the same route as the vaccine antigen, e.g., subcutaneous route, or any other route as selected by a physician.

In addition to the administration of DAKAR polypeptides and/or antagonists as an adjuvant, nucleic acid sequences encoding DAKAR polypeptides and/or antagonists or a fragment thereof can also be used as an adjuvant. The nucleic acid sequences, preferably in the form of DNA, can be delivered to a vaccinate for in vivo expression of the DAKAR polypeptide or antagonist. Naked DNA can also be used to express the DAKAR polypeptides and/or antagonists in a patient (see, for example, Cohen, 1993, Science 259:1691-1692; Fynan et al., 1993, Proc Natl Acad Sci 90:11478-11482; and Wolff et al., 1991, Biotechniques 11:474-485). For example, DAKAR DNA can be incorporated into a microorganism itself, if it as a whole pathogen is to be employed as the vaccine antigen. Alternatively, DAKAR DNA can be administered as part of the vaccine composition or separately, but contemporaneously with the vaccine antigen, e.g., by injection. Still other modes of delivering DAKAR polypeptide or antagonist to the vaccinate in the form of DNA are known to those of skill in the art and can be employed rather than administration of the DAKAR polypeptide or antagonist, as desired. For example, DAKAR DNA can be administered as part of a vector or as a cassette containing the DAKAR DNA sequences operatively linked to a promoter sequence. When DAKAR nucleic acid sequences are used as an adjuvant, these sequences can be operably linked to DNA sequences which encode the antigen. Hence, the vector or cassette, as described above, encoding the DAKAR DNA sequences can additionally include sequences encoding the antigen. Each of these sequences can be operatively linked to the promoter sequence of the vector or cassette. Alternatively, naked DNA encoding the antigen can be in a separate plasmid. Where present in one or two plasmids, the naked DNA encoding the antigen and/or DAKAR polypeptide or antagonist, upon introduction into the host cells, permits the infection of the vaccinate's cells and expression of both antigen and DAKAR polypeptide or antagonist in vivo. When DAKAR nucleic acid sequences are employed as the adjuvant, the amounts of DNA to be delivered and the routes of delivery may parallel the DAKAR polypeptide or antagonist amounts and delivery described above, and can also be determined readily by one of skill in the art. Similarly the amounts of the antigen-encoding DNA can be selected by one of skill in the art.

### EXAMPLES

The following examples are intended to illustrate particular embodiments and not to limit the scope of the invention.

### Example 1

Isolation of the Nucleic Acid The original murine cDNA clone from which the full length sequence was derived was obtained from a mouse keratinocyte transit amplifying cell library (Genesis Inc., Aukland, New Zealand). Briefly, pelts obtained from 1-2 day postpartum neonatal BALB/cByJ mice were washed and incubated in trypsin (BRL Life Technologies, Gaithersburg, Md.) to separate the epidermis from the dermis. Epidermal tissue was disrupted to disperse cells, which were then resuspended in growth medium and centrifuged over Percoll density gradients prepared according to the manufacturer's protocol (Pharmacia, Uppsala, Sweden). Pelleted cells were labeled using Rhodamine 123 (Bertoncello et al., Exp Hematol. 13:999-1006, 1985), and analyzed by flow cytometry (Epics Elite Coulter Cytometry, Hialeah, Fla.). Single cell suspensions of rhodamine-labelled murine keratinocytes were then labeled with a cross-reactive anti-rat CD29 biotin monoclonal antibody (Pharmingen, San Diego, Calif.; clone Ha2/5). Cells were washed and incubated with anti-mouse CD45 Phycoerythrin-conjugated monoclonal antibody (Pharmingen; clone 30F11.1, 10 ug/ml) followed by labeling with streptavidin spectral red (Southern Biotechnology, Birmingham, Ala.). Sort gates were defined using listmode data to identify four populations: CD29 bright Rhodamine dull CD45 negative cells; CD29 bright Rhodamine bright CD45 negative cells; CD29 dull Rhodamine bright CD45 negative cells; and CD29 dull Rhodamine dull CD45 negative cells. Cells were sorted, pelleted and snap-frozen prior to storage at −80° C. This protocol was followed multiple times to obtain sufficient cell numbers of each population to prepare cDNA libraries. Skin stem cells and transit amplifying cells are known to express CD29, the integrin □1 chain. CD45, a leukocyte specific antigen, was used as a marker for cells to be excluded in the isolation of skin stem cells and transit amplifying cells. Keratinocyte stem cells expel the Rhodamine dye more efficiently than transit amplifying cells. The CD29 bright, Rhodamine dull, CD45 negative population (putative keratinocyte stem cells; referred to as KSCL) and the CD29 bright, Rhodamine bright, CD45 negative population (keratinocyte transit amplifying cells; referred to as TRAM) were sorted and mRNA was directly isolated from each cell population using the Quick Prep Micro mRNA purification kit (Pharmacia). A cDNA expression library was then prepared from the mRNA by reverse transcriptase synthesis using a Lambda ZAP cDNA library synthesis kit (Stratagene).

An independent clone was obtained from the murine thymic stromal cell Z210R.1 library. Confirmation of the position of the initiating methionine was obtained by inspection of a publicly available murine EST having the accession number AI1317448. Full length cloning yielded the polynucleotide sequence of SEQ ID NO:1 and corresponding amino acid sequence provided in SEQ ID NO:2.

The polynucleotide sequence of human DAKAR (referred to herein as huDAKAR, SEQ ID NO:7) was identified by PCR from human fetal liver cDNA (Clontech) using primers based on murine DAKAR sequence. The polynucleotide sequence for human DAKAR (SEQ ID NO:6) spans much of the putative open reading frame. Subsequent to the cloning of DAKAR, the sequence encoding huDAKAR was located in the GenBank database in a sequence for genomic DNA encoding human chromosome 21 (Nature, 405, May 18, 2000, pp 311-319). The precise location of huDAKAR is on chromosome 21 21q22.3, in clone contig KB657H6. This region of chromosome 21 shows synteny with mouse chromosome 16, where muDAKAR resides. Analysis of the sequence indicates that human and mouse DAKAR are 85% identical at the nucleotide level and 90% identical at the amino acid level. HuDAKAR is made up of 8 exons spanning a region of roughly 26,000 nucleotides. The first 6 exons encode the catalytic domain, exon 7 spans the unique region, and exon 8 (the largest exon) encompasses part of the unique region and all of the ankyrin repeats. Based on the published genomic sequence of human chromosome 21, the coding sequence of the final 25 amino acids of the amino terminus of huDAKAR was deduced yielding the full length clone having an amino acid sequence as provided in SEQ ID NO:7. Further analysis reveled a naturally occurring variant having a valine for methionine substitution at amino acid 666 of SEQ ID NO:7, a variant comprising amino acids 26 to 784 of SEQ ID NO:7, a variant comprising amino acids 1 to 750 of SEQ ID NO:7, and another variant comprising amino aicds 26 to 750 of SEQ ID NO:7.

Analysis of the polypeptide reveals a kinase domain from amino acids 22 to 302 of SEQ ID NO:7 based on comparison to known kinases. Amino acids 142 to 148 and amino acids 160 to 162 of SEQ ID NO:7 correspond to a conserved serine/threonine catalytic loop motif.

### Example 2

Use of DAKAR Polypeptides in an ELISA

Serial dilutions of DAKAR-containing samples (in 50 mM NaHCO₃, brought to pH 9 with NaOH) are coated onto Linbro/Titertek 96 well flat bottom E.I.A. microtitration plates (ICN Biomedicals Inc., Aurora, Ohio) at 100:1/well. After incubation at 4° C. for 16 hours, the wells are washed six times with 200:1 PBS containing 0.05% Tween-20 (PBS-Tween). The wells are then incubated with FLAG®-DAKAR binding partner at 1 μg/ml in PBS-Tween with 5% fetal calf serum (FCS) for 90 minutes (100:1 per well), followed by washing as above.

Next, each well is incubated with the anti-FLAG® ((monoclonal antibody M2 at 1 μg/ml in PBS-Tween containing 5% FCS for 90 minutes (100:1 per well), followed by washing as above. Subsequently, wells are incubated with a polyclonal goat anti-mIgG1-specific horseradish peroxidase-conjugated antibody (a 1:5000 dilution of the commercial stock in PBS-Tween containing 5% FCS) for 90 minutes (100:1 per well). The HRP-conjugated antibody is obtained from Southern Biotechnology Associates, Inc., Birmingham, Ala. Wells then are washed six times, as above.

For development of the ELISA, a substrate mix [100:1 per well of a 1:1 premix of the TMB Peroxidase Substrate and Peroxidase Solution B (Kirkegaard Perry Laboratories, Gaithersburg, Md.)] is added to the wells. After sufficient color reaction, the enzymatic reaction is terminated by addition of 2 N H₂SO₄ (50:1 per well). Color intensity (indicating DAKAR-binding activity) is determined by measuring extinction at 450 nm on a V Max plate reader (Molecular Devices, Sunnyvale, Calif.).

### Example 3

Polyclonal and Monoclonal Antibodies That Bind DAKAR

Rabbit anti-DAKAR polyclonal antibodies were prepared using peptides derived from the murine sequence of DAKAR (SEQ ID NO: 2). Five peptides were synthesized and isolated using conventional techniques well known in the art. The sequences and amino acid locations of the DAKAR peptides were amino acids 1 to 24, amino acids 52-79, amino acids 287 to 316, amino acids 497 to 515, and amino acids 769 to 784 of the polypeptide depicted in SEQ ID NO: 7.

One rabbit was immunized with 52-79, 287-316 and 769-784 and another was immunized with all five peptides. The peptide preparation, choice of adjuvant and immunization protocol are methods that are well known in the art and may be found, for example in Antibodies: A Laboratory Manual, Harlow and Land (eds.), Cold Spring Harbor Laboratory Press, Cold Spring Harbor, N.Y., (1988). The resultant polyclonal antisera is referred to as 3FEL for the animal immunized with the three DAKAR peptides and 5FEL for the antisera raised against all five peptides.

The following example illustrates a method for preparing monoclonal antibodies that bind DAKAR polypeptides. Other conventional techniques may be used, such as those described in U.S. Pat. No. 4,411,993. Suitable immunogens that may be employed in generating such antibodies include, but are not limited to, purified DAKAR polypeptide, an immunogenic fragment thereof, and cells expressing high levels of DAKAR polypeptide or an immunogenic fragment thereof. DNA encoding a DAKAR polypeptide can also be used as an immunogen, for example, as reviewed by Pardoll and Beckerleg in Immunity 3:165, 1995.

Rodents (BALB/c mice or Lewis rats, for example) are immunized with DAKAR polypeptide immunogen emulsified in an adjuvant (such as complete or incomplete Freund's adjuvant, alum, or another adjuvant, such as Ribi adjuvant R700 (Ribi, Hamilton, Mont.)), and injected in amounts ranging from 10-100 micrograms subcutaneously or intraperitoneally. DNA may be given intradermally (Raz et al., 1994, Proc. Natl. Acad. Sci. USA 91:9519) or intamuscularly (Wang et al., 1993, Proc. Natl. Acad. Sci. USA 90:4156); saline has been found to be a suitable diluent for DNA-based antigens. Ten days to three weeks days later, the immunized animals are boosted with additional immunogen and periodically boosted thereafter on a weekly, biweekly or every third week immunization schedule.

Serum samples are periodically taken by retro-orbital bleeding or tail-tip excision to test for DAKAR polypeptide antibodies by dot-blot assay, ELISA (enzyme-linked immunosorbent assay), immunoprecipitation, or other suitable assays, such as FACS analysis of inhibition of binding of DAKAR polypeptide to a DAKAR polypeptide binding partner. Following detection of an appropriate antibody titer, positive animals are provided one last intravenous injection of DAKAR polypeptide in saline. Three to four days later, the animals are sacrificed, and spleen cells are harvested and fused to a murine myeloma cell line, e.g., NS1 or preferably P3X63Ag8.653 (ATCC CRL-1580). These cell fusions generate hybridoma cells, which are plated in multiple microtiter plates in a HAT (hypoxanthine, aminopterin and thymidine) selective medium to inhibit proliferation of non-fused cells, myeloma hybrids, and spleen cell hybrids.

The hybridoma cells may be screened by ELISA for reactivity against purified DAKAR polypeptide by adaptations of the techniques disclosed in Engvall et al., (Immunochem. 8:871, 1971) and in U.S. Pat. No. 4,703,004. A preferred screening technique is the antibody capture technique described in Beckmann et al., (J. Immunol. 144:4212, 1990). Positive hybridoma cells can be injected intraperitoneally into syngeneic rodents to produce ascites containing high concentrations (for example, greater than 1 milligram per milliliter) of anti-DAKAR polypeptide monoclonal antibodies. Alternatively, hybridoma cells can be grown in vitro in flasks or roller bottles by various techniques. Monoclonal antibodies can be purified by ammonium sulfate precipitation, followed by gel exclusion chromatography. Alternatively, affinity chromatography based upon binding of antibody to protein A or protein G can also be used, as can affinity chromatography based upon binding to DAKAR polypeptide.

### Example 4

Expression of Endogenous DAKAR in Human Cells

Expression of endogenous human DAKAR in various cell types was examined using rabbit anti-murine DAKAR polyclonal antibodies. Results from those studies show that DAKAR is abundantly expressed in a variety of human cancer cell lines, including melanoma (WM164), colon carcinoma (HT29, Colo205) and ovarian carcinoma (IGROV-1). In comparison, DAKAR expression was low in the breast adenocarcinoma line MD231.

Further studies have shown no correlation in human cell lines that endogenously express DAKAR and those that have sensitivity or resistance to various TNF family cytokines. In a series of studies, treatment of 293 cells with staurosporine for 8 hr. resulted in the generation of a 50 kDa band that is strongly recognized by the anti-DAKAR antibody. A similar band is noted in WM164 cells, as well as in various dendritic cell (DC) populations. Preliminary studies indicated that message levels of DAKAR are upregulated in immature DCs and downregulated in cultured DCs. These results suggest the possibility that the additional lower molecular weight bands correspond to alternate splice forms of murine DAKAR.

### Example 5

Northern Blot Analysis

The tissue distribution of DAKAR mRNA can be investigated by Northern blot analysis, as follows. An aliquot of a radiolabeled probe (³²P-labeled PCR product derived from sequence contained within the DAKAR open reading frame) is added to both human and murine multiple tissue Northern blots (Clontech, Palo Alto, Calif.; Biochain, Palo Alto, Calif.). Hybridization is conducted as recommended by the manufacturer and using Clontech's ExpressHyb hybridization solution. The post hybridization wash protocol is also as described by the manufacturer.

Using the protocol described above, it was determined that a single transcript of approximately 4.0 kilobases (kb) was present in murine liver and kidney and to a lesser extent in lung and testis. A similar sized transcript was also detected in human kidney and pancreas and to a lesser extent in fetal liver, liver, lung and placenta.

### Example 6

Measuring Kinase Activity of DAKAR

Isolated DAKAR polypeptide or fusion proteins containing the isolated protein kinase domain of DAKAR can be used in an assay of protein kinase activity. Typically this would be carried out by combining DAKAR with radiolabeled ATP (γ³²p-ATP) and a magnesium salt in buffer solution containing a peptide or protein substrate. The peptide substrates are generally from 8-30 amino acids in length and may terminate at the N- or C-terminus with three or more lysine or arginine residues to facilitate binding of the peptide to phosphocellulose paper. The substrate may also be a protein known to be phosphorylated readily by DAKAR. Many such general kinase substrates are known, such as, α or β casein, histone H1, myelin basic protein, etc. After incubation of this reaction mixture at 20-37° C. for a suitable time, the transfer of radioactive phosphate from ATP to the substrate protein or substrate peptide may be monitored, by spotting of the reaction mixture onto phosphocellulose paper, and subsequent washing of the paper with a dilute solution of phosphoric acid, in the case of a peptide substrate, or by application of the reaction products to a gel electrophoresis system followed by autoradiographic detection in the case of proteins. Other methods are available to conveniently measure the DAKAR-mediated transfer of phosphate to substrate proteins, such as the scintillation proximity assay, these methods are well known to those practiced in the art.

### Example 7

cDNA Construct Comprising DAKAR-FLAG® Fusion Protein and Catalytic Domain Mutant of DAKAR

A cDNA construct comprising residues 1 to 786 of SEQ ID NO.2 (murine DAKAR) fused at the 3′ end to FLAG® (SEQ ID NO:3) was created (DAKAR-FLAG®) and inserted into the expression vector pDC412 using conventional techniques well known in the art. The DAKAR-FLAG®/pDC412 construct was expressed in 293 EBNA cells under standard culture conditions.

A dominant negative mutant DAKAR cDNA clone (referred to herein as DAKAR-A51) was prepared in order to determine whether the kinase catalytic domain played an active role in inducing apoptosis. Using standard site-directed mutagenesis techniques, a mutagenized form of DAKAR was prepared wherein the codon for the invariant lysine (amino acid 51 of SEQ ID NO:2) of the ATP-binding pocket was exchanged for an alanine. Mutation of the ATP-binding pocket obviates ATP binding and consequently prevents subsequent phosphorylation of downstream substrates.

### Example 8

Overexpression of DAKAR is Associated with Apoptosis.

Overexpression of the DAKAR-FLAG® fusion protein and the dominant negative mutant DAKAR-A51 (as described in Example 7) in 293 in EBNA cells activates pro-apoptotic pathways, such as the cleavage of caspase-3, cleavage of the intracellular caspase substrates fodrin and poly(ADP-ribose)polymerase (PARP). Expression of an irrelevant cytosolic protein of similar size in the same vector (GID myc) under similar conditions was without effect.

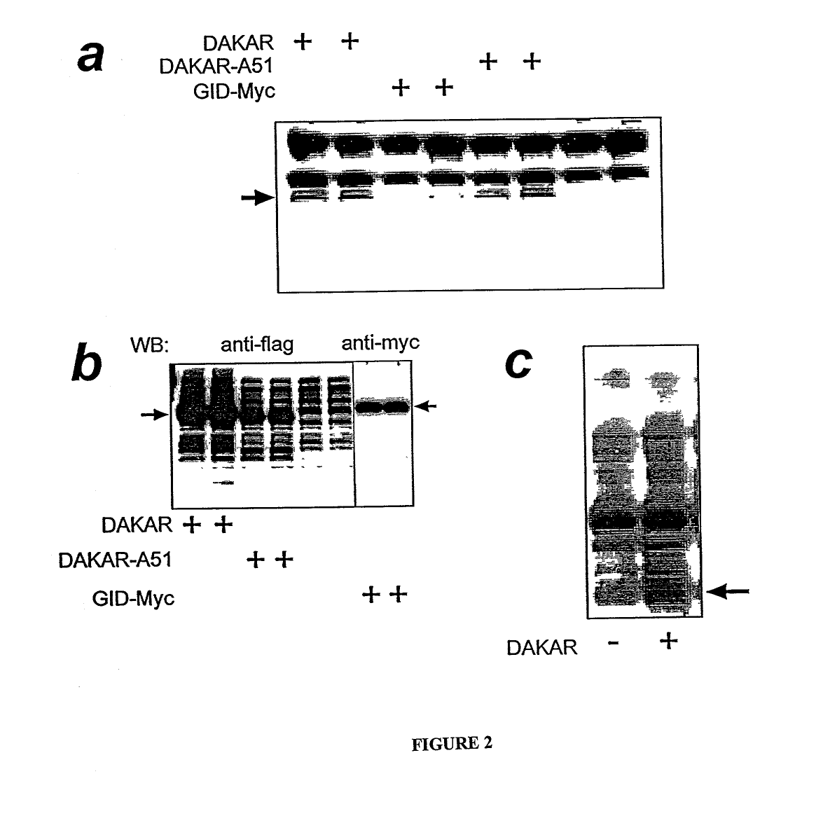

As illustrated in FIG. 2a, duplicate cultures of 293/EBNA were transfected with expression plasmids encoding DAKAR with an N-terminal FLAG® extension (lanes 1, 2), a DAKAR-FLAG® in which the invariant lysine 51 of the ATP binding site lysine at position 51 of SEQ ID NO:7 was mutated to alanine (lanes 5, 6), or an irrelevant control protein GID fused to a c-myc epitope tag (lanes 3, 4). Control cells (lanes 7, 8) were transfected with the vector plasmid alone. After 48 hrs, the cells were lysed in a NP-40-containing buffer and aliquots of the lysates were electrophoresed on SDS-polyacrilamide gels. The separated proteins were electrophoretically transferred to nitrocellulose membranes for analysis by Western immunoblotting with an antibody against the apoptotic target protein fodrin (Chemicon International Inc., Temecula, Calif.). The position of the 120 kDA fodrin cleavage product is indicated by the arrow at left. FIG. 2b shows further aliquots of the samples described above which were immunoblotted with anti-FLAG® antibodies (Eastman Kodak Co., Scientific Imaging Systems Division, New Haven, Conn.—lanes 1-6) to visualize recombinant DAKAR proteins (the major translation products of about 98 kDa are indicated with an arrow), or with anti-c-myc (lanes 7, 8) to visualize GID-myc (arrowed on right). FIG. 2c further shows aliquots of lysates from cells transfected with DAKAR (lane 2) or control vector (lane 1) that were electrophoresed and immunoblotted with anti-caspase-3 antibody (Pharmingen, San Diego, Calif.) to detect the 17 kDa cleavage product produced during apoptosis. Notably, expression of DAKAR was as effective an inducer of PARP (anti-PARP antibodies available from Pharmingen) or fodrin cleavage, as well as other well-characterized apoptotic stimuli, such as TNFα, TRAIL or anti-Fas antibody. (Anti-TNFα monoclonal antibody available from R&D Systems, Minneapolis, Minn. and anti-Fas monoclonal antibodies available from Upstate Biotech)

Although expressed at lower levels than wild-type DAKAR, mutated DAKAR also induced apoptotic changes similar to those described above, thereby demonstrating that DAKAR does not require a functional kinase domain for inducing apoptosis. The finding that the kinase activity of DAKAR is not required for apoptotic activity is consistent with similar findings reported for RIP, RIP2/RICK and RIP3.

### Example 9

Truncated Mutants of DAKAR Lose the Capacity to Induce Apoptosis

A number of truncated DAKAR mutants were prepared to determine whether the ability to induce apoptosis is localized to a certain region of the molecule. Truncation mutants were prepared using standard techniques. Truncation mutants of DAKAR encoding the amino terminus-proximal 1-295 amino acids (the entire kinase domain), or the carboxy terminus-proximal 525 amino acids (the entire ankyrin-repeat domain), beginning at Methionine residue 262, (DAKAR 1-295 and DAKAR 262-786) were constructed in the pDC412 expression vector. Each construct (DAKAR 1-295 and DAKAR 262-786), contained the FLAG® sequence immediately prior to the termination codon.

The expression vectors along with full-length FLAG®-DAKAR and the catalytically inactive FLAG®-DAKAR-A51 were transfected into 293/EBNA cells. Control cells were transfected with pDC412 vector containing no insert. 24h prior to lysis, some cells were pre-treated with TRAIL. Apoptosis was assessed by western-immunoblot detection of fodrin cleavage product. Relative expression of the recombinant proteins was assessed by FLAG® immunoblotting.

The results of this experiment demonstrated that both the N-terminal kinase and C-terminal ankyrin repeat domains of DAKAR, but not DAKAR kinase activity are required to induce apoptosis and to potentiate TRAIL-induced apoptosis. These results indicate that, although the kinase functionality is not required per se, other structural elements in both the kinase and non-kinase portions of the molecule are required for its apoptotic function.

### Example 10

DAKAR Potentiates the Activity of Pro-Apoptotic Stimuli

Cultures of 293/EBNA cells were transfected with DAKAR expression plasmids as described in Example 8. Prior to lysis, selected cultures were stimulated with agents known to induce apoptosis, e.g., anti-Fas antibody CH11 (500 ng/ml for 24 h), TNFα (100 ng/ml for 18 h), UV irradiation (80,000 J/cm² for 6 hrs, followed by 24 h in ambient light), or TRAIL (300 ng/ml for 24 h). Cell lysates were prepared for immunoblotting as described in Example 7. Apoptosis was assessed by visualization of the 85 kDa cleavage product of the apoptotic target poly-ADP ribose polymerase (PARP).

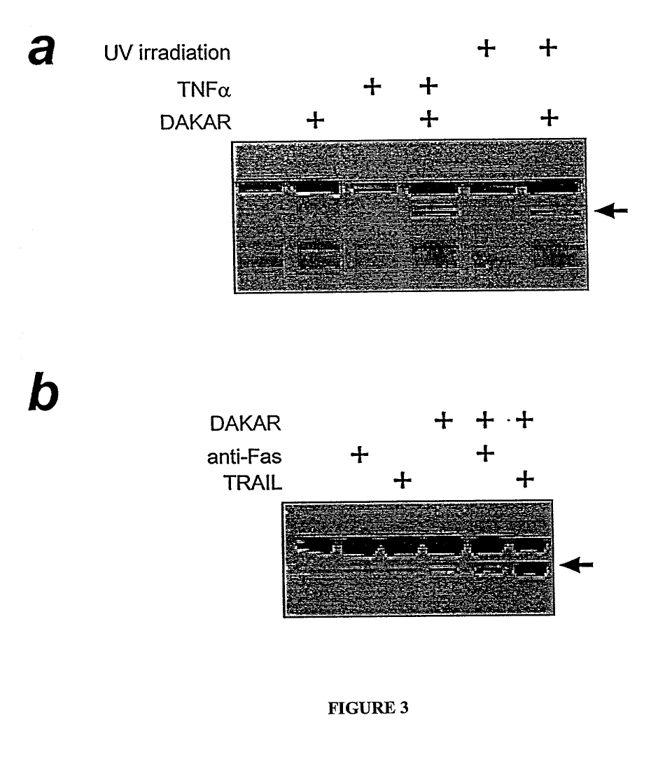

The results demonstrate that increased expression of DAKAR by transient transfection can more than double the amount of PARP in a cell when expressed in conjunction with secondary apoptotic stimuli, namely, TRAIL, TNFα, engagement of Fas/Apo-1 or ultraviolet irradiation, when compared to DAKAR treatment alone or treatment of the cells with the apoptotic stimuli alone (FIG. 3).

### Example 11

Overexpression of DAKAR Activates NF-κB

This example demonstrates that overexpression of murine DAKAR activates NF-κB in vitro. 293 EBNA cells were co-transfected with a NF-κB-luciferase reporter vector and either a control vector or decreasing amounts of DAKAR (SEQ ID NO:2), dominant negative DAKAR-A51 (essential, conserved lysine 51 has been mutated to alanine) or DAKAR 1-295. The overall amount of plasmid DNA was held constant at 1 ug per transfection by addition of the appropriate control vector. The transfections were performed in triplicate. 24 hours after transfection, cell extracts were prepared and luciferase assays were performed according to the manufacturer's instructions (Promega, Madison, Wis.). Protein content was also evaluated by conventional methods using a commercially available kit (Pierce Chemical Co., Rockford Ill.). The relative luciferase activity was normalized to protein content and triplicate values were averaged.

Full length wild-type DAKAR gave an inversely related dose-dependent response of NF-κB-luciferase, possibly due to cell mortality attributable to the apoptotic effects of overexpressed DAKAR, whereas the kinase dead DAKAR mutant DAKAR-A51 did not activate NF-κB-luciferase at a detectable level. The kinase domain mutant of DAKAR, DAKAR 1-295, showed decreased activation of NF-κB-luciferase relative to full length, but was still able to activate NF-κB-luciferase. These data suggest the requirement for an intact catalytic region of DAKAR for NF-κB activation, which is distinct from RIP, RIP2 and RIP3, which do not require kinase activity.

### Example 12

DAKAR Knockout Mice and Gross Phenotypic Morphology

Mice lacking DAKAR were generated by gene targeting in embryonic stem cells using standard techniques. Methods and techniques for the generation of knockout mice are well known in the art (see, for example, Ramirez-Soltis, R., et al., Nature 378:720-724, 1995 and Hogan, B., et al., Manipulating the Mouse Embryo: A Laboratory Manual, Cold Spring Harbor Laboratory, 1994; and Lexicon Incorporated MOUSE KIT™ Instruction Manual). Briefly, a DAKAR gene targeting construct was prepared using conventional techniques, such as those described in Hasty, P., et al., “Gene targeting vectors for mammalian cells” in Gene Targeting: a Practical Approach, A. L. Joyner, ed. IRL Press: Oxford, pp 1-31, (1993). Specifically, DAKAR genomic clones were obtained from a murine 129Svj Lambda Genomic Library (Stratagene Ltd., Cambridge, UK) using murine cDNA from a keratinocyte transit amplifying (TRAM) library from a BalbC mouse as a probe. Restriction maps were obtained of 2 overlapping genomic clones spanning a region of 20 Kb, which contained the entire coding sequence in 8 exons. All the intron/exon junctions were sequenced. The DAKAR homologous recombination vector was constructed from a 5.4 Kb HindIII-Xba1 and a 1.3 Kb Nhe1-EcoR1 fragment from the genomic clones so as to delete exons 2-7 and part of exon 8 thereby eliminating amino acids 62-492 (counting ATG as amino acid one). The vector also contained PGKNeo and TK cassettes as selection markers. The DAKAR HRV was electroporated into a 129ES cell line @200V and the cells were exposed to 175 ug/ml Geneticin and 2uM Gancyclovir. After 10 days, colonies were picked, pooled and screened by PCR using the Neo oligo #17576 and the genomic oligo #39695:

#17576: 5′ GAA TGG GCT GAC CGC TTC CTC G 3′ (Neo cassette)

#39695: 5′ CAT CTC CAA GCC CCA TGT TAC TG 3′ (genomic)

Positive DAKAR +/−ES colonies were subsequently confirmed by genomic Southern using a BamH1 digest of the ES cell DNA and a 3 Kb EcoR1-Stu1 genomic fragment as a probe (the KO allele is 6 Kb larger than the wt allele). Five DAKAR +/−ES cell lines were injected into C57B1/6 blasts, which were carried to term by Swiss Webster females. Chimeric males were bred to C57B1/6 females to assay for transmission of the KO allele. One line went germline (#26.9).

Mice carrying the KO allele were identified by PCR of ear-punch DNA using the following oligo sets:

Wt allele:

#37103: 5′GTC CTC ACG CCT CAA GCG CG 3′ (exon 7)

#37105: 5′CAG TGA CAG CGA TCC TCT GGA G 3′ (exon 7)

KO allele:

#17576 (see above)

#32952: 5′ TTC TCC TCT ATG AGC AGC TTG AC 3′ (exon 8)

129XB6 hybrid DAKAR +/−mice were bred to produce 129XB6 DAKAR −/− mice. Timed pregnancies were used to obtain DAKAR −/− at different embryonic stages for study. The KO allele was backcrossed onto the C57B1/6 background 4 times. DAKAR−/−ES cells were injected into C57B1/6 blastocysts to assess the contribution of DAKAR−/− cells to varying cell lines in the resulting chimera. A Mouse/Hamster Radiation Hybrid Panel (Research Genetics, Inc., Huntsville, Ala.) was screened using muDAKAR-specific PCR oligos to identify the chromosomal location of the DAKAR gene. DAKAR mapped to an area between the markers D16Mit106 and D16Mit95 on the distal end of mu chromosome 16.

Mice lacking DAKAR display a specific defect in keratinocyte differentiation, proliferation and/or other related pathway that affects all squamous epithelia and results in perinatal lethality. Genotyping identified a homozygous mutant, suggesting that DAKAR^{-/-} pups were dying just prior to or after birth. Grossly, all e17.5 and e18.5 DAKAR mutants have thickened non-wrinkled skin, poorly defined oral and auricular orifices, and the tail is fused to the skin in the area of the rectum and urethra opening resulting in no visible rectal or urethra orifice (anal and urethra atresia). Atresia is defined as the congenital absence or closure of a normal body orifice or tubular organ. Additionally, there was a high degree of atresia surrounding the mouth and ears of the mutant embryos. Although a ridge line was evident at the site of the mouth, this ridge was impenetrable. In contrast, wild-type embryos at this stage have open mouths with well-developed snouts and whiskers. Moreover, the limbs appeared rigid and somewhat stunted and in some embryos the toes appear to be fused. Finally, there is a linear red friable streak along the dorsal midline of e18.5 and newborn mutants.

Microscopically, e17.5 and e18.5 DAKAR^{-/-} mutants consistently have markedly thickened corneal epithelium of the surface epidermis compared to wild-type embryos. This hyperplasia of the suprabasal layer may explain the tautness and lack of skin folds seen in the embryos. Moreover, the terminally differentiated outermost skin layer, the stratum corneum, which gives the ridged appearance of the skin surface, appears to be absent in the mutant skin. The epidermis at dorsal midline in e18.5 and newborn mutants is thin, with degenerating cells in most layers of the epidermis. In contrast, the epidermis not at midline in the e18.5 and newborn mutants is thickened. The corneal epithelium between the digits of all four feet is partially fused. In addition, the corneal epithelium of the oral cavity and lips (oral atresia), esophagus (esophageal atresia), anterior portion of the stomach (atresia of the anterior stomach) are fused.

Dissection of mutant embryos has indicated that all major organs are present, including lungs, liver, heart, kidneys, thymus, spleen, stomach, colon, and testes. Additionally, the urinary bladders are extremely full and distended, which is consistent with the urethra and anal atresia observed as a result of increased proliferation and/or lack of differentiation of cells around these and other orifices.

### Example 13

DAKAR is Cleaved by Caspase 3 and Caspase 8 in vitro.

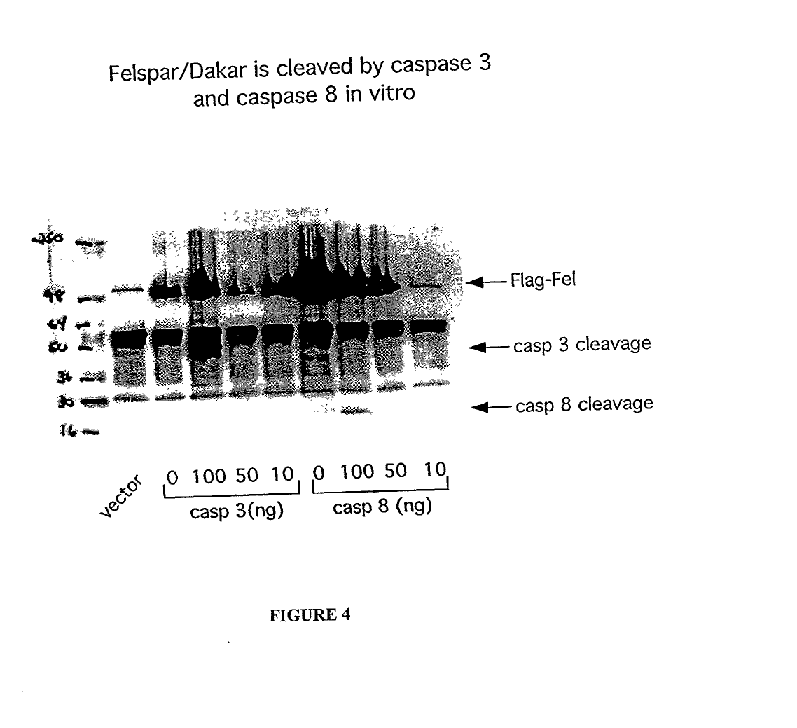

Wild-type FLAG®-tagged DAKAR or empty vector was transfected into 293 EBNA cells using DEAE dextran. Forty-eight hours after transfection, cells were washed once in cold PBS and lysed in buffer containing 50 mM Hepes, pH 7.4, 5 mM MnCl₂, 10 mM MgCl₂, 5 mM EGTA, 2 mM EDTA, 100 mM NaCl, 5 mM KCl, 1.0% NP-40, 1 mM PMSF, 1 ug/ml leupeptin, 20 mM β-glycerophosphate, 20 mM NaF, 0.3 mM Na₃PO₄, 1% aprotinin, 1 mM DTT and 10% glycerol. After measurement of total protein using conventional methods (e.g., Bradford, Pierce, Roockford, Ill.), FLAG®-tagged DAKAR was immunoprecipitated using FLAG®-M2 coated beads at 4C (FIG. 4).

Immune complexes were washed three times in lysis buffer, followed by two washes in caspase assay buffer containing 50 mM Nacl, 40 mM β-glycerophosphate, 10 mM Hepes, pH 7.4, 5 mM EGTA, 2 mM MgCl₂ and 10 mM DTT. Complexes were resuspended in 10 μl caspase assay buffer and incubated with 100 ng, 50 ng or 10 ng purified recombinant active caspase 3 or caspase 8 (Pharmingen, San Diego, Calif.) or buffer alone for one hour at 37° C. Reactions were stopped by addition of Laemmli sample buffer and analyzed by SDS-PAGE and Western blotting with rabbit anti-DAKAR polyclonal antibody. As shown in FIG. 4, in vitro incubation of DAKAR with 100 ng caspase 3 resulted in formation of approximately 40 and 45 kDa cleavage products, as indicated by the arrow. Furthermore, incubation of DAKAR with 100 ng caspase 8 resulted in the formation of a 22 kDa cleavage product, as indicated by the arrow. Together these results demonstrate that DAKAR is cleaved by caspase 3 and caspase 8.

### Example 14

Immunohistochemical Analysis of DAKAR^{-/-} Mutants

This example is a summary of the immunohistochemical analysis of various subcellular markers, including keratinocyte differentiation markers. Stratified or cornified epithelium, such as the stratified squamous epithelium of the skin, possesses a unique morphology. Only the innermost basal layer of the epidermis has the capacity for DNA synthesis and mitosis and through an undefined pathway, a basal cell is triggered to undergo terminal differentiation and migrate from the basal layer to the outermost stratum corneum layer where a series of morphological and biochemical changes culminate in the production of dead, flattened, enucleated squames, which are sloughed from the surface and are continually replaced by inner cells differentiating outwardly. Cornified epithelial cells build an extensive cytoskeletal structure, which is characterized by the presence of keratin filaments. In the epidermis, there are two major pairs of keratins; one of which is expressed in dividing cells and the other expressed in terminally differentiated cells. Basal epidermal cells display a keratin network composed of the type II keratin K5 and type I keratin K14, and as basal epidermal cells differentiate, they downregulate expression of K5/K14 and induce expression of a new set of differentiation-specific keratins, such as type II keratin K1 and type I keratin K10. As epidermal cells proceed through differentiation, they switch on the expression of additional type II keratin, K2 and type I keratin (56 kDa).

The following antibodies were used to mark proteins associated with differentiation on formalin fixed paraffin embedded sections of e18.5 wild-type and mutant skin: Fillagrin, Ki-67, Keratin 10, Keratin 14, 1 kB alpha, 1 kB beta and Re1A in nucleus. Results of the immunocytochemistry are presented in Table 2.

TABLE 2

| | Stratum | | Stratum | | Stratum | | Stratum | |
|---|---|---|---|---|---|---|---|---|
| | Basal | | Spinosum | | granulosum | | corneum | |
| | WT | KO | WT | KO | WT | KO | WT | KO |
| Involucrin | − | − | − | + | + | + | + | NA |
| Fillagrin | − | − | + | + | + | + | + | NA |
| Ki-67 | + | + | − | ? | − | − | − | NA |
| Keratin 10 | − | − | + | Weak | + | − | − | NA |
| Keratin 14 | + | + | − | − | − | + | − | NA |
| IkB alpha | + | + | + | + | + | + | − | NA |
| IkB beta | − | + | + | + | + | + | − | NA |
| RelA in | + | − | − | − | − | − | − | NA |
| nucleus | | | | | | | | |

### EXAMPLE 15

RT-PCR Analysis of DAKAR Expression in Human Tissue cDNA Panels

This example describes RT-PCR amplification from tissue-specific cDNA libraries to detect DAKAR cDNA sequences. RT-PCR was performed using GeneAmp 5700 as per the manufacturer's instructions on human tissue cDNA panels (Clontech). DAKAR transcripts are expressed in a wide variety of human fetal and adult cells, including human brain, kidney, liver, lung, pancreas, placenta, skeletal muscle, colon, PBLs, prostate, small intestine, testis, thymus, tonsil, as well as fetal lung, liver, kidney, heart and thymus. By far the greatest expression level was seen in prostate tissue.

### Example 16

Antisense Inhibition of DAKAR Nucleic Acid Expression

In accordance with the present invention, a series of oligonucleotides are designed to target different regions of the DAKAR mRNA molecule, using the nucleotide sequence of SEQ ID NO:2 and 7 as the basis for the design of the oligonucleotides. The oligonucleotides are selected to be approximately 10, 12, 15, 18, or more preferably 20 nucleotide residues in length, and to have a predicted hybridization temperature that is at least 37 degrees C. Preferably, the oligonucleotides are selected so that some will hybridize toward the 5′ region of the mRNA molecule, others will hybridize to the coding region, and still others will hybridize to the 3′ region of the mRNA molecule. Methods such as those of Gray and Clark (U.S. Pat. Nos. 5,856,103 and 6,183,966) can be used to select oligonucleotides that form the most stable hybrid structures with target sequences, as such oligonucleotides are desirable for use as antisense inhibitors.

The oligonucleotides may be oligodeoxynucleotides, with phosphorothioate backbones (internucleoside linkages) throughout, or may have a variety of different types of internucleoside linkages. Generally, methods for the preparation, purification, and use of a variety of chemically modified oligonucleotides are described in U.S. Pat. No. 5,948,680. As specific examples, the following types of nucleoside phosphoramidites may be used in oligonucleotide synthesis: deoxy and 2′-alkoxy amidites; 2′-fluoro amidites such as 2′-fluorodeoxyadenosine amidites, 2′-fluorodeoxyguanosine, 2′-fluorouridine, and 2′-fluorodeoxycytidine; 2′-O-(2-methoxyethyl)-modified amidites such as 2,2′-anhydro[1-(beta-D-arabino-furanosyl)-5-methyluridine], 2′-O-methoxyethyl-5-methyluridine, 2′-O-methoxyethyl-5′-O-dimethoxytrityl-5-methyluridine, 3′-O-acetyl-2′-O-methoxy-ethyl-5′-O-dimethoxytrityl-5-methyluridine, 3′-O-acetyl-2′-O-methoxyethyl-5′-O-dimethoxytrityl-5-methyl-4-triazoleuridine, 2′-O-methoxyethyl-5′-O-dimethoxytrityl-5-methylcytidine, N4-benzoyl-2′-O-methoxyethyl-5′-O-dimethoxytrityl-5-methylcytidine, and N4-benzoyl-2′-O-methoxyethyl-5′-O-di-methoxytrityl-5-methylcytidine-3′-amidite; 2′-O-(aminooxyethyl) nucleoside amidites and 2′-O-(dimethylaminooxyethyl) nucleoside amidites such as 2′-(dimethylaminooxyethoxy) nucleoside amidites, 5′-O-tert-butyldiphenylsilyl-O²-2′-anhydro-5-methyluridine, 5′-O-tert-butyl-diphenylsilyl-2′-O-(2-hydroxyethyl)-5-methyluridine, 2′-O-([2-phthalimidoxy)ethyl]-5′-t-butyl-diphenyl-silyl-5-methyluridine, 5′-O-tert-butyldiphenylsilyl-2′-O-[(2-formadoximinooxy)ethyl]-5-methyluridine, 5′-O-tert-butyldiphenylsilyl-2′-O-[N,N-dimethyl-aminooxyethyl]-5-methyluridine, 2′-O-(dimethylaminooxy-ethyl)-5-methyluridine, 5′-O-DMT-2′-O-(dimethylaminooxyethyl)-5-methyluridine, and 5′-O-DMT-2′-O-(2-N,N-dimethylaminooxyethyl)-5-methyluridine-3′-[(2-cyanoethyl)-N,N-diisopropyl-phosphor-amidite]; and 2′-(aminooxyethoxy) nucleoside amidites such as N2-isobutyryl-6-O-diphenyl-carbamoyl-2′-O-(2-ethylacetyl)-5′-O-(4,4′-dimethoxy-trityl)guanosine-3′-[(2-cyanoethyl)-N,N-diiso-propylphosphoramidite].

Modified oligonucleosides may also be used in oligonucleotide synthesis, for example methylenemethylimino-linked oligonucleosides, also called MMI-linked oligonucleosides; methylene-dimethylhydrazo-linked oligonucleosides, also called MDH-linked oligonucleosides; methylene-carbonylamino-linked oligonucleosides, also called amide-3-linked oligonucleosides; and methylene-aminocarbonyl-linked oligonucleosides, also called amide-4-linked oligonucleosides, as well as mixed backbone compounds having, for instance, alternating MMI and P=O or P=S linkages, which are prepared as described in U.S. Pat. Nos. 5,378,825, 5,386,023, 5,489,677, 5,602,240 and 5,610,289. Formacetal- and thioformacetal-linked oligonucleosides may also be used and are prepared as described in U.S. Pat. Nos. 5,264,562 and 5,264,564; and ethylene oxide linked oligonucleosides may also be used and are prepared as described in U.S. Pat. No. 5,223,618. Peptide nucleic acids (PNAs) may be used as in the same manner as the oligonucleotides described above, and are prepared in accordance with any of the various procedures referred to in Peptide Nucleic Acids (PNA): Synthesis, Properties and Potential Applications, Bioorganic & Medicinal Chemistry, 1996, 4:5-23; and U.S. Pat. Nos. 5,539,082, 5,700,922, and 5,719,262.

Chimeric oligonucleotides, oligonucleosides, or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”. Some examples of different types of chimeric oligonucleotides are: [2′-O-Me]-[2′-deoxy]-[2′-O-Me] chimeric phosphorothioate oligonucleotides, [2′-O-(2-methoxyethyl)]-[2′-deoxy]-[2′-O-(methoxyethyl)] chimeric phosphorothioate oligonucleotides, and [2′-O-(2-methoxy-ethyl)phosphodiester]-[2′-deoxy-phosphoro-thioate]-[2′-O-(2-methoxyethyl)phosphodiester] chimeric oligonucleotides, all of which may be prepared according to U.S. Pat. No. 5,948,680. In one preferred embodiment, chimeric oligonucleotides (“gapmers”) 18 nucleotides in length are utilized, composed of a central “gap” region consisting of ten 2′-deoxynucleotides, which is flanked on both sides (5′ and 3′ directions) by four-nucleotide “wings”. The wings are composed of 2′-methoxyethyl (2′-MOE) nucleotides. The internucleoside (backbone) linkages are phosphorothioate (P=S) throughout the oligonucleotide. Cytidine residues in the 2′-MOE wings are 5-methyl-cytidines. Other chimeric oligonucleotides, chimeric oligonucleosides, and mixed chimeric oligonucleo-tides/oligonucleosides are synthesized according to U.S. Pat. No. 5,623,065.

Oligonucleotides are preferably synthesized via solid phase P(III) phosphor-amidite chemistry on an automated synthesizer capable of assembling 96 sequences simultaneously in a standard 96 well format. The concentration of oligonucleotide in each well is assessed by dilution of samples and UV absorption spectroscopy. The full-length integrity of the individual products is evaluated by capillary electrophoresis, and base and backbone composition is confirmed by mass analysis of the compounds utilizing electrospray-mass spectroscopy.

The effect of antisense compounds on target nucleic acid expression can be tested in any of a variety of cell types provided that the target nucleic acid is present at measurable levels. This can be routinely determined using, for example, PCR or Northern blot analysis. Cells are routinely maintained for up to 10 passages as recommended by the supplier. When cells reached 80% to 90% confluency, they are treated with oligonucleotide. For cells grown in 96-well plates, wells are washed once with 200 microliters OPTI-MEM-1 reduced-serum medium (Gibco BRL) and then treated with 130 microliters of OPTI-MEM-1 containing 3.75 g/mL LIPOFECTIN (Gibco BRL) and the desired oligonucleotide at a final concentration of 150 nM. After 4 hours of treatment, the medium is replaced with fresh medium. Cells are harvested 16 hours after oligonucleotide treatment. Preferably, the effect of several different oligonucleotides should be tested simultaneously, where the oligonucleotides hybridize to different portions of the target nucleic acid molecules, in order to identify the oligonucleotides producing the greatest degree of inhibition of expression of the target nucleic acid.

Antisense modulation of DAKAR nucleic acid expression can be assayed in a variety of ways known in the art. For example, DAKAR mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+mRNA. Methods of RNA isolation and Northern blot analysis are taught in, for example, Ausubel, F. M. et al., Current Protocols in Molecular Biology, Volume 1, pp. 4.1.1-4.2.9 and 4.5.1-4.5.3, John Wiley & Sons, Inc., 1996. Real-time quantitative (PCR) can be conveniently accomplished using the commercially available ABI PRISM 7700 Sequence Detection System, available from PE-Applied Biosystems (Foster City, Calif.) and used according to manufacturer's instructions. This fluorescence detection system allows high-throughput quantitation of PCR products. As opposed to standard PCR, in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., JOE or FAM, obtained from either Operon Technologies Inc., Alameda, Calif. or PE-Applied Biosystems, Foster City, Calif.) is attached to the 5′ end of the probe and a quencher dye (e.g., TAMRA, obtained from either Operon Technologies Inc., Alameda, Calif. or PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular (six-second) intervals by laser optics built into the ABI PRISM 7700 Sequence Detection System. In each assay, a series of parallel reactions containing serial dilutions of mRNA from untreated control samples generates a standard curve that is used to quantitate the percent inhibition after antisense oligonucleotide treatment of test samples. Other methods of quantitative PCR analysis are also known in the art. DAKAR protein levels can be quantitated in a variety of ways well known in the art, such as immunoprecipitation, Western blot analysis (immunoblotting), ELISA, or fluorescence-activated cell sorting (FACS). Antibodies directed to DAKAR polypeptides can be prepared via conventional antibody generation methods such as those described herein. Immunoprecipitation methods, Western blot (immunoblot) analysis, and enzyme-linked immunosorbent assays (ELISA) are standard in the art (see, for example, Ausubel, F. M. et al., Current Protocols in Molecular Biology, Volume 2, pp. 10.16.1-10.16.11, 10.8.1-10.8.21, and 11.2.1-11.2.22, John Wiley & Sons, Inc., 1991).

### Example 17

Treatment of Alopecia Using Agonists of DAKAR

In light of DAKAR's expression in epithelial tissues and the overt phenotype of DAKAR knock out mice, skin grafts were performed by transferring a patch of skin from either DAKAR −/− knock out mice or wild type littermates onto a wild type background mouse. Interestingly, while the skin grafts were capable of growth, the DAKAR −/− grafts failed to maintain or grow hair, and had a similar appearance as alopecia indicating that the loss of DAKAR expression interferes with hair follicle development and/or maintenance.

Thus, in light of the failure of hair to grow in the absence of DAKAR expression, agonists of DAKAR can be used to induce hair growth in patients in need thereof, for example, to treat alopecia, including but not limited to alopecia areata, male pattern baldness, and/or alopecia capitis totalis. The reverse is also possible, more specifically, antagonists of DAKAR can be used to prevent unwanted growth of hair.

### Example 18

Diagnosis and/or Treatment of Immune Diseases or Disorders Associated with DAKAR

Variable expression of a gene product can oftentimes be associated with a disease condition. DAKAR −/− cells were tested for upregulated or downregulated expression of secondary nucleic acids, namely mRNAs. We utilized an Affymetrix Gene Chip oligonucleotide array, which provides an analysis of mRNA abundance of roughly 12,000 mouse genes. mRNA was prepared from the skins of 2 wild-type and 2 DAKAR−/− E18.5 embryos. Skin from each wild-type embryo was independently compared to skin from each mutant embryo, generating four separate analyses.

In total, 94 identified genes were expressed in embryonic DAKAR −/− skin but not wild-type, and 27 genes were found to be expressed in wild-type but not DAKAR −/− animals. Table 3 presents data of representative genes that were either upregulated or downregulated in duplicate wild-type or duplicate mutant samples. Each number is a ratio of expression of the gene in DAKAR −/− cell line divided by a wild type control. When the ratio was less than one, the fractional number was divided into one and changed to a negative number to represent the higher expression in wild type cells versus mutant.

TABLE 3
Changes in gene expression in skin from DAKAR −/− tissues

| | | 269^{-/-} | 272^{-/-} | 269^{-/-} | 272^{-/-} |
|---|---|---|---|---|---|
| Genbank | | vs. | vs. | vs. | vs. |
| Acc # | Name | 271^{+/+} | 271^{+/+} | 270^{+/+} | 270^{+/+} |
| M36120 | Keratin 19 | 10.3 | 9.6 | 9.8 | 9.7 |
| AB012042 | Keratin 6 beta | 35.2 | 25.6 | 21.1 | 15.6 |
| AB012033 | Keratin 6 alpha | 67 | 54.9 | 13.7 | 10.8 |
| K02108 | Keratin 6 alpha | 59 | 38.7 | 17 | 14.2 |
| AF053235 | Keratin 16 | 22 | 15.9 | 22.9 | 16.5 |
| AF021836 | Hair keratin Hb5 | −5.4 | −4.2 | −4.6 | −4.8 |
| AB033744 | Novel type II | −6.1 | −5.4 | −5.4 | −4.8 |
| | keratin | | | | |
| X91825 | Sprr protein 1B | 7.9 | 6.5 | 6.3 | 5.1 |
| AJ005559 | Sprr protein 2A | 18.1 | 16.5 | 10.4 | 8.6 |
| AJ005561 | Sprr protein 2C | 5 | 5 | 8.1 | 5.9 |
| AJ005562 | Sprr protein 2D | 12.9 | 10.6 | 6.7 | 4.9 |
| AJ005566 | Sprr protein 2H | 3.1 | 3.1 | 3.2 | 3.2 |
| X99251 | Repetin | 15.9 | 10.4 | 10.2 | 6.6 |
| M83218 | S100 protein A8 | 12.4 | 10.6 | 16.7 | 13.2 |
| M83219 | S100 protein A9 | 16.8 | 15.1 | 20.5 | 16 |
| AV007820 | S100 protein A13 | 8.4 | 9.1 | 3.9 | 9.3 |
| AF004941 | S100 protein A3 | −3.5 | −3.1 | −3.8 | −3.2 |
| Y18723 | Prss18, ser protease | 15.3 | 8.3 | 6.7 | 3.7 |
| X03505 | Saa3 | 13.6 | 6.1 | 9.5 | 3.4 |
| X93035 | Chitinase 3 like p. | 7.5 | 6.6 | 5.9 | 5.1 |
| AI854500 | Pro-platelet basic p. | 18.3 | 5.9 | 13.2 | 3.5 |
| AA273423 | Pro-platelet basic p. | 27.9 | 9.2 | 17.5 | 5.1 |
| M28698 | Keratin 19 | 12.1 | 7.6 | 10.2 | 5.9 |

As noted in the above examples, targeted disruption of DAKAR in mice results in perinatal lethality associated with abnormalities in skin and other stratified epithelial tissues. It is particularly noteworthy that in skin from E18.5 embryos, a severe hyperplasia and parakeratosis is observed, reminiscent of an inflammatory response. In addition, there is a thickening of the spinous and granular skin layers and an absence of the outermost cornified layers in DAKAR−/− skin, suggesting a block in keratinocyte differentiation. Moreover, the pattern of gene expression set forth in Table 3 of the DAKAR knock out mouse model is consistent with gene expression profiles in damaged, inflamed and/or psoriatic skin. Thus, the foregoing gene expression pattern can be used to diagnose and/or provide prognosis for patients afflicted with a skin disease or disorder associated with aberrant expression of DAKAR. Furthermore, agonists of DAKAR can be used to treat skin diseases associated with aberrant DAKAR expression and having such a gene expression profile.

### Example 19

Treatment of Immune Diseases or Disorders Not Associated With DAKAR

In order to evaluate immune cells from DAKAR −/− mice hematopoietic cells were isolated from DAKAR −/− embryonic livers. It was shown that there was an increase in B220+ and CD43+cells relative to control animals, in addition, a significant subset of these cells were also CD24+(heat stable antigen (HAS)) and ectopeptidase+ (BP-1). The expression of markers B220, CD43 with CD24 are indicative of B lineage commitment by precursor cell (Hunte et al., 1998, Eur. J. Immunol., 28:3850-3856). The four markers together correlate with a B cell population that is at the pre-B cell stage (Lu et al., 1998, Eur. J Immunol., 28:1755-1761). These results suggest that in the absence of DAKAR, a signaling molecule involved in apoptotic cell death, there is an increase in the B cell lineage commitment by precursor cells. Thus, antagonists of DAKAR can be used as B cell growth stimulants to increase the number of B cells in a patient in need thereof. Specific examples of diseases or disorders where this is desired, include but are not limited to infectious diseases, cancer therapy, coadministration with a vaccine, or immune deficiencies among others. Conversely, where it is desired to decrease the number of B cells in a patient in need thereof, an agonist of DAKAR can be administered. Examples where such a treatment is desired include, but is not limited to autoimmunity.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### Example 20

Treatment of Squamous Cell Carcinoma

Skin grafts were performed by transferring a patch of skin from either DAKAR −/− knock out mice or wild type littermates onto a wild type background mouse (see Example 17). Ten weeks post engraftment, in addition to failing to support normal hair growth, the hair follicles took on a dysplastic appearance, the sebaceous glands showed characteristics of hyperplasia, and one graft developed squamous cell carcinoma.

Squamous cell carcinoma is typically a malignant tumor of keratinocytes. Most squamous cell carcinomas arise from sun-induced precancerous lesions known as actinic keratoses, but can also be induced by immunosuppression and/or chronic inflammation, among other causes. Other types of squamous cell carcinomas include Bowen's disease which is an in situ squamous cell carcinoma, Keratoacanthoma, and metastatic squamous cell carcinomas.

The presence of dysplasia, hyperplasia, and squamous cell carcinoma in the DAKAR knockout grafts indicates that DAKAR is a regulator of epithelial cell growth, development and proliferation. Accordingly, in one embodiment of the invention, increasing expression of DAKAR or administration of an agonist of DAKAR can be used to treat squamous cell carcinoma. In another embodiment, it is contemplated that an agonist of DAKAR can be administered to a patient who exhibits typical pre-squamous cell carcinoma conditions such as, for example, sunburn or chronic inflammation, as a prophylactic treatment for epithelial cell disorders. Likewise, it is contemplated that DAKAR antagonists can be administered to patients having epithelial cell irregularities that are indicative a predisposition of, or precancerous stage prior to the onset of cancer.

Equivalents and References

The present invention is not to be limited in scope by the specific embodiments described herein, which are intended as single illustrations of individual aspects of the invention, and functionally equivalent methods and components are within the scope of the invention. Those skilled in the art will recognize, or be able to ascertain using no more than routine experimentation, many equivalents to the specific embodiments of the invention described herein. Indeed, various modifications of the invention in addition to those described herein will become apparent to those skilled in the art from the foregoing description. Such equivalents are intended to be within the scope of the following claims.

All publications, patents and patent applications mentioned in this specification are herein incorporated by reference into the specification to the same extent as if each individual publication, patent or patent application was specifically and individually indicated to be incorporated herein by reference.

What is claimed is:

1. An isolated polypeptide selected from the group consisting of:
(a) an isolated polypeptide comprising an amino acid sequence having at least 95% identity with the amino acid sequence of SEQ ID NO:7;
(b) an isolated polypeptide comprising the amino acid sequence of SEQ ID NO:7;
(c) an isolated polypeptide having the amino acid sequence of SEQ ID NO:7; and
(d) an isolated polypeptide comprising an amino acid sequence havint at least 95% identity with amino acids 22 to 302 of the sequence depicted in SEQ ID NO:7.

2. An isolated nucleic acid selected from the group consisting of:
(a) an isolated nucleic acid comprising a nucleotide sequence encoding a DAKAR polypeptide, wherein said polypeptide has at least 90% identity with the amino acid sequence of SEQ ID NO:7;
(b) an isolated nucleic acid comprising a nucleotide sequence having at least 90% identity to a nucleotide sequence encoding the polypeptide of SEQ ID NO:7;
(c) an isolated nucleic acid comprising a nucleotide sequence having at least 90% identity with nucleotides 1 to 906 of SEQ ID NO:6;
(d) an isolated nucleic acid comprising a nucleotide sequence encoding the polypeptide of SEQ ID NO:7; and
(e) an isolated nucleic acid comprising the nucleotide sequence of SEQ ID NO:6.

3. The nucleic acid of claim 2 further comprising vector nucleic acid sequences.

4. The nucleic acid of claim 2 further comprising nucleic acid sequences encoding a heterologous polypeptide.

5. An isolated antibody immunospecific to a polypeptide consisting of the amino acid sequence of SEQ ID NO:7.

6. The antibody of claim 5 wherein the antibody is a monoclonal antibody.

7. A recombinant vector comprising a polynucleotide sequence according to claim 2.

8. A host cell transfected with the recombinant vector of claim 7.

9. A host cell genetically engineered to express a polypeptide according to claim 1, wherein a recombinant heterologous control element is operably linked to a polynucleotide encoding said polypeptide.

10. A method of producing a polypeptide comprising the steps of culturing the host cell of claims 8 or 9 under conditions promoting expression of said polypeptide, and isolating said polypeptide.

11. A method for identifying candidate compounds that modulate the biological activity of a polypeptide according to claim 1 comprising
(a) mixing a test compound with said polypeptide; and
(b) determining whether the test compound modulates the biological activity of said polypeptide.

12. A method comprising administering to a patient in need thereof a compound comprising an agonist of a polypeptide according to claim 1.

13. The method of claim 12, wherein the patient has psoriasis, melanoma, or squamous cell carcinoma.

14. A kit comprising a compound which selectively binds to a polypeptide of claim 1 and instructions for use.

15. A method for detecting the presence of a nucleic acid molecule of claim 2 in a sample, comprising the steps of:
(a) contacting the sample with a nucleic acid probe or primer which selectively hybridizes to the nucleic acid molecule; and
(b) determining whether the nucleic acid probe or primer binds to a nucleic acid molecule in the sample.

16. The method of claim 15, wherein the sample comprises mRNA molecules and is contacted with a nucleic acid probe.
